(19) United States
(12) Patent Application Publication
(10) Pub. No.: US 2012/0129179 A1
(43) Pub. Date: May 24, 2012

(54) SCN5A SPLICING FACTORS AND SPLICE VARIANTS FOR USE IN DIAGNOSTIC AND PROGNOSTIC METHODS

(75) Inventor: Samuel Dudley, Chicago, IL (US)

(73) Assignee: THE BOARD OF TRUSTEES OF THE UNIVERSITY OF ILLINOIS, Urbana, IL (US)

(21) Appl. No.: 13/291,826

(22) Filed: Nov. 8, 2011

Related U.S. Application Data
(63) Continuation-in-part of application No. PCT/US2010/034271, filed on May 10, 2010.
(60) Provisional application No. 61/176,665, filed on May 8, 2009.
(60) Provisional application No. 61/253,916, filed on Oct. 22, 2009.
(60) Provisional application No. 61/415,040, filed on Nov. 18, 2010.

(51) Int. Cl. C12Q 1/68; G01N 33/566

(52) U.S. Cl. 435/6.12; 436/501; 435/7.1

(57) ABSTRACT

Provided herein are methods of identifying a subject at risk for arrhythmias, heart failure or sudden cardiac death. In exemplary aspects, the method comprises the step of determining a level of splicing factor hLuc7a, splicing factor RBM25 and/or PERK in a biological sample obtained from the subject, wherein an increased level, compared to a control level, indicates a risk for arrhythmia or heart failure. The invention also provides methods of diagnosing hypertrophic cardiomyopathy (HCM), or a risk therefor, in a subject. The method comprises the step of determining a level of E28D, RBM25, PRPF40A, or LUC7L3, or a combination thereof, in a biological sample obtained from the subject, wherein an increased level is indicative of the subject having HCM or a risk therefor. Diagnostic kits comprising binding agents for the markers are furthermore provided.

### CROSS-REFERENCE TO RELATED APPLICATIONS

This application is a continuation-in-part of International Patent Application No. PCT/US2010/034271, filed May 10, 2010, which claims the benefit of U.S. Provisional Application No. 61/176,665, filed May 8, 2009, and U.S. Provisional Application No. 61/253,916, filed Oct. 22, 2009. This application also claims priority to U.S. Provisional Application No. 61/415,040, filed Nov. 18, 2010. The disclosures of each of these applications are incorporated herein by reference in their entirety.

### STATEMENT OF U.S. GOVERNMENTAL INTEREST

This invention was made with U.S. government support under National Institutes of Health Grant Nos. RO1 HL085558 and RO1 HL085520. The government has certain rights in this invention.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 123 kilobytes ACII (Text) file named “45336A_SeqListing.txt,” created on Nov. 8, 2011.

### BACKGROUND

Heart disease is the number one cause of death in the United States, surpassing even cancer. The National Center for Chronic Disease Prevention and Health Promotion estimates that approximately 950,000 Americans die of cardiovascular disease every year, accounting for more than 40 percent of all deaths. One form of cardiovascular disease, arrhythmia, is associated with very high levels of morbidity and mortality. Sudden arrhythmic death claims more than 300,000 lives each year.

Arrhythmia is defined as abnormal beating of the heart. Heart beat, a complex process of contraction and expansion, is controlled by electrical impulses, which are, in turn, regulated by the flow of specific ions (K⁺, Na⁺ and Ca²⁺) across cellular membranes. Integral membrane proteins, or channels, act as gates, controlling the flow of ions in and out of cells. Sodium, calcium and potassium channels play pivotal roles in generating cardiac action potential, which triggers contraction. Ion channel dysfunction resulting from genetic mutation is a primary cause of arrhythmia.

Voltage-gated sodium channels are pore-forming membrane proteins responsible for the initiation and propagation of action potentials in excitable membranes in nerve, skeletal muscle and heart cells. The controlled gating of sodium channels in response to membrane depolarization is necessary for normal electrical signaling and establishing of intercellular communication. The cardiac voltage-sensitive sodium (Na⁺) channel is composed of α and β subunits. The gene encoding the α-subunit, SCN5A, has been cloned and found to consist of 28 exons spanning over 80 kb of DNA. The α-subunit (or its isoforms) contains four homologous repeated domains (D1-D4), each with six transmembrane segments (S1-S6). The α-subunit protein alone forms a functional channel when expressed in mammalian expression systems. The four repeated domains are hypothesized to assemble as a pseudotetrameric structure with the permeation pathway situated at the center. The protein is responsible for the rapid influx of sodium ions that initiate and propagate action potential in the heart and the large peak sodium influxes responsible for excitability and conduction in myocardium and special conduction tissues.

The human voltage-gated cardiac Sodium channel α-subunit, referred to as Nav1.5, which is encoded by the gene SCN5A, is by far the most abundant Sodium channel protein in the human heart. The SCN5A gene has been cloned and characterized in 1992 by Gellens et al. (Proceedings of the National Academy of Sciences of the United States of America 89:554-558 (1992)). SCN5A consists of 28 exons spanning approximately 80 kb found by Wang et al. (Genomics 34:9-16 (1996)), which described the sequences of all intron/exon boundaries and a dinucleotide repeat polymorphism in intron 16. George et al. (Cytogenet. Cell Genet. 68:67-70 (1995)) mapped the SCN5A gene to 3p21 by fluorescence in situ hybridization, thus making it an important candidate gene for long QT syndrome-3 in 1995. Nav1.5 is responsible for the rapid influx of sodium ions that initiates and propagates action potentials in heart, large peak inward sodium current that underlies excitability and conduction in working myocardium and special conduction tissue. Interventions that modulate sodium current have potent physiologic effects. Mutations in the human SCN5A gene cause the long QT syndrome (LQT) and idiopathic ventricular fibrillation (IVF). Mutations in SCN5A that generate truncated, misprocessed, or dysfunctional proteins produce the Brugada variant of idiopathic ventricular fibrillation. Schott et al. (Nat. Genet. 23:20-21 (1999)) reported a mutation in the SCN5A gene that segregated with progressive cardiac conduction defect (PCCD) in an autosomal dominant manner in a large French family.

Alternative splicing, the process by which multiple messenger RNA (mRNA) isoforms are generated from a single pre-mRNA species is an important means of regulating gene expression. Alternative splicing plays a central role in numerous biological processes such as sexual differentiation in Drosophila and apoptosis in mammals [Lopez (1998) Ann. Rev. Genet. 32:279-305]. Aberrant splicing generates abnormal mRNAs which are either unstable or code for defective or deleterious protein isoforms which are frequently implicated in the development of human disease [Lopez (1998) Ann. Rev. Genet. 32:279-305; Charlet (2002) Mol. Cell. 10:45-53].

Shang et al. (Circ. Res., 101:1146-1154, 2007) reported the detection of three SCN5A mRNA variants in human heart in addition to the full length form, and two of them were found to be increased in heart failure patients. SCN5A mRNA variants resulted from splicing at cryptic splice sequences in the terminal exon of SCN5A (i.e., exon 28). In comparison with the full-length Na⁺ channel, all three new variants were shorter and encoded prematurely truncated Na⁺ channel proteins missing the segments from domain IV, S3 or S4 to the C-terminus. The presence of the variants caused the reduced abundance of the full-length SCN5A mRNA.

### SUMMARY OF THE INVENTION

The present disclosure is based in part on the discovery that splicing factors hLuc7a and RBM 25 play a role in the abnormal splicing of the voltage-gated cardiac sodium channel α-subunit (SCN5A) gene. The presence of abnormal SCN5A splice variants down-regulates expression of the SCN5A gene. Thus, in one embodiment, provided herein is a method of inhibiting downregulation of the SCN5A gene in a cell comprising contacting the cell with a compound that inhibits activity of splicing factor hLuc7a, splicing factor RBM25 and/or protein kinase R-like ER kinase (PERK) in an amount effective to inhibit downregulation of the SCN5A gene. In some embodiments, the method is an in vivo method.

In one embodiment, the method inhibits or reverses an effect of hypoxia or angiotensin II in the cell. In another embodiment, downregulation of splicing factor hLuc7A or splicing factor RBM25 downregulates expression of abnormal SCN5A splice variants. In some embodiments, the abnormal SCNA splice variant is selected from the group consisting of E28B, E28C and E28D. In another embodiment, downregulation of PERK upregulates expression of full-length SCN5A.

It has been shown that RBM25 is a splicing factor that binds tightly to the canonical RNA sequence CGGGC(A) (Zhou et al., Mol. Cell. Biol., 28:5924-5936, 2008). Therefore, in some embodiments, the compound that inhibits activity of splicing factor hLuc7a, splicing factor RBM25 and/or PERK reduces splicing factor interaction with the canonical sequence CGGGCA in a genomic polynucleotide encoding SCN5A.

In some embodiments, the cell is a blood cell, a muscle cell or a neuron. In some embodiments, the cell is a leukocyte, a macrophage or a cardiac cell.

In another embodiment, described herein is a method of identifying a compound that inhibits transcription of an abnormal SCN5A splice variant in a cell comprising the step of contacting the cell that comprises a SCN5A gene with a test compound in the presence and absence of a splicing factor selected from the group consisting of hLuc7a and RBM25, wherein the splicing factor binds to the SCN5A gene in the absence of the test compound, and determining the presence or absence of an abnormal splice variant in the cell, wherein the absence of the abnormal splice variant in the cell identifies the test compound as a compound that inhibits transcription of an abnormal splice variant.

In some embodiments, the cell is a blood cell, a muscle cell or a neuron. In some embodiments, the cell is a leukocyte, a macrophage or a cardiac cell.

Any compound that inhibits the activity of hLuc7a, RBM25 and/or PERK is contemplated for use in the methods described herein. Exemplary compounds include, but are not limited to, inhibitory oligonucleotides, antibodies and small molecules.

Both prophylactic and therapeutic methods of treating a subject at risk of (or susceptible to) a disorder or having a disorder associated with aberrant or unwanted target gene expression or activity (e.g., in exemplary embodiments, expression of abnormal SCN5A splice variants) are provided. For example, in one embodiment, a prophylactic method of treating arrhythmia in a subject is provided, the method comprising identifying the subject as being at risk for developing arrhythmia and administering to a subject at risk of arrhythmia a compound that inhibits the activity of hLuc7a, RBM25 and/or PERK in an amount effective to prevent arrhythmia. In another aspect, the method alternatively comprises the step of identifying an individual at risk of arrhythmia. In such embodiments, the identifying step comprises screening for the presence of an abnormal SCN5A splice variant in a biological sample of the subject, wherein the presence of the abnormal splice variant identifies the subject as being at risk for developing arrhythmia. For example, the presence of one or more SCNA splice variants E28B (SEQ ID NO: 7), E28C (SEQ ID NO: 8) and/or E28D (SEQ ID NO: 9) in the biological sample identifies the subject as being at risk for developing arrhythmia. Thus the screening step, in some embodiments, comprises obtaining a biological sample from the subject and analyzing nucleic acid from the sample for the presence of an abnormal splice variant.

In some embodiments, the identifying step comprises determining a level of hLuc7a, RBM25 and/or PERK in a biological sample of a subject, wherein an increase in the level of hLuc7a, RBM25 and/or PERK in the sample identifies the subject as being at risk for developing arrhythmia.

Methods of treating arrhythmia in a subject are also provided. For example, in one embodiment, the method comprises administering an effective amount of a compound that that inhibits activity of splicing factor hLuc7a, splicing factor RBM25 and/or PERK to the subject.

In some embodiments, the subject is human. Practice of the methods described herein in other mammalian subjects, especially mammals that are conventionally used as models for demonstrating therapeutic efficacy in humans (e.g., primate, porcine, canine, or rabbit animals), is also contemplated. In some embodiments, the subject is suffering from a cardiac disorder, including but not limited to, heart failure, ischemia, myocardial infarction, congestive heart failure, arrhythmia, transplant rejection and the like. In one embodiment, the subject is suffering from heart failure. In another embodiment, the subject is suffering from arrhythmia.

In some embodiments, a method to monitor the efficacy of treatment is provided. In such embodiments, the method comprises determining a level of hLuc7a, RBM25 and/or PERK in a biological sample of a subject before and after treatment with a compound that inhibits activity of hLuc7a, RBM25 and/or PERK. In such a method, a change in the level of hLuc7a, RBM25 and/or PERK in the sample taken after treatment compared to the level of hLuc7a, RBM25 and/or PERK before treatment indicates efficacy of the treatment. In some embodiments, a first biological sample is obtained from the subject to be treated prior to initiation of therapy or part way through a therapy regime. Alternatively, in some embodiments, a first biological sample is obtained from a subject known not to suffer from a condition being treated. In some embodiments, the second biological sample is obtained in a similar manner, but at a time following onset of therapy. The second biological sample, in some embodiments, is obtained at the completion of, or part way through therapy, provided that at least a portion of therapy takes place between the isolation of the first and second biological samples. A decrease in the level of hLuc7a, RBM25 and/or PERK in the second biological sample (e.g., post-treatment) compared to the level of hLuc7a, RBM25 and/or PERK in the first biological sample (e.g., prior to treatment or from a subject known not to suffer from the condition being treated) indicates a degree of effective therapy.

The level of abnormal SCN5A splice variants in a biological sample, in some embodiments, is analyzed in a similar manner to monitor efficacy of treatment, with a decrease in the level of abnormal SCN5A splice variants in the sample indicates effective therapy. In such embodiments, the method to monitor efficacy of treatment comprises determining a level of abnormal SCN5A splice variants in a biological sample of a subject before and after treatment with a compound that inhibits activity of hLuc7a, RBM25 and/or PERK. In such a method, a change in the level of abnormal SCN5A splice variants in the sample taken after treatment compared to the level of abnormal SCN5A splice variant in the sample before treatment indicates efficacy of treatment. A decrease in the level of abnormal SCN5A splice variants in the second biological sample (e.g., post-treatment) compared to the level of abnormal SCN5A splice variants in the first biological sample (e.g., prior to treatment or from an individual known not to suffer from the condition being treated) indicates a degree of effective therapy.

Alternatively, the level of full-length SCN5A in a biological sample post-treatment is analyzed to monitor efficacy of treatment, with an increase in the level of full-length SCN5A in the sample indicates effective therapy. In such embodiments, the method to monitor efficacy of treatment comprises determining a level of full-length SCN5A in a biological sample of a subject before and after treatment with a compound that inhibits activity of hLuc7a, RBM25 and/or PERK. In such a method, a change in the level of full-length SCN5A in the sample taken after treatment compared to the level of full-length SCN5A in the sample before treatment indicates efficacy of treatment. An increase in the level of full-length SCN5A in the second biological sample (e.g., post-treatment) compared to the level of full-length SCN5A in the first biological sample (e.g., prior to treatment or from an individual known not to suffer from the condition being treated) indicates a degree of effective therapy.

In yet another alternative embodiment, a level of hypoxia and/or angiotensin II in a biological sample post-treatment is analyzed to monitor efficacy of treatment, with a decrease in the level of hypoxia and/or angiotensin II in the sample indicates effective therapy. In such embodiments, the method to monitor efficacy of treatment comprises determining a level of hypoxia or angiotensin II in a biological sample of a subject before and after treatment with a compound that inhibits activity of hLuc7a, RBM25 and/or PERK. In such a method, a change in the level of hypoxia or angiotensin II in the sample taken after treatment compared to the level of hypoxia or angiotensin II in the sample before treatment indicates efficacy of treatment. A decrease in the level of hypoxia or angiotensin II in the second biological sample (e.g., post-treatment) compared to the level of hypoxia or angiotensin II in the first biological sample (e.g., prior to treatment or from an individual known not to suffer from the condition being treated) indicates a degree of effective therapy.

The use of a compound that inhibits activity of hLuc7a, RBM25 and/or PERK in the manufacture of a medicament for the treatment of arrhythmia is also provided. Also provided is the use of a compound that inhibits activity of hLuc7a, RBM25 and/or PERK for the treatment of arrhythmia. In some embodiments, the compound is administered to a subject at risk for developing arrhythmia.

In addition to the foregoing embodiments, the invention furthermore provides diagnostic and prognostic methods relating to SCN5A splicing factors and splice variants, among others. The invention provides methods of identifying a subject at risk for arrhythmias or heart failure. In exemplary embodiments, the method comprises the step of determining a level of splicing factor hLuc7a, splicing factor RBM25 and/or PERK in a biological sample from the subject, wherein an increased level of hLuc7a, RBM25 and/or PERK in the sample compared to a control sample (e.g., a comparable sample from an individual known not to be at risk for arrhythmia or heart failure) indicates a risk for arrhythmia or heart failure.

For purposes herein, the level may be an expression level of splicing factor hLuc7a, splicing factor RBM25 and/or PERK, or may be an activity level of splicing factor hLuc7a, splicing factor RBM25 and/or PERK, e.g., a binding activity or an enzymatic activity.

In additional or alternative embodiments, the method of identifying a subject at risk for arrhythmias or heart failure comprises the step of determining a level of a chaperone protein in a biological sample from the subject, wherein an increased level of the chaperone protein in the sample compared to a control sample indicates an increased risk for arrhythmia or heart failure. In exemplary embodiments, the chaperone protein is CHOP or calnexin. For purposes herein, the level may be an expression level of the chaperone protein or an activity level of the chaperone protein, e.g., a binding activity or an enzymatic activity.

In additional or alternative embodiments, the method of identifying a subject at risk for arrhythmias or heart failure comprises the step of determining a level of a full length transcript of SCN5A gene or of a splice variant of the SCN5A gene. Splice variants of the SCN5A gene are further described herein and in the art. See, e.g., U.S. Application Publication No. 2007/0212723 A1. In exemplary aspects, the method comprises determining a level of a full length transcript of SCN5A gene, and a decreased level of the full length transcript of the SCN5A gene indicates an increased risk for arrhythmia or heart failure. In exemplary aspects, the method comprises determining a level of a splice variant of the SCN5A gene, and an increased level of the splice variant indicates an increased risk for arrhythmia or heart failure. In specific aspects, the splice variant of the SCN5A gene is a splice variant produced from alternative splicing within Exon 28 of the SCN5A gene. In specific aspects, the splice variant is a SCN5A Exon 28 B splice variant (a.k.a., E28B), a SCN5A Exon 28 C splice variant (a.k.a., E28C), or a SCN5A Exon 28 D splice variant (a.k.a., E28D). Such splice variants of the SCN5A gene are further described herein.

For purposes herein, the level may be an expression level of a full length transcript of SCN5A gene or of a splice variant of the SCN5A gene. Suitable methods of determining expression levels of transcripts of a gene are described further herein and in the art, and include direct methods of determining levels of transcripts (e.g., quantitative PCR) and indirect methods of determining levels of transcripts (e.g., Western blotting for translated protein or peptide products of the transcripts). The level may be an activity level of a full-length transcript of the SCN5A gene that is determined via measurement of, e.g., sodium current.

In exemplary aspects, the method comprises screening for the presence of an abnormal SCN5A splice variant in a biological sample of the subject, wherein the presence of the abnormal splice variant identifies the subject as being at risk for developing arrhythmia. For example, the presence of one or more SCNA splice variants E28B (SEQ ID NO: 7), E28C (SEQ ID NO: 8) and/or E28D (SEQ ID NO: 9) in the biological sample identifies the subject as being at risk for developing arrhythmia. Thus the screening step, in some embodiments, comprises obtaining a biological sample from the subject and analyzing nucleic acid from the sample for the presence of an abnormal splice variant.

Arrhythmias and heart failure are related to sudden cardiac death (SCD). For example, SCD is responsible for about 50% of deaths from heart failure and often is the first expression of coronary disease. See, Sovari et al., “Sudden Cardiac Death,” e-medicine Cardiology, article 151907, updated Nov. 4, 2010; and Zheng et al., Circulation 104: 2158-2163 (2001). A common cause of SCD is ventricular arrhythmia, including, for example, ventricular tachycardia (VT), in which the resting heart rate is faster than normal, ventricular fibrillation (VF), in which there is uncoordinated contraction of the cardiac muscle of the ventricles in the heart, making the muscles quiver rather than contract properly, or an arrhythmic condition in which both VT and VF are present. See, Wedro, B., “Sudden Cardiac Arrest (Sudden Cardiac Death),” medicine.net, Kulick and Soppler, eds.

Accordingly, the invention also provides methods of identifying a subject at risk for sudden cardiac death. In exemplary embodiments, the method comprises the step of determining a level of splicing factor hLuc7a, splicing factor RBM25 and/or PERK in a biological sample from the subject, wherein an increased level of hLuc7a, RBM25 and/or PERK in the sample compared to a control sample (e.g., a comparable sample from an individual known not to be at risk for SCD) indicates a risk for SCD. Further aspects of this invention are provided herein.

The invention moreover provides a method of diagnosing a subject with hypertrophic cardiomyopathy (HCM) or determining a subject's risk of developing HCM. In exemplary embodiments, the method comprises the step of determining a level of E28D, RBM25, PRPF40A, or LUC7L3, or a combination thereof, in a sample obtained from the subject, wherein an increased level is indicative of the subject having HCM or an increased risk of developing HCM.

The invention also provides a method of prognosticating a subject with HCM. In exemplary embodiments, the method of prognosticating the HCM subject is a method of determining the subject's risk for sudden cardiac death, arrhythmias (e.g., atrial arrhythmias), heart failure, and/or dilated cardiomyopathy. In exemplary embodiments, the method comprises the step of determining a level of E28D, RBM25, PRPF40A, or LUC7L3, or a combination thereof, in a sample obtained from the subject, wherein a modified level, as compared to a control sample, is indicative of a poor prognosis, indicative of need for treatment, and/or indicative of an increased risk for sudden cardiac death, arrhythmias (e.g., atrial arrhythmias), heart failure, and/or dilated cardiomyopathy.

### BRIEF DESCRIPTION OF THE FIGURES

FIG. 1 is a schematic representation of the splice variants identified in the 5′ end of the human SCN5A gene. The map shows the genomic structure of SCN5A with untranslated (open bars) or translated (closed bars) transcribed sequences and nontranscribed sequences (lines). Splicing patterns for each of the three exon 1 isoforms are identified.

FIG. 2 provides cDNA sequences for E1A, E1B1, E1B2, E1B3, E1B4, E2A, E2B1 and E2B2.

FIG. 3 is a schematic representation of the splice variants identified in the 3′ end of the human SCN5A gene. Above the map shows the genomic structure of SCN5A with untranslated (open bars) or translated (closed bars) transcribed sequences and nontranscribed sequences (lines). Splicing patterns for each of the four exon 28 isoforms are identified.

FIG. 4 provides cDNA sequences for E28A (FIGS. 4A and 4B), E28B (FIG. 4C), E28C (FIG. 4D), and E28D (FIG. 4E).

### DETAILED DESCRIPTION

The present disclosure is based in part on the discovery that splicing factors hLuc7a and RBM25 play a role in the abnormal splicing of the SCN5A gene. Some of the examples provided herein demonstrate that upregulation of mRNA splicing factors hLuc7A and RBM25 mediate abnormal SCN5A splicing, and that the increased expression of abnormal SCN5A splice variants activates the unfolded protein response via protein kinase R-like endoplasmic reticulum kinase (PERK), leading to degradation of the full-length SCN5A mRNA through the unfolded protein response (UPR) pathway. Thus, splicing factors hLuc7a and RBM25 and PERK are attractive targets for the treatment and/or prevention of arrhythmia in heart failure patients.

SCN5A Splicing Factors and Transducer of the UPR

Data provided herein evidence that splicing factors hLuc7A and RBM25, as well as the transducer of the unfolded protein response (UPR), PERK, are associated with abnormal splicing of the SCN5A gene. Luc7A (NCBI Gene ID No. 51747) is also known as LUC7L3; LUC7-like 3; CRA; CROP; LUCIA; hLuc7A; CREAP-1; and OA48-18. Exemplary mRNA sequences of hLuc7A are set forth herein as SEQ ID NOs: 34 and 35 but may also found in the NCBI's nucleotide database as Accession No. NM_{—}006107.3 and as Accession No. NM_{—}016424.4. Exemplary amino acid sequences of hLuc7A are set forth herein as SEQ ID NOs: 36 and 37 but may also be found in the NCBI's Protein database as Accession No. NP_{—}006098.2 and as Accession No. NP_{—}057508.2. RBM25 (NCBI Gene ID No. 58517) is also known RNA binding motif protein 25; 5164; NET52; RNPC7; Snu71; RED120; fSAP94; MGC105088; and MGC117168. An exemplary mRNA sequence of RBM25 is set forth herein as SEQ ID NO: 38 but may be found in the NCBI's nucleotide database as Accession No. NM_{—}021239.2. An exemplary amino acid sequence of RBM25 is set forth herein as SEQ ID NO: 39 but may be found in the NCBI's Protein database as Accession No. NP_{—}067062.1. PERK (NCBI Gene ID No. 9451) is also known as eukaryotic translation initiation factor 2-alpha kinase 3, EIF2AK3, protein kinase R-like endoplasmic reticulum kinase, PKR-like ER kinase; PEK; WRS; and DKFZp781H1925. An exemplary mRNA sequence of PERK is set forth herein as SEQ ID NO: 40 but may also be found on the NCBI's nucleotide database as Accession No. NM_{—}004836.5. An exemplary amino acid sequence of PERK is set forth herein as SEQ ID NO: 41 but may also be found on the NCBI's Protein database as Accession No. NP_{—}004827.4.

SCN5A Splice Variants

SCN5A splice variants in or near the 5′ and 3′ untranslated region (UTR) of the mRNA sequence of SCN5A are associated with heart diseases, such as arrhythmia and heart failure. SCN5A splice variants include E1B1 (SEQ ID NO. 1), E1B2 (SEQ ID NO. 2), E1B3 (SEQ ID NO. 3), E1B4 (SEQ ID NO. 4), E2B1 (SEQ ID NO. 5), E2B2 (SEQ ID NO. 6), E28B (SEQ ID NO. 7), E28C (SEQ ID NO. 8), or E28D (SEQ ID NO. 9).

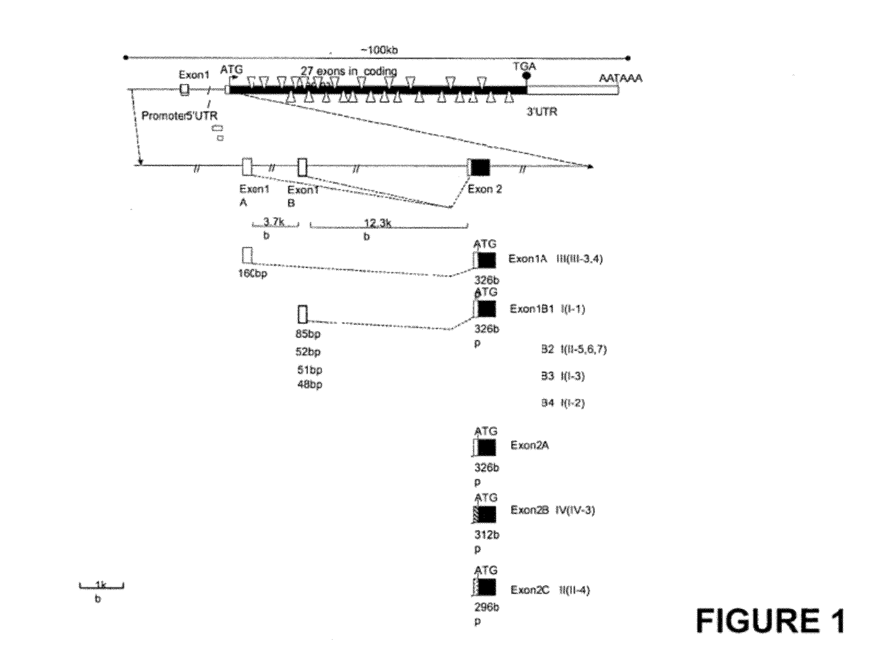

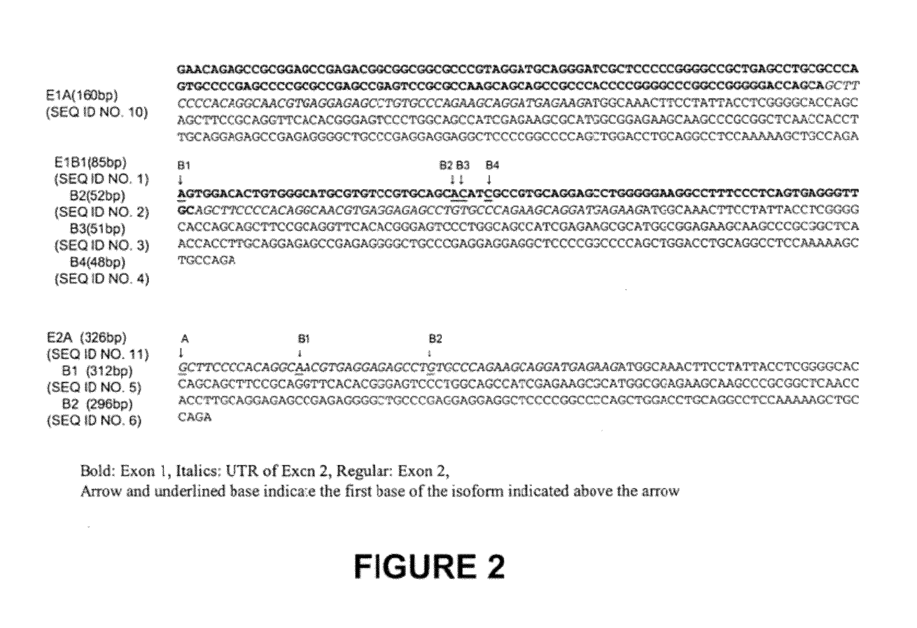

The E1B1, E1B2, E1B3, E1B4, E2B1, and E2B2 splice variants are from the 5′ region, the locations of which in the SCN5A gene and mRNA are depicted in FIG. 1. The nucleic acid sequences for E1B1, E1B2, E1B3, E1B4, E2B1, and E2B2 are shown in FIG. 2. E1A (SEQ ID NO. 10) is the wild-type (or full-length) isoform in and/or near the 5′UTR of exon 1, while E1B1, E1B2, E1B3, E1B4 are its various spliced variants. Similarly, E2A (SEQ ID NO. 11) is the wild-type (full-length) isoform in and/or near the 5′UTR of exon 2, while E2B1, and E2B2 are its various variants.

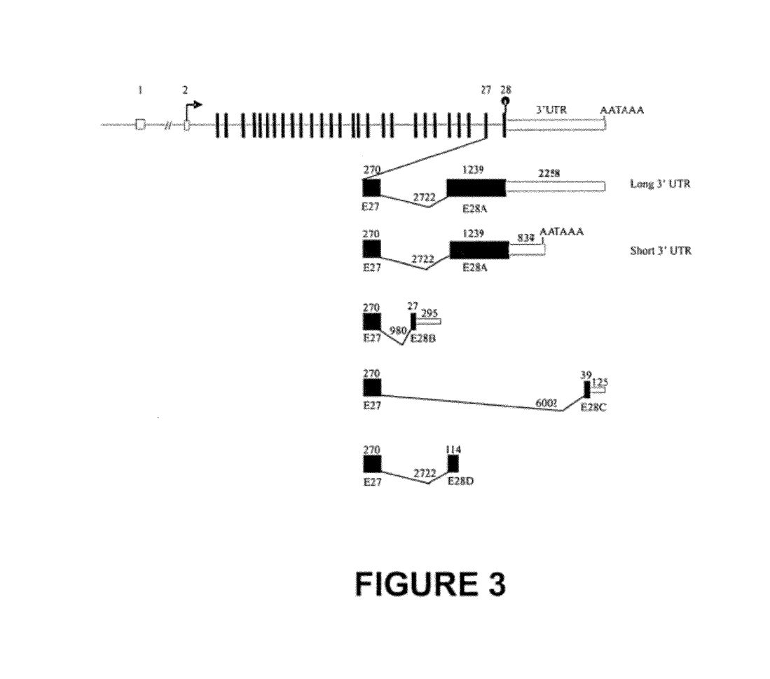

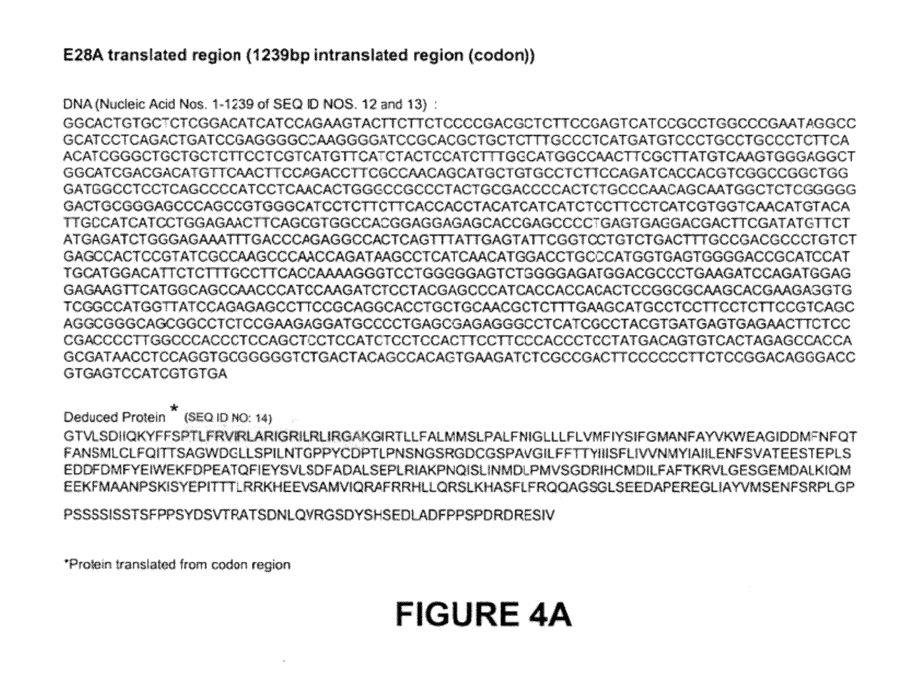

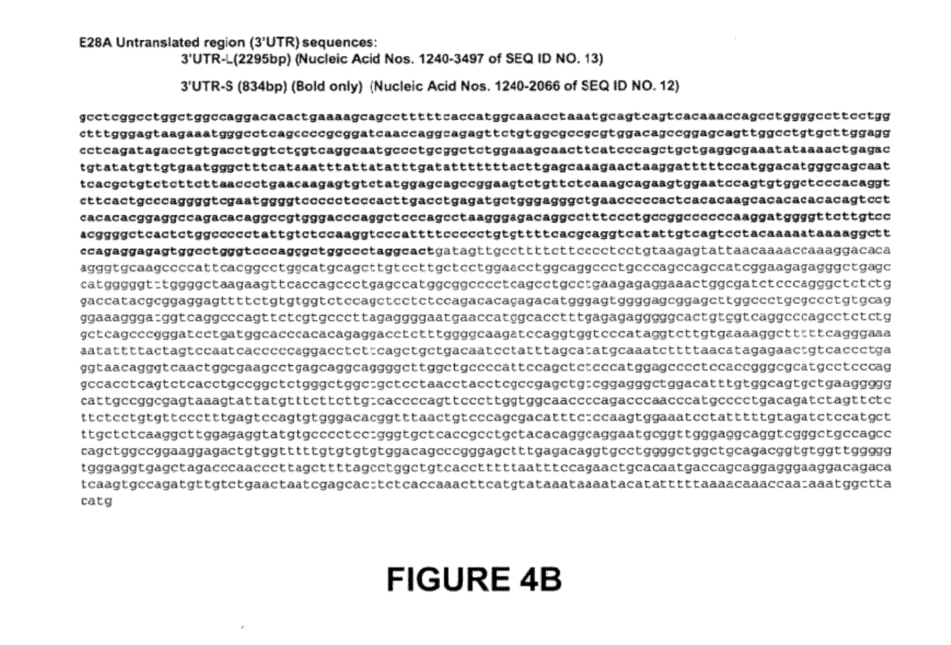

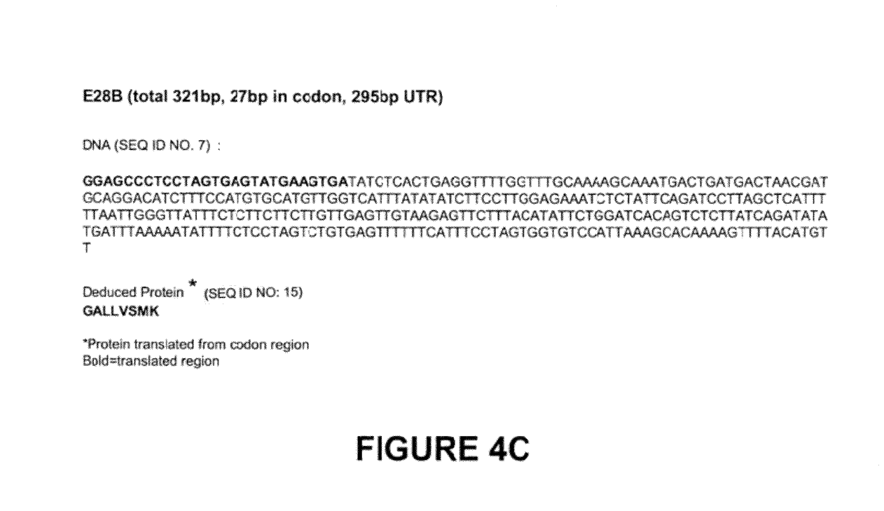

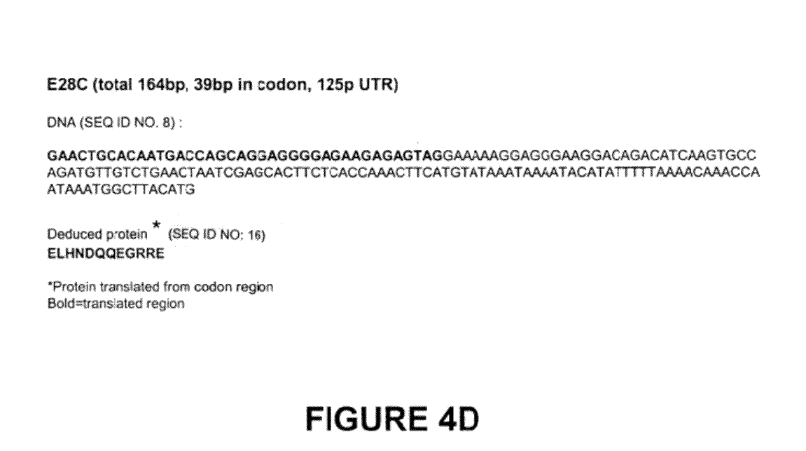

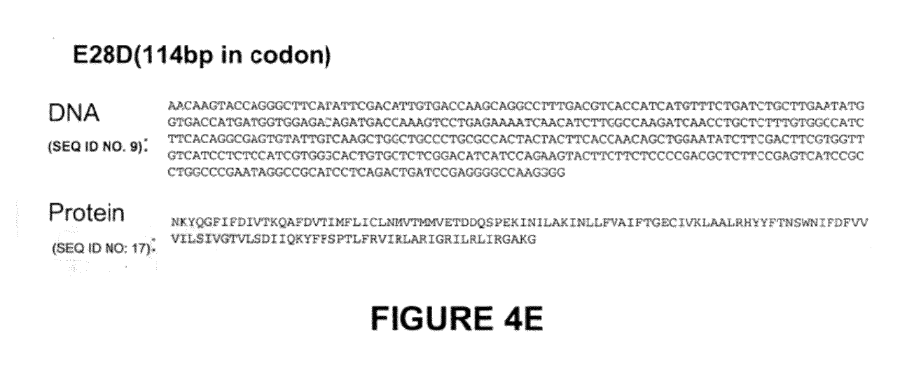

The E28B, E28C, or E28D splice variants are from or near the 3′ untranslated region, the locations of which in the SCN5A mRNA are depicted in FIG. 3. The nucleic acid sequences for E28B, E28C, and E28D are set forth in SEQ ID NOs: 7-9, respectively. E28A is the wild-type (or full-length) isoform of the 3′ region of exon 28, while E28B, E28C, or E28D are its various truncated splice variant encoding shortened, dysfunctional channels. There are two isoforms of the E28A: E28A-short (E28A-S) (SEQ ID NO. 12) and E28A-long (E28A-L) (SEQ ID NO. 13). Both isoforms of E28A contains 1239 base pairs in the translated region. The difference between E28-L and E28-S resides in the UTR where E28A-L contains 2295 base pairs of the 3′UTR, while E28A-S contains 834 base pairs of the 3′UTR. E28A-S contains only the first 834 base pairs of the 3′UTR. Both E28B and E28C contains untranslated and translated regions while E28D contains only translated region of exon 28.

The physiological significance of the E28B, E28C and E28D splice variants is supported by a premature stop codon in exon 28 of one of the two SCN5A alleles, resulting in an 86% reduction in the Na⁺ current (Shang et al., Circ. Res., 101:1146-1154, 2007).

Inhibiting Downregulation of Full-Length SCN5a Via Inhibition of hLuc7a, RBM25 and/or PERK

In one aspect, described herein is a method of inhibiting downregulation of full-length SCN5A (e.g., E28A) in a cell comprising contacting the cell with a compound that inhibits the activity of splicing factor hLuc7a, splicing factor RBM25 and/or PERK in an amount effective to inhibit down regulation of full-length SCN5A. In some embodiments, the cell is a blood cell, a muscle cell or a neuron. In other embodiments, the cell is a leukocyte, a macrophage or a cardiac cell. In some embodiments, the cell is an in vivo cell and the method is an in vivo method. Accordingly, in exemplary aspects, the compound is administered to the body in which the cell resides. In specific aspects, the compound is administered to a subject, e.g., a mammal (e.g., a human), in need for inhibited downregulation of full-length SCN5A. In exemplary aspects, the subject is an individual at risk of or suffering from arrhythmia or an individual at risk of or suffering from heart failure.

In one embodiment, the method inhibits or reverses an effect of hypoxia or angiotensin II in the cell. In another embodiment, downregulation of splicing factor hLuc7A or splicing factor RBM25 downregulates expression of abnormal SCN5A splice variants. In another embodiment, downregulation of PERK upregulates expression of full-length SCN5A.

It has been shown that RBM25 is a splicing factor that binds tightly to the canonical RNA sequence CGGGC(A) (Zhou et al., Mol. Cell. Biol., 28:5924-5936, 2008). Therefore, in some embodiments, the compound that inhibits activity of splicing factor hLuc7a, splicing factor RBM25 and/or PERK reduces splicing factor interaction with the canonical sequence CGGGCA in a genomic polynucleotide encoding SCN5A.

Any compound that inhibits the activity of hLuc7a, RBM25 and/or PERK is contemplated for use in the methods described herein. In one embodiment, the compound includes inhibitor oligonucleotides or polynucleotides, including pharmaceutically acceptable salts thereof, e.g., sodium salts. Nonlimiting examples include antisense oligonucleotides (Eckstein, Antisense Nucleic Acid Drug Dev., 10: 117-121 (2000); Crooke, Methods Enzymol., 313: 3-45 (2000); Guvakova et al., J. Biol. Chem., 270: 2620-2627 (1995); Manoharan, Biochim. Biophys. Acta, 1489: 117-130 (1999); Baker et al., J. Biol. Chem., 272: 11994-12000 (1997); Kurreck, Eur. J. Biochem., 270: 1628-1644 (2003); Sierakowska et al., Proc. Natl. Acad. Sci. USA, 93: 12840-12844 (1996); Marwick, J. Am. Med. Assoc., 280: 871 (1998); Tomita and Morishita, Curr. Pharm. Des., 10: 797-803 (2004); Gleave and Monia, Nat. Rev. Cancer, 5: 468-479 (2005) and Patil, AAPS J., 7: E61-E77 (2005)), triplex oligonucleotides (Francois et al., Nucleic Acids Res., 16: 11431-11440 (1988) and Moser and Dervan, Science, 238: 645-650 (1987)), ribozymes/deoxyribozymes (DNAzymes) (Kruger et al., Tetrahymena. Cell, 31: 147-157 (1982); Uhlenbeck, Nature, 328: 596-600 (1987); Sigurdsson and Eckstein, Trends Biotechnol., 13: 286-289 (1995); Kumar et al., Gene Ther., 12: 1486-1493 (2005); Breaker and Joyce, Chem. Biol., 1: 223-229 (1994); Khachigian, Curr. Pharm. Biotechnol., 5: 337-339 (2004); Khachigian, Biochem. Pharmacol., 68: 1023-1025 (2004) and Trulzsch and Wood, J. Neurochem., 88: 257-265 (2004)), small-interfering RNAs/RNAi (Fire et al., Nature, 391: 806-811 (1998); Montgomery et al., Proc. Natl. Acad. Sci. U.S.A., 95: 15502-15507 (1998); Cullen, Nat. Immunol., 3: 597-599 (2002); Hannon, Nature, 418: 244-251 (2002); Bernstein et al., Nature, 409: 363-366 (2001); Nykanen et al., Cell, 107: 309-321 (2001); Gilmore et al., J. Drug Target., 12: 315-340 (2004); Reynolds et al., Nat. Biotechnol., 22: 326-330 (2004); Soutschek et al., Nature, 432173-178 (2004); Ralph et al., Nat. Med., 11: 429-433 (2005); Xia et al., Nat. Med., 10816-820 (2004) and Miller et al., Nucleic Acids Res., 32: 661-668 (2004)), aptamers (Ellington and Szostak, Nature, 346: 818-822 (1990); Doudna et al., Proc. Natl. Acad. Sci. U.S.A., 92: 2355-2359 (1995); Tuerk and Gold, Science, 249: 505-510 (1990); White et al., Mol. Ther., 4: 567-573 (2001); Rusconi et al., Nature, 419: 90-94 (2002); Nimjee et al., Mol. Ther., 14: 408-415 (2006); Gragoudas et al., N. Engl. J. Med., 351: 3805-2816 (2004); Vinores, Curr. Opin. Mol. Ther., 5673-679 (2003) and Kourlas and Schiller et al., Clin. Ther., 28: 36-44 (2006)) or decoy oligonucleotides (Morishita et al., Proc. Natl. Acad. Sci. U.S.A., 92: 5855-5859 (1995); Alexander et al., J. Am. Med. Assoc., 294: 2446-2454 (2005); Mann and Dzau, J. Clin. Invest., 106: 1071-1075 (2000) and Nimjee et al., Annu. Rev. Med., 56: 555-583 (2005)). The foregoing documents are hereby incorporated by reference in their entirety herein, with particular emphasis on those sections of the documents relating to methods of designing, making and using inhibitory oligonucleotides. Commercial providers such as Ambion Inc. (Austin, Tex.), Darmacon Inc. (Lafayette, Colo.), InvivoGen (San Diego, Calif.), and Molecular Research Laboratories, LLC (Herndon, Va.) generate custom siRNA molecules. In addition, commercial kits are available to produce custom siRNA molecules, such as SILENCER™ siRNA Construction Kit (Ambion Inc., Austin, Tex.) or psiRNA System (InvivoGen, San Diego, Calif.).

Inhibitory oligonucleotides which are stable, have a high resistance to nucleases, possess suitable pharmacokinetics to allow them to traffic to target tissue site at non-toxic doses, and have the ability to cross through plasma membranes are contemplated for use as a therapeutic. In some embodiments, inhibitory oligonucleotides are complementary to the coding portion of a target gene, 3′ or 5′ untranslated regions, or intronic sequences in a gene, or alternatively coding or intron sequences in the target mRNA. Intron sequences are generally less conserved and thus may provide greater specificity. In one embodiment, the inhibitory oligonucleotide inhibits expression of a gene product of one species but not its homologue in another species; in other embodiments, the inhibitory oligonucleotide inhibits expression of a gene in two species, e.g. human and primate, or human and murine.

The constitutive expression of antisense oligonucleotides in cells has been shown to inhibit gene expression, possibly via the blockage of translation or prevention of splicing. In certain embodiments, the inhibitory oligonucleotide is capable of hybridizing to at least 8, 9, 10, 11, or 12 consecutive bases of the hLuc7A, RBM25 and/or PERK genes or mRNA (or the reverse strand thereof) under moderate or high stringency conditions. In some embodiments, suitable inhibitory oligonucleotides are single stranded and contain a segment, e.g. at least 12, 13, 14, 15, 16, 17 or 18 bases in length, that is sufficiently complementary to, and specific for, an mRNA or DNA molecule such that it hybridizes to the mRNA or DNA molecule and inhibits transcription, splicing or translation. Generally complementarity over a length of less than 30 bases is more than sufficient.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short nucleic acids (e.g., 10 to 50 nucleotides) and at least about 60° C. for longer nucleic acids (e.g., greater than 50 nucleotides). Stringent conditions may also be achieved with the addition of destabilizing agents such as formamide. Exemplary low stringency conditions include hybridization with a buffer solution of 30% to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50° C. to 55° C. Exemplary moderate stringency conditions include hybridization in 40% to 45% formamide, 1.0 M NaCl, 1% SDS at 37° C., and a wash in 0.5× to 1×SSC at 55° C. to 60° C. Exemplary high stringency conditions include hybridization in 50% formamide, 1 M NaCl, 1% SDS at 37° C., and a wash in 0.1×SSC at 60° C. to 65° C. Duration of hybridization is generally less than about 24 hours, usually about 4 hours to about 12 hours.

In some cases, depending on the length of the complementary region, one, two or more mismatches are tolerated without affecting inhibitory function. In certain embodiments, the inhibitory oligonucleotide is an antisense oligonucleotide, an inhibitory RNA (including siRNA or RNAi, or shRNA), a DNA enzyme, a ribozyme (optionally a hammerhead ribozyme), an aptamer, or pharmaceutically acceptable salts thereof. In one embodiment, the oligonucleotide targets the nucleotides located in the vicinity of the 3′ untranslated region of the SCN5A mRNA. In one embodiment, the oligonucleotide is complementary to at least 10 bases of the hLuc7A mRNA sequence (Genbank Accession Nos. NM_{—}006107 and NM_{—}016424), the RBM25 mRNA sequence (Genbank Accession No.: NM_{—}021239) or a PERK mRNA sequence (including, but not limited to, Genbank Accession Nos: NM_{—}003094.2, NM_{—}004320.3, NM_{—}0173201.2, NM_{—}203463.1, NM_{—}006850.2, NM_{—}003329.2, NM_{—}181339.1, NM_{—}006260.3, NG_{—}016424.1, NM_{—}004836.5, NM_{—}001122752.1, NM_{—}005025.4, NM_{—}033266.3, NM_{—}032025.3, NM_{—}005130.3, NM_{—}001013703.2, BC126356.1 and BC126354.1).

The specific sequence utilized in design of the oligonucleotides may be any contiguous sequence of nucleotides contained within the expressed gene message of the target. The worker of ordinary skill in the art will readily appreciate sequences that can be targeted for inhibition based on the full-length hLuc7A (Genbank Accession Nos. NM_{—}006107 and NM_{—}016424), RBM25 (Genbank Accession No.: NM_{—}021239) and PERK mRNA sequence (including, but not limited to, Genbank Accession Nos: NM_{—}003094.2, NM_{—}004320.3, NM_{—}0173201.2, NM_{—}203463.1, NM_{—}006850.2, NM_{—}003329.2, NM_{—}181339.1, NM_{—}006260.3, NG_{—}016424.1, NM_{—}004836.5, NM_{—}001122752.1, NM_{—}005025.4, NM_{—}033266.3, NM_{—}032025.3, NM_{—}005130.3, NM_{—}001013703.2, BC126356.1 and BC126354.1) sequences known in the art. Factors that govern a target site for the inhibitory oligonucleotide sequence include the length of the oligonucleotide, binding affinity, and accessibility of the target sequence. In some embodiments, sequences are screened in vitro for potency of their inhibitory activity by measuring inhibition of target protein translation and target related phenotype, e.g., inhibition of cell proliferation in cells in culture. In general it is known that most regions of the RNA (5′ and 3′ untranslated regions, AUG initiation, coding, splice junctions and introns) can be targeted using antisense oligonucleotides. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference in its entirety.

Short interfering (si) RNA technology (also known as RNAi) generally involves degradation of an mRNA of a particular sequence induced by double-stranded RNA (dsRNA) that is homologous to that sequence, thereby “interfering” with expression of the corresponding gene. Any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Accordingly, siRNA may be affected by introduction or expression of relatively short homologous dsRNAs. Exemplary siRNAs have sense and antisense strands of about 21 nucleotides that form approximately 19 nucleotides of double stranded RNA with overhangs of two nucleotides at each 3′ end. Indeed the use of relatively short homologous dsRNAs may have certain advantages.

The double stranded oligonucleotides used to effect RNAi are preferably less than 30 base pairs in length, for example, about 25, 24, 23, 22, 21, 20, 19, 18, or 17 base pairs or less in length, and contain a segment sufficiently complementary to the target mRNA to allow hybridization to the target mRNA. Optionally the dsRNA oligonucleotides may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al., supra). Exemplary dsRNAs may be synthesized chemically or produced in vitro or in vivo using appropriate expression vectors (see, e.g., Elbashir et al., Genes Dev., 15:188-200 (2001)). Longer RNAs may be transcribed from promoters, such as T7 RNA polymerase promoters, known in the art.

Longer dsRNAs of 50, 75, 100, or even 500 base pairs or more also may be utilized in certain embodiments of the invention. Exemplary concentrations of dsRNAs for effecting RNAi are about 0.05 nM, 0.1 nM, 0.5 nM, 1.0 nM, 1.5 nM, 25 nM, or 100 nM, although other concentrations may be utilized depending upon the nature of the cells treated, the gene target and other factors readily discernable to the skilled artisan.

Further compositions, methods and applications of siRNA technology are provided in U.S. Pat. Nos. 6,278,039; 5,723,750; and 5,244,805, which are incorporated herein by reference in its entirety.

shRNA may comprise sequences that were selected at random, or according to any rational design selection procedure. For example, rational design algorithms are described in International Patent Publication No. WO 2004/045543 and U.S. Patent Publication No. 20050255487, the disclosures of which are incorporated herein by reference in their entireties. Additionally, it may be desirable to select sequences in whole or in part based on average internal stability profiles (“AISPs”) or regional internal stability profiles (“RISPs”) that may facilitate access or processing by cellular machinery.

Ribozymes are enzymatic RNA molecules capable of catalyzing specific cleavage of mRNA, thus preventing translation. (For a review, see Rossi, Current Biology, 4:469-471 (1994)). The mechanism of ribozyme action involves sequence specific hybridization of the ribozyme molecule to complementary target RNA, followed by an endonucleolytic cleavage event. The ribozyme molecules preferably include (1) one or more sequences complementary to a target mRNA, and (2) the well known catalytic sequence responsible for mRNA cleavage or a functionally equivalent sequence (see, e.g., U.S. Pat. No. 5,093,246, which is incorporated herein by reference in its entirety).

Gene targeting ribozymes may contain a hybridizing region complementary to two regions of a target mRNA, each of which is at least 5, 6, 7, 8, 9, 10, 11, 12, 13, 14, 15, 16, 17, 18, 19, or 20 contiguous nucleotides (but which need not both be the same length).

Ribozymes for use in a method described herein also include RNA endoribonucleases (“Czech-type ribozymes”) such as the one which occurs naturally in Tetrahymena thermophila (known as the IVS, or L-19 IVS RNA) and which has been extensively described in Zaug et al., Science, 224:574-578 (1984); Zaug, et al., Science, 231:470-475 (1986); Zaug et al., Nature, 324:429-433 (1986); International Patent Publication No. WO 88/04300; and Been et al., Cell, 47:207-216 (1986)). The Cech-type ribozymes have an eight base pair active site which hybridizes to a target RNA sequence whereafter cleavage of the target RNA takes place. In one embodiment, the inventive method employs those Cech-type ribozymes which target eight base-pair active site sequences that are present in a target gene or nucleic acid sequence.

Alternatively, target gene expression can be reduced by targeting deoxyribonucleotide sequences complementary to the regulatory region of the gene (i.e., the promoter and/or enhancers) to form triple helical structures that prevent transcription of the gene in target cells in the body. (See generally Helene, C., Anticancer Drug Des., 6:569-84 (1991); Helene et al., Ann. N.Y. Acad. Sci., 660:27-36 (1992); and Maher, L. J., Bioassays, 14:807-15 (1992)).

Alternatively, DNA enzymes may be used to inhibit expression of target gene, such as the sclerostin gene. DNA enzymes incorporate some of the mechanistic features of both antisense and ribozyme technologies. DNA enzymes are designed so that they recognize a particular target nucleic acid sequence, much like an antisense oligonucleotide. They are, however, also catalytic and specifically cleave the target nucleic acid.

DNA enzymes include two basic types identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Methods of making and administering DNA enzymes can be found, for example, in U.S. Pat. No. 6,110,462. Additionally, one of skill in the art will recognize that, like antisense oligonucleotide, DNA enzymes can be optionally modified to improve stability and improve resistance to degradation.

Inhibitory oligonucleotides can be administered directly or delivered to cells by transformation or transfection via a vector, including viral vectors or plasmids, into which has been placed DNA encoding the inhibitory oligonucleotide with the appropriate regulatory sequences, including a promoter, to result in expression of the inhibitory oligonucleotide in the desired cell. Known methods include standard transient transfection, stable transfection and delivery using viruses ranging from retroviruses to adenoviruses. Delivery of nucleic acid inhibitors by replicating or replication-deficient vectors is contemplated. Expression can also be driven by either constitutive or inducible promoter systems (Paddison et al., Methods Mol. Biol., 265:85-100 (2004)). In other embodiments, expression may be under the control of tissue or development-specific promoters. For example, in one embodiment, the promoter sequence comprises a cardiac-specific or skeletal muscle-specific promoter.

For example, vectors may be introduced by transfection using carrier compositions such as Lipofectamine 2000 (Life Technologies) or Oligofectamine (Life Technologies). Transfection efficiency may be checked using fluorescence microscopy for mammalian cell lines after co-transfection of hGFP-encoding pAD3 (Kehlenback et al., J. Cell Biol., 141:863-74 (1998)).

The delivery route will be the one that provides the best inhibitory effect as measured according to the criteria described above. Delivery mediated by cationic liposomes, delivery by retroviral vectors and direct delivery are efficient.

The effectiveness of the inhibitory oligonucleotide may be assessed by any of a number of assays, including reverse transcriptase polymerase chain reaction or Northern blot analysis to determine the level of existing SCN5A splice variants mRNA.

In another embodiment, the compound that inhibits the activity of hLuc7a, RBM25 and/or PERK is an antibody. The term “antibody” is used in the broadest sense and includes fully-assembled antibodies, monoclonal antibodies, polyclonal antibodies, multispecific antibodies (including bispecific antibodies), chimeric antibodies, human antibodies, humanized antibodies, antibody fragments that can bind an antigen (including, Fab′, F′(ab)₂, Fv, single chain antibodies, diabodies), and recombinant peptides comprising the foregoing as long as they exhibit the desired biological activity. Multimers or aggregates of intact antibodies and/or fragments, including chemically derivatized antibodies, are contemplated. Antibodies of any isotype class or subclass, including IgG, IgM, IgD, IgA, and IgE, IgG1, IgG2, IgG3, IgG4, IgA1 and IgA2, or any allotype, are contemplated. Standard techniques are employed to generate polyclonal or monoclonal antibodies directed against hLuc7a, RBM25 and/or PERK and to generate useful antigen-binding fragments thereof or variants thereof. Such protocols can be found, for example, in Sambrook et al., Molecular Cloning: a Laboratory Manual. Second Edition, Cold Spring Harbor, N.Y.: Cold Spring Harbor Laboratory (1989); Harlow et al. (Eds), Antibodies A Laboratory Manual; Cold Spring Harbor Laboratory; Cold Spring Harbor, N.Y. (1988). Peptibodies are also contemplated. The term “peptibody” refers to a molecule comprising an antibody Fc domain attached to at least one peptide, which has specific binding properties. The production of peptibodies is generally described in PCT publication WO 00/24782, the disclosure of which is incorporated herein by reference.

Small molecules that inhibit the activity of hLuc7a, RBM25 and/or PERK are also contemplated. The small molecule, in some embodiments, is a compound that acts directly or indirectly on exon 28 of the SCN5A gene (without interfering with transcription of full-length SCN5A), hLuc7a, RBM25 and or PERK or that decreases the level of at least one of hypoxia and/or angiotensin II in vivo. The term “small molecule” includes a compound or molecular complex, either synthetic, naturally derived, or partially synthetic, and which preferably has a molecular weight of less than 5,000 Daltons (e.g., between about 100 and 1,500 Daltons). Agents can be obtained using any of the numerous approaches in combinatorial library methods known in the art, including spatially addressable parallel solid phase or solution phase libraries, synthetic library methods requiring deconvolution, the “one-bead one-compound” library method, and synthetic library methods using affinity chromatography selection (see, e.g., Lam, Anticancer Drug Des., 12:145 (1997) and U.S. Pat. Nos. 5,738,996; 5,807,683; and 7,261,892).

### Assaying for Modulators of the Expression of Abnormal SCN5A Splice Variants

Methods for identifying modulators of the expression of abnormal splice variants are also provided. In one aspect, described herein is a method of identifying a compound that inhibits expression of an abnormal SCN5A splice variant in a cell comprising the step of determining a level of a SCN5A splice variant in the cell in the presence and absence of a test compound, wherein a reduced level of the splice variant in the presence of the test compound compared to the level of the splice variant in the absence of the test compound identifies the test compound as an inhibitor of the expression of a SCN5A splice variant. Such screening techniques are useful in the general identification of a compound that will inhibit abnormal splicing of SCN5A in a cell, with such compounds being useful as therapeutic agents.

In another aspect, described herein is a method of identifying a compound that inhibits transcription of an abnormal SCN5A splice variant in a cell comprising the step of contacting the cell that comprises a SCN5A gene with a test compound in the presence and absence of a splicing factor selected from the group consisting of hLuc7a and RBM25, wherein the splicing factor binds to the SCN5A gene in the absence of the test compound, and determining the presence or absence of an abnormal splice variant in the cell, wherein the absence of the abnormal splice variant in the cell identifies the test compound as a compound that inhibits transcription of an abnormal splice variant.

In some embodiments, the abnormal SCNA splice variant is selected from the group consisting of E28B (SEQ ID NO: 7), E28C (SEQ ID NO: 8) and E28D (SEQ ID NO: 9).

In some embodiments, the test compound is an antisense oligonucleotide, an antibody or small molecule as described elsewhere herein.

The test compounds can be screened for their ability to promote splicing or inhibit splicing. RNA splicing can be detected by any of a variety of assays that detect the presence or absence of an exon in an RNA, including, without limitation, detection of protein domains encoded by particular exons (or introduced into particular exons) in translated proteins, electrophoretic separation and gel analysis of RNA or protein, polymerase chain reaction-based assays, Northern analysis or RNase protection using exon-specific probes, invasion cleavage assay (E is et al. Nature Biotechnol. 19: 673-676 (2001), radionucleotide or fluorescently labeled nucleotide incorporation, etc.

### Therapeutic Methods and Methods of Monitoring Efficacy of Treatment

Both prophylactic and therapeutic methods of treating a subject at risk of (or susceptible to) a disorder or having a disorder associated with aberrant or unwanted target gene expression or activity (e.g., in exemplary embodiments, expression of abnormal SCN5A splice variants) is provided. “Treatment”, or “treating” as used herein, means the application or administration of a therapeutic agent (e.g., an oligonucleotide, an antibody or a small molecule) or vector or transgene encoding same, etc.) to a subject or application or administration of a therapeutic agent to an isolated tissue (including, but not limited to, cardiac tissue) or cells (including, but not limited to, cardiac cells) from a subject, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one embodiment, a prophylactic method of treating arrhythmia in a subject is provided, the method comprising identifying the subject as being at risk for developing arrhythmia and administering to a subject at risk of arrhythmia a compound that inhibits the activity of hLuc7a, RBM25 and/or PERK in an amount effective to prevent arrhythmia. In some embodiment, the method alternatively comprises the step of identifying an individual at risk of arrhythmia. In some embodiments, the method comprises identifying a subject at risk for developing arrhythmia. In such embodiments, the identifying comprises screening for the presence of an abnormal SCN5A splice variant in a biological sample of the subject, wherein the presence of the abnormal splice variant identifies the subject as being at risk for developing arrhythmia. For example, the presence of one or more of the SCNA splice variants E28B (SEQ ID NO: 7), E28C (SEQ ID NO: 8) and/or E28D (SEQ ID NO: 9) identifies in the biological sample identifies the subject as being at risk for developing arrhythmia. The screening step, in some embodiments, comprises obtaining a biological sample from the subject and analyzing nucleic acid from the sample for the presence of an abnormal splice variant. As used herein, the term “arrhythmia,” also known as “cardiac arrhythmia,” refers to a heterogenous group of conditions affecting the electrical behavior of the heart, e.g., a heart beat that is too fast (“tachycardias”), too slow (“bradycardias”) or of irregular pattern. It will be appreciated that arrhythmias may be classified based on their rate (normal, tachycardia, bradycardia), their mechanism (automaticity, re-entry, fibrillation), or by their site of origin (atrial, ventricular, junctional, or atrio-ventricular).

In some embodiments, the identifying step comprises determining a level of hLuc7a, RBM25 and/or PERK in a biological sample (e.g., white blood cell or cardiac tissue) of a subject, wherein an increase in the level of hLuc7a, RBM25 and/or PERK in the sample identifies the subject as being at risk for developing arrhythmia.

Methods are also provided wherein the biological sample is blood or cardiac tissue. In some embodiments, the biological sample is blood and white blood cells in the blood are analyzed for the presence of an abnormal SCN5A splice variant.

Methods of treating arrhythmia in a subject is also provided. For example, in one embodiment, the method comprises administering an effective amount of a compound that that inhibits activity of splicing factor hLuc7a, splicing factor RBM25 and/or PERK to the subject.

In some embodiments, the subject is human. Practice of methods of the invention in other mammalian subjects, especially mammals that are conventionally used as models for demonstrating therapeutic efficacy in humans (e.g., primate, porcine, canine, or rabbit animals), is also contemplated. In some embodiments, the subject is suffering from a cardiac disorder, including but not limited to, heart failure, ischemia, myocardial infarction, congestive heart failure, arrhythmia, transplant rejection and the like. In one embodiment, the subject is suffering from heart failure. In another embodiment, the subject is suffering from arrhythmia.

Methods are also provided to monitor the efficacy of treatment. In such embodiments, the method comprises determining a level of hLuc7a, RBM25 and/or PERK in a biological sample (e.g., white blood cell or cardiac tissue) of a subject before and after treatment with a compound that inhibits activity of hLuc7a, RBM25 and/or PERK. In such a method, a change in the level of hLuc7 am RBM25 and/or PERK in the sample taken after treatment compared to the level of hLuc7am RBM25 and/or PERK in the sample taken before treatment indicates efficacy of the treatment. In some embodiments, a first biological sample is obtained from the subject to be treated prior to initiation of therapy or part way through a therapy regime. Alternatively, in some embodiments, a first biological sample is obtained from a subject known not to suffer from a condition being treated. In some embodiments, the second biological sample is obtained in a similar manner, but at a time following onset of therapy. The second biological sample, in some embodiments, is obtained at the completion of, or part way through therapy, provided that at least a portion of therapy takes place between the isolation of the first and second biological samples. A decrease in the level of hLuc7a, RBM25 and/or PERK in the second biological sample (e.g., post-treatment) compared to the level of hLuc7a, RBM25 and/or PERK in the first biological sample (e.g., prior to treatment or from an subject known not to suffer from the condition being treated) indicates a degree of effective therapy.

The level of abnormal SCN5A splice variants in a biological sample, in some embodiments, is analyzed in a similar manner to monitor efficacy of treatment, with a decrease in the level of abnormal SCN5A splice variants in the sample indicates effective therapy. In such embodiments, the method to monitor efficacy of treatment comprises determining a level of abnormal SCN5A splice variants in a biological sample of a subject before and after treatment with a compound that inhibits activity of hLuc7a, RBM25 and/or PERK. In such a method, a change in the level of abnormal SCN5A splice variants in the sample taken after treatment compared to the level of abnormal SCN5A splice variant in the sample before treatment indicates efficacy of treatment. A decrease in the level of abnormal SCN5A splice variants in the second biological sample (e.g., post-treatment) compared to the level of abnormal SCN5A splice variants in the first biological sample (e.g., prior to treatment or from an individual known not to suffer from the condition being treated) indicates a degree of effective therapy.

Alternatively, the level of full-length SCN5A in a biological sample post-treatment is analyzed to monitor efficacy of treatment, with an increase in the level of full-length SCN5A in the sample indicates effective therapy. In such embodiments, the method to monitor efficacy of treatment comprises determining a level of full-length SCN5A in a biological sample of a subject before and after treatment with a compound that inhibits activity of hLuc7a, RBM25 and/or PERK. In such a method, a change in the level of full-length SCN5A in the sample taken after treatment compared to the level of full-length SCN5A in the sample before treatment indicates efficacy of treatment. An increase in the level of full-length SCN5A in the second biological sample (e.g., post-treatment) compared to the level of full-length SCN5A in the first biological sample (e.g., prior to treatment or from an individual known not to suffer from the condition being treated) indicates a degree of effective therapy.

In yet another alternative, a level of hypoxia and/or angiotensin II in a biological sample post-treatment is analyzed to monitor efficacy of treatment, with a decrease in the level of hypoxia and/or angiotensin II in the sample indicates effective therapy. In such embodiments, the method to monitor efficacy of treatment comprises determining a level of hypoxia or angiotensin II in a biological sample of a subject before and after treatment with a compound that inhibits activity of hLuc7a, RBM25 and/or PERK. In such a method, a change in the level of hypoxia or angiotensin II in the sample taken after treatment compared to the level of hypoxia or angiotensin II in the sample before treatment indicates efficacy of treatment. A decrease in the level of hypoxia or angiotensin II in the second biological sample (e.g., post-treatment) compared to the level of hypoxia or angiotensin II in the first biological sample (e.g., prior to treatment or from an individual known not to suffer from the condition being treated) indicates a degree of effective therapy.

### Combination Therapy

In some embodiments, the prophylactic and therapeutic methods described herein are used in combination with standard of care therapeutics of heart failure including, but not limited to, diuretics, inotropes, coronary vasodilators and beta blockers or conventional therapeutics of circulatory diseases such as hypertension (e.g. angiotensin converting enzyme (ACE) inhibitors, angiotensin receptor blockers (ARBs) and/or calcium channel blockers), either simultaneously or at different times. Diuretics are generally used for relief of congestive symptoms and help the kidneys rid the body of excess fluid, thereby reducing blood volume and the heart's workload. Diuretics can include, but are not limited to loop diuretics (e.g. furosemide, bumetanide); thiazide diuretics (e.g. hydrochlorothiazide, chlorthalidone, chlorthiazide); potassium-sparing diuretics (e.g. amiloride); spironolactone and eplerenone. Inotropes, such as a cardiac glycoside, a beta-adrenergic agonist or a phosphodiesterase inhibitor, strengthen the heart's pumping action in patients with low cardiac output; inotropes can include but are not limited to digoxin, dobutamine, milrinone, istaroxime, omecamtiv mecarbil. Vasodilators, cause the peripheral arteries to dilate, making it easier for blood to flow; examples of vasodilators include, but are not limited, nitroglycerin, nitorprusside, and neseritide. Activation of neurohormonal systems that include the renin-andiotensin-aldosterone system (RAAS) and the sympathetic nervous system also contribute to the pathophysiology of heart failure. Drugs that inhibit activation of RAAS fall into three major categories: ACE inhibitors (including but not limited to ramipril, enalapril, and captopril), ARBs (including but not limited to valsarten, candesarten, irbesarten and losarten), and aldosterone receptor blockers (e.g., spironolactone and eplerenone.) Beta blockers counter the effects of activation of the sympathetic nervous system and slow the heart rate by blocking the effects of adrenalin; beta blockers include, but are not limited to carvedilol, metoprolol, bisoprolol, atenolol, propranolol, timolol and bucindolol.

In various aspects, the compound that inhibits activity of hLuc7a, RBM25 and/or PERK and standard of care therapeutic are administered concurrently or sequentially. In embodiments where the compound that inhibits activity of hLuc7a, RBM25 and/or PERK and standard of care therapeutic are administered separately, one would generally ensure that a significant period of time did not expire between the times of each delivery, such that the compound and standard of care therapeutic(s) would still be able to exert an advantageously combined effect. In such instances, it is contemplated that both modalities would be administered within about 12-24 hours of each other. In some situations, it may be desirable to extend the time period for treatment significantly, from several days (2, 3, 4, 5, 6 or 7) to several weeks (1, 2, 3, 4, 5, 6, 7 or 8). Repeated treatments with one or both agents is specifically contemplated.

### Compositions and Formulations

Compositions for use in accordance with the present invention may be formulated in a conventional manner using one or more physiologically acceptable carriers comprising excipients and auxiliaries which facilitate processing of a therapeutic composition into preparations which can be used pharmaceutically. “Therapeutic compositions” or “therapeutic compound” as used herein refers to a composition comprising a therapeutic compound that inhibits activity of splicing factor hLuc7a, splicing factor RBM25 an/or PERK.

The therapeutic compound may also be admixed, encapsulated, conjugated or otherwise associated with other molecules, molecule structures or mixtures of compounds, as for example, liposomes, carriers, diluents, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

In embodiments where the therapeutic is an antisense compound, such antisense compound encompass any pharmaceutically acceptable salts, esters, or salts of such esters, or any other compound which, upon administration to an animal, including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE ((S acetyl-2-thioethyl) phosphate) derivatives according to the methods disclosed in WO 93/24510 to Gosselin et al., published Dec. 9, 1993 or in WO 94/26764 and U.S. Pat. No. 5,770,713 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto. For oligonucleotides, preferred examples of pharmaceutically acceptable salts and their uses are further described in U.S. Pat. No. 6,287,860, which is incorporated herein in its entirety.

Compositions comprising the therapeutic compound may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions useful in the present invention include, but are not limited to, solutions, emulsions, foams and liposome-, micelle-, or nanoparticle-containing formulations. The pharmaceutical compositions and formulations of the present invention may comprise one or more penetration enhancers, carriers, excipients, diluents, or other active or inactive ingredients. Emulsions and their uses are well known in the art and are further described in U.S. Pat. No. 6,287,860, which is incorporated herein in its entirety.

Formulations useful in the present invention include liposomal formulations. As used in the present invention, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH sensitive or negatively charged are believed to entrap DNA rather than complex with it. Both cationic and noncationic liposomes have been used to deliver DNA to cells, and can be used to deliver compounds of the invention.

One of skill in the art will recognize that formulations are routinely designed according to their intended use, i.e. route of administration.

In some embodiments, the therapeutic composition is delivered to the subject via one or more routes of administration. The multiple administrations may be rendered simultaneously or may be administered over a period of several hours or days. Additional therapy may be administered on a period basis, for example, daily, weekly or monthly.

Techniques for formulation and administration of the therapeutic compositions of the instant application may be found in “Remington's Pharmaceutical Sciences,” Mack Publishing Co., Easton, Pa., latest edition. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

### Dosing

The formulation of therapeutic compositions and their subsequent administration (dosing) is believed to be within the skill of those in the art, and determined, e.g., by dose-response, toxicity, and pharmacokinetic studies. Dosing is dependent on severity and responsiveness of the disease state to be treated, with the course of treatment lasting from several days to several months, or until a cure is effected or a diminution of the disease state is achieved.

### Diagnostic and Prognostic Methods

The invention furthermore provides diagnostic and prognostic methods relating to SCN5A splicing factors and splice variants, among others. The invention provides, for example, methods of identifying a subject at risk (e.g., at an increased risk) for arrhythmia or heart failure. In exemplary embodiments, the method comprises the step of determining a level of splicing factor hLuc7a, splicing factor RBM25 and/or PERK in a biological sample from the subject, wherein an increased level of hLuc7a, RBM25 and/or PERK in the sample compared to a control sample (e.g., a comparable sample from an individual known not to be at risk for arrhythmia or heart failure) indicates a risk (e.g., an increased risk) for arrhythmia or heart failure.

Arrhythmia

In exemplary embodiments, the invention provides a method of identifying a subject at risk for arrhythmia. As used herein, the term “arrhythmia” is synonymous with “cardiac dysrhythmia” or “cardiac arrhythmia” and refers to any condition in which there is abnormal electrical activity in the heart. In exemplary embodiments, the cardiac arrhythmia is a ventricular arrhythmia, such as ventricular fibrillation, ventricular tachycardia, or an arrhythmic condition in which both ventricular fibrillation and ventricular tachycardia are present. In exemplary embodiments, the cardiac arrhythmia is an atrial arrhythmia, e.g., an atrial fibrillation, atrial tachycardia, or an arrhythmic condition in which both atrial fibrillation and atrial tachycardia are present. Other types of cardiac arrhythmias are described below.

In exemplary aspects, the cardiac arrhythmia is characterized by an abnormal heart rate. In exemplary aspects, the cardiac arrhythmia is characterized by a bradycardia or a tachycardia.

Bradycardia

In exemplary aspects, the cardiac arrhythmia is a bradycardia in which the resting heart rate is slower than normal. In exemplary aspects, the bradycardia is characterized by a resting heart rate in an adult human which is slower than 60 beats per minute. In exemplary aspects, the bradycardia is a sinus bradycardia. In exemplary aspects, the bradycardia is caused by sinus arrest or AV block or heart block. In exemplary aspects, the bradycardia is caused by a slowed electrical conduction in the heart. In exemplary aspects, the bradycardia is not the bradycardia which is exhibited by the normally functioning heart of an athlete or athletic person.

Tachycardia

In exemplary aspects, the cardiac arrhythmia is a tachycardia in which the resting heart rate is faster than normal. In exemplary aspects, the tachycardia is characterized by a resting heart rate in an adult human which is faster than 100 beats per minute. In exemplary aspects, the tachycardia is a sinus tachycardia. In exemplary aspects, the sinus tachycardia is not caused by physical exercise, emotional stress, hyperthyroidism, ingestion or injection of substances, such as caffeine or amphetamines. In exemplary aspects, the tachycardia is not a sinus tachycardia, e.g., a tachycardia resulting from automaticity, reentry (e.g., fibrillation), or triggered activity. In exemplary aspects, the tachycardia is caused by a slowed electrical conduction in the heart. In exemplary aspects, the tachycardia is caused by an ectopic focus. In exemplary aspects, the tachycardia is combined with abnormal rhythm.

In exemplary aspects, the cardiac arrhythmia is characterized by the mechanism by which it occurs. In exemplary aspects, the cardiac arrhythmia is caused by automaticity, re-entry, or fibrillation.

Automaticity

In exemplary aspects, the cardiac arrhythmia is an abnormal rhythm or a tachycardia caused by automaticity, a condition in which a cardiac muscle cell other than a cardiac muscle of the conduction system fires an impulse of its own. In exemplary aspects, the cardiac arrhythmia is caused by a muscle cell, other than a cell of the sino-atrial (SA) node, atrial-ventricular (AV) node, Bundle of His, or Purkinje fibers, firing an impulse of its own.

Re-Entry

In exemplary aspects, the cardiac arrhythmia is a re-entry arrhythmia in which an electrical impulse recurrently travels in a circle within the heart, rather than moving from one end of the heart to the other and then stopping. In exemplary aspects, the cardiac arrhythmia is a cardiac flutter, a paroxysmal supraventricular tachycardia, or a ventricular tachycardia.

Fibrillation

In exemplary aspects, the cardiac arrhythmia is a fibrillation. In exemplary aspects, the fibrillation is an atrial fibrillation. In exemplary aspects, the fibrillation is a ventricular fibrillation.

Triggered Beats

In exemplary aspects, the cardiac arrhythmia is a triggered beat that occurs when ion channels in the heart cells malfunction, resulting in abnormal propagation of electrical activity and possibly leading to abnormal rhythm.

In exemplary embodiments, the cardiac arrhythmia is classified by site of origin. In exemplary aspects, the cardiac arrhythmia is an atrial arrhythmia (e.g., premature atrial contraction, wandering atrial pacemaker, multifocal atrial tachycardia, atrial flutter, atrial fibrillation). In exemplary aspects, the cardiac arrhythmia is a junction arrhythmia (e.g., supraventricular tachycardia, AV nodal reentral tachycardia, paroxysmal supraventricular tachycardia, junctional rhythm, junctional tachycardia, premature junctional complex). In exemplary aspects, the cardiac arrhythmia is an atrio-ventricular arrhythmia (e.g., AV reentrant tachycardia). In exemplary aspects, the cardiac arrhythmia is a ventricular arrhythmia (e.g., premature ventricular contraction or ventricular extra beat, accelerated idoventricular rhythm, monomorphic ventricular tachycardia, polymorphic ventricular tachycardia, ventricular fibrillation). In exemplary aspects, the cardiac arrhythmia is a heart block (e.g., first degree heart block, Type I second degree heart block, Type 2 second degree heart block, third degree heat block). In exemplary aspects, the cardiac arrhythmia is a premature contraction.

In exemplary aspects, the cardiac arrhythmia is a condition in which two or more types of cardiac arrhythmias are present. In exemplary aspects, the cardiac arrhythmia is a condition in which both ventricular tachycardia and ventricular fibrillation are present. In exemplary aspects, the cardiac arrhythmia is a condition in which a bradycardia is not present.

Heart Failure

In exemplary embodiments, the invention provides a method of identifying a subject at risk for heart failure. Heart failure (HF) is defined as the ability of the heart to supply sufficient blood flow to meet the body's needs. In some embodiments, the signs and symptoms of heart failure include dyspnea (e.g., orthopnea, paroxysmal nocturnal dyspnea), coughing, cardiac asthma, wheezing, dizziness, confusion, cool extremities at rest, chronic venous congestion, ankle swelling, peripheral edema or anasarca, nocturia, ascites, heptomegaly, jaundice, coagulopathy, fatigue, exercise intolerance, jugular venous distension, pulmonary rales, peripheral edema, pulmonary vascular redistribution, interstitial edema, pleural effusions, or a combination thereof. In some embodiments, the symptom of heart failure is one of the symptoms listed in the following table, which provides a basis for classification of heart failure according to the New York Heart Association (NYHA).

| NYHA | |
|---|---|
| Class | Symptoms |
| I | No symptoms and no limitation in ordinary physical activity, |
| | e.g. shortness of breath when walking, climbing stairs etc. |
| II | Mild symptoms (mild shortness of breath and/or angina) |
| | and slight limitation during ordinary activity. |
| III | Marked limitation in activity due to symptoms, even during less- |
| | than-ordinary activity, e.g.walking short distances (20-100 m). |
| | Comfortable only at rest. |
| IV | Severe limitations. Experiences symptoms even while at rest. |
| | Mostly bedbound patients. |

In exemplary aspects, the heart failure is a systolic heart failure, which is heart failure caused or characterized by a systolic dysfunction. In simple terms, systolic dysfunction is a condition in which the pump function or contraction of the heart (i.e., systole), fails. Systolic dysfunction may be characterized by a decreased or reduced ejection fraction, e.g., an ejection fraction which is less than 45%, and an increased ventricular end-diastolic pressure and volume. In some aspects, the strength of ventricular contraction is weakened and insufficient for creating an appropriate stroke volume, resulting in less cardiac output. In some aspects, the systolic heart failure is an ischemic heart failure. In alternative aspects, the systolic heart failure is a nonischemic heart failure.

Determining Step

In exemplary embodiments, the method comprises the step of determining a level of splicing factor hLuc7a, splicing factor RBM25 and/or PERK in a biological sample from the subject. In exemplary aspects, the level is an expression level of splicing factor hLuc7a, splicing factor RBM25 and/or PERK. In exemplary aspects, the expression level is a protein expression level, a gene expression level, or an mRNA expression level. Suitable methods of determining expression levels of proteins (e.g., hLuc7A, RBM25, PERK) are known in the art and include immunoassays (e.g., Western blotting, an enzyme-linked immunosorbent assay (ELISA), a radioimmunoassay (RIA), and immunohistochemical assay. Suitable methods of determining expression levels of nucleic acids (e.g., nucleic acids encoding hLuc7A, RBM25, PERK) are known in the art and include quantitative polymerase chain reaction (PCR), Northern blotting, and Southern blotting.

In exemplary embodiments, the method comprises the step of determining a level of splicing factor hLuc7a, splicing factor RBM25 and/or PERK in a biological sample from the subject, and the level that is determined is a biological activity level of splicing factor hLuc7a, splicing factor RBM25 and/or PERK, e.g., an enzymatic activity, a binding activity. In exemplary aspects, the biological activity level is an enzymatic activity level. In specific aspects, the enzymatic activity is reflected by the levels of the substrate or product of the enzymatic reaction catalyzed by splicing factor hLuc7a, splicing factor RBM25, or PERK. For example, the method may comprise determining a level of the phosphorylated product that PERK phosphorylates. In this regard, the method may comprise determining a level of phosphorylated PERK or phosphorylated eIF2.

Also, for example, the method may comprise determining a level of the splice variants produced by hLuc7A or RBM25, e.g., SCN5A splice variants, or a level of the precursor to the splice variants. The method of identifying a subject at risk for arrhythmias or heart failure may comprise in exemplary embodiments the step of determining a level of a full length transcript of SCN5A gene or of a splice variant of the SCN5A gene. Splice variants of the SCN5A gene are further described herein and in the art. See, e.g., U.S. Application Publication No. 2007/0212723 A1. In exemplary aspects, the method comprises determining a level of a full length transcript of SCN5A gene, and a decreased level of the full length transcript of the SCN5A gene indicates an increased risk for arrhythmia or heart failure. In exemplary aspects, the method comprises determining a level of a splice variant of the SCN5A gene, and an increased level of the splice variant indicates an increased risk for arrhythmia or heart failure. In specific aspects, the splice variant of the SCN5A gene is a splice variant produced from alternative splicing within Exon 28 of the SCN5A gene. In specific aspects, the splice variant is a SCN5A Exon 28 B splice variant (a.k.a., E28B), a SCN5A Exon 28 C splice variant (a.k.a., E28C), or a SCN5A Exon 28 D splice variant (a.k.a., E28D). Such splice variants of the SCN5A gene are further described herein.

In exemplary aspects, the method comprises screening for the presence of an abnormal SCN5A splice variant in a biological sample of the subject, wherein the presence of the abnormal splice variant identifies the subject as being at risk for developing arrhythmia. For example, the presence of one or more SCNA splice variants E28B (SEQ ID NO: 7), E28C (SEQ ID NO: 8) and/or E28D (SEQ ID NO: 9) in the biological sample identifies the subject as being at risk for developing arrhythmia. Thus the screening step, in some embodiments, comprises obtaining a biological sample from the subject and analyzing nucleic acid from the sample for the presence of an abnormal splice variant.

In exemplary aspects, the biological activity level is a binding activity level. In specific aspects, the binding activity level is reflected by the levels of the binding complex comprising hLuc7a, RBM25, or PERK and its binding partner. Methods of detecting binding complexes include, for example, immunoprecipitation using an antibody specific for hLuc7a, RBM25, or PERK followed by Western blotting with an antibody specific for its binding partner.

In exemplary embodiments, the method comprises the step of determining a level of splicing factor hLuc7a, splicing factor RBM25 and/or PERK in a biological sample from the subject, and the level that is determined is represented by the level of biological activity of a related protein, e.g., a protein which acts upstream or downstream of the hLuc7A, RBM25, PERK. For example, the method may comprise determining a level of a chaperone protein (e.g., calnexin, CHOP), since these proteins are upregulated once the UPR is activated via PERK.

Accordingly, the method of identifying a subject at risk for arrhythmias or heart failure, in additional or alternative embodiments, comprises the step of determining a level of a chaperone protein in a biological sample from the subject, wherein an increased level of the chaperone protein in the sample compared to a control sample indicates an increased risk for arrhythmia or heart failure. In exemplary embodiments, the chaperone protein is CHOP or calnexin.

In exemplary aspects, the chaperone protein is CHOP. CHOP (NCBI Gene ID No. 1649) is also known as DDIT3, DNA-damage-inducible transcript 3, CEBPZ; CHOP10; CHOP-10; GADD153; MGC4154. Exemplary amino acid sequences of CHOP are set forth herein as SEQ ID NOs: 42-47 but are also found in the NCBI's Protein database as Accession Nos. NP_{—}001181982.1, NP_{—}001181983.1, NP_{—}001181984.1, NP_{—}001181985.1, NP_{—}001181986.1, NP_{—}004074.2. Exemplary nucleotide sequences of CHOP are set forth herein as SEQ ID NO: 48-53 but are also found in the NCBI's Nucleotide database as Accession Nos. NM_{—}001195053.1, NM_{—}001195054.1, NM_{—}001195055.1, NM_{—}001195056.1, NM_{—}001195057.1, and NM_{—}004083.5

In exemplary aspects, the chaperone protein is calnexin. Calnexin (NCBI Gene ID No. 821) is also known as CANX; CNX; P90; IP90; FLJ26570. Exemplary amino acid sequences of calnexin are set forth herein as SEQ ID NOs: 54 and 55 but are also found in the NCBI's Protein database as Accession Nos. NP_{—}001019820.1 and NP_{—}001737.1. Exemplary nucleotide sequences of Calnexin are set forth herein as SEQ ID NO: 56 and 57 but are also found in the NCBI's Nucleotide database as Accession Nos. NM_{—}001024649.1 and NM_{—}001746.3.

In the methods in which the level of a chaperone protein is determined, the level may be an expression level (e.g., a protein level, an mRNA level, gene expression level) of the chaperone protein. Alternatively, the level may be a biological activity level, such as, an enzymatic activity level or a binding activity level. Alternatively, the level may be a level of biological activity of a related protein. Suitable methods of determining such levels are further described herein.

Sudden Cardiac Death

Arrhythmias and heart failure are related to sudden cardiac death (SCD). For example, SCD is responsible for about 50% of deaths from heart failure and often is the first expression of coronary disease. See, Sovari et al., “Sudden Cardiac Death,” e-medicine Cardiology, article 151907, updated Nov. 4, 2010; and Zheng et al., Circulation 104: 2158-2163 (2001). A common cause of SCD is ventricular arrhythmia, including, for example, ventricular tachycardia (VT), in which the resting heart rate is faster than normal, ventricular fibrillation (VF), in which there is uncoordinated contraction of the cardiac muscle of the ventricles in the heart, making the muscles quiver rather than contract properly, or an arrhythmic condition in which both VT and VF are present. See, Wedro, B., “Sudden Cardiac Arrest (Sudden Cardiac Death),” medicine.net, Kulick and Soppler, eds.

Accordingly, the invention also provides methods of identifying a subject at risk (e.g., at increased risk) for sudden cardiac death. In exemplary embodiments, the method comprises the step of determining a level of splicing factor hLuc7a, splicing factor RBM25 and/or PERK in a biological sample from the subject, wherein an increased level of hLuc7a, RBM25 and/or PERK in the sample compared to a control sample (e.g., a comparable sample from an individual known not to be at risk for SCD) indicates a risk (e.g., increased risk) for SCD.

The level that is determined may be an expression level (e.g., a protein level, an mRNA level, gene expression level) of hLuc7A, RBM25, and/or PERK. Alternatively, the level may be a biological activity level, such as, an enzymatic activity level or a binding activity level. Alternatively, the level may be a level of biological activity of a related protein. Suitable methods of determining such levels are further described herein.

Hypertrophic Cardiomyopathy

Hypertrophic cardiomyopathy (HCM) is a condition in which the heart muscle becomes thick, and the thickened heart tissue makes it harder for blood to leave the heart, forcing the heart to work harder to pump blood. HCM is often asymmetrical—one or more parts of the heart is thicker than the others. The condition is usually inherited, wherein genes that control heart muscle growth are defective. While younger people are likely to have a more severe form of HCM, the condition is seen in people of all ages.

The invention moreover provides a method of diagnosing a subject with hypertrophic cardiomyopathy (HCM) or determining a subject's risk for developing HCM. In exemplary embodiments, the method comprises the step of determining a level of E28D, RBM25, PRPF40A, or LUC7L3 (a.k.a., hLuc7A), or a combination thereof, in a sample obtained from the subject, wherein an increased level is indicative of the subject having HCM or an increased risk of developing HCM.

HCM can lead to further medical complications (e.g., medical complications associated with HCM), some of which are life-threatening. The invention also provides a method of prognosticating a subject with HCM. In exemplary embodiments, the method of prognosticating the HCM subject is a method of determining the HCM subject's risk for sudden cardiac death, arrhythmias (e.g., atrial arrhythmias), heart failure, and/or dilated cardiomyopathy. In exemplary embodiments, the method comprises the step of determining a level of E28D, RBM25, PRPF40A, or LUC7L3, or a combination thereof, in a sample obtained from the subject, wherein a modified level, as compared to a control sample, is indicative of a poor prognosis, need for treatment, and/or an increased risk for sudden cardiac death, arrhythmias (e.g., atrial arrhythmias), heart failure, and/or dilated cardiomyopathy.

Accordingly, the invention provides a method of determining risk of sudden cardiac death in a subject with HCM, comprising the step of determining a level of E28D, RBM25, PRPF40A, or LUC7L3, or a combination thereof, in a sample obtained from the subject, wherein a modified expression level, as compared to a control sample, is indicative of an increased risk of sudden cardiac death.

The invention also provides a method of determining risk of arrhythmias (e.g., atrial arrhythmias) in a subject with HCM, comprising the step of determining a level of E28D, RBM25, PRPF40A, or LUC7L3, or a combination thereof, in a sample obtained from the subject, wherein a modified expression level, as compared to a control sample, is indicative of an increased risk of arrhythmias.

Further provided is a method of determining a risk of heart failure in a subject with HCM, comprising the step of determining a level of E28D, RBM25, PRPF40A, or LUC7L3, or a combination thereof, in a sample obtained from the subject, wherein a modified expression level, as compared to a control sample, is indicative of an increased risk of heart failure.

The invention furthermore provides a method of determining a risk of dilated cardiomyopathy in a subject with HCM, comprising the step of determining a level of E28D, RBM25, PRPF40A, or LUC7L3, or a combination thereof, in a sample obtained from the subject, wherein a modified expression level, as compared to a control sample, is indicative of an increased risk of dilated cardiomyopathy.

The level that is determined in a sample obtained from the HCM subject may be an expression level (e.g., a protein level, an mRNA level, gene expression level) of LUC7L3, RBM25, and/or PRPF40A. Alternatively, the level may be a biological activity level, such as, an enzymatic activity level or a binding activity level. Alternatively, the level may be a level of biological activity of a related protein. Suitable methods of determining such levels are further described herein.

In exemplary aspects, the level that is determined is a level of PRPF40A. PRPF40A (NCBI Gene ID No. 55660) is also known as PRP40 pre-mRNA processing factor 40 homolog A, HYPA; FBP11; FLAF1; FNBP3; HIP10; Prp40; FBP-11; HIP-10; FLJ20585; and NY-REN-6. An exemplary amino acid sequence of PRPF40A is set forth herein as SEQ ID NO: 58 but is also found in the NCBI's Protein database as Accession No. NP_{—}060362.3. An exemplary nucleotide sequence of PRPF40A is set forth herein as SEQ ID NO: 59 but is also found in the NCBI's Nucleotide database as Accession No. NM_{—}017892.3.

In exemplary embodiments, the modified expression level that is determined is an increased level, as compared to a control sample, and the increased level is indicative of an increased risk of sudden cardiac death, arrhythmias (e.g., atrial arrhythmias), heart failure, or dilated cardiomyopathy.

Samples

With regard to the methods disclosed herein, in some embodiments, the sample comprises a bodily fluid, including, but not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of the foregoing samples. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukopheresis). In exemplary aspects, the sample comprises white blood cells obtained from the subject. In exemplary aspects, the sample comprises only white blood cells. In exemplary aspects, the sample is muscle tissue (e.g., skeletal muscle tissue). In exemplary aspects, the sample is cardiac tissue (e.g., cardiac muscle tissue).

Subjects

With regard to the methods disclosed herein, the subject in exemplary aspects is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human.

Control Levels

In the diagnostic and prognostic methods described herein, the level that is determined may be the same as a control level, or may be increased or decreased relative to a control level. In some embodiments, the control level is the level of the marker (e.g., hLuc7A, RBM25, PERK, E28D, PRPF40A, etc) determined in a sample obtained from a control subject, wherein the control subject is known to not have the disease, or a risk thereof, (e.g., arrhythmias, heart failure, sudden cardiac death, HCM, sudden cardiac death, arrhythmias (e.g., atrial arrhythmias), heart failure, and/or dilated cardiomyopathy). In some aspects, the control subject is a matched control of the same species, gender, ethnicity, age group, smoking status, BMI, current therapeutic regimen status, medical history, or a combination thereof, but differs from the subject being diagnosed in that the control does not suffer from the disease in question.

Relative to a control level, the level that is determined may an increased level. As used herein, the term “increased” with respect to level (e.g., expression level, biological activity level) refers to any % increase above a control level. The increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

Relative to a control level, the level that is determined may a decreased level. As used herein, the term “decreased” with respect to level (e.g., expression level, biological activity level) refers to any % decrease below a control level. The decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

Methods for Determining Need for Therapy or Prophylaxis

Since accurate diagnosis of a subject leads to determining the appropriate therapy for treating the diagnosed medical condition, disease, or syndrome, the invention also provides related methods of determining need for therapy or prophylaxis of a subject. For example, the invention provides methods of determining need for therapy or prophylaxis for arrhythmia or heart failure of a subject identified as having a risk (e.g., an increased risk) for arrhythmia or heart failure. The method comprises the step of determining a level of splicing factor hLuc7a, splicing factor RBM25 and/or PERK in a biological sample from the subject, wherein an increased level indicates the need for a therapy or prophylaxis for arrhythmia or heart failure. The invention also provides methods of determining need for therapy or prophylaxis for SCD of a subject identified as having a risk (e.g., an increased risk) for SCD. The method comprises the step of determining a level of splicing factor hLuc7a, splicing factor RBM25 and/or PERK in a biological sample from the subject, wherein an increased level indicates the need for a therapy or prophylaxis for SCD. The invention additionally provides a method of determining need for therapy for HCM. The method comprises the step of determining a level of E28D, RBM25, PRPF40A, or LUC7L3 (a.k.a., hLuc7A), or a combination thereof, in a sample obtained from the subject, wherein an increased level is indicative of the subject needing therapy for HCM. Further, the invention provides methods of determining need for therapy or prophylaxis of a medical complication associated with HCM. The method comprises the step of determining a level of E28D, RBM25, PRPF40A, or LUC7L3 (a.k.a., hLuc7A), or a combination thereof, in a sample obtained from the subject, wherein an increased level is indicative of the subject needing therapy or prophylaxis of the medical complication. In exemplary aspects, the medical complication associated with HCM is sudden cardiac death, arrhythmias (e.g., atrial arrhythmias), heart failure, or dilated cardiomyopathy.

Methods for Decreasing Risk

Since accurate diagnosis of a subject of having an increased risk for a medical condition, disease, or syndrome, intervention via active therapy or active prophylaxis may decrease the risk for developing the medical condition, disease, or syndrome. Accordingly, the invention also provides related methods of decreasing risk of arrhythmias of a subject. The method comprises the steps of (i) determining a level of splicing factor hLuc7a, splicing factor RBM25 and/or PERK in a biological sample from the subject, and (ii) administering to the subject an anti-arrhythmias therapeutic or prophylactic agent, if the level determined in (i) is increased. The invention likewise provides methods of decreasing risk of heart failure of a subject. The method comprises the steps of (i) determining a level of splicing factor hLuc7a, splicing factor RBM25 and/or PERK in a biological sample from the subject, and (ii) administering to the subject an anti-heart failure therapeutic or prophylactic agent, if the level determined in (i) is increased. The invention further provides methods of decreasing risk of SCD of a subject. The method comprises the steps of (i) determining a level of splicing factor hLuc7a, splicing factor RBM25 and/or PERK in a biological sample from the subject, and (ii) administering to the subject an anti-SCD therapeutic or prophylactic agent, if the level determined in (i) is increased. The invention furthermore provides methods of decreasing risk of developing a medical complication associated with HCM. The method comprises the step of (i) determining a level of E28D, RBM25, PRPF40A, or LUC7L3 (a.k.a., hLuc7A), or a combination thereof, in a sample obtained from the subject, and (ii) administering to the subject a therapeutic or prophylactic agent for the medical complication, if the level determined in (i) is increased.

Methods of Monitoring Risk

The invention additionally provides methods of monitoring risk of a medical condition, disease, or syndrome. For example, the invention provides methods of monitoring risk of arrhythmias of a subject. The method comprises the step determining a level of splicing factor hLuc7a, splicing factor RBM25 and/or PERK in a biological sample from the subject, wherein a decreased level, relative to a control level, is indicative of a decreased risk or arrhythmias. The invention also provides methods of monitoring risk of heart failure of a subject. The method comprises the step determining a level of splicing factor hLuc7a, splicing factor RBM25 and/or PERK in a biological sample from the subject, wherein a decreased level, relative to a control level, is indicative of a decreased risk for heart failure. The invention also provides methods of monitoring risk of SCD of a subject. The method comprises the step determining a level of splicing factor hLuc7a, splicing factor RBM25 and/or PERK in a biological sample from the subject, wherein a decreased level, relative to a control level, is indicative of a decreased risk for SCD. The invention furthermore provides methods of monitoring risk of developing a medical complication associated with HCM. The method comprises the step of determining a level of E28D, RBM25, PRPF40A, or LUC7L3 (a.k.a., hLuc7A), or a combination thereof, in a sample obtained from the subject, wherein a decreased level, relative to a control level, is indicative of a decreased risk for the medical complication.

The invention further provides a method of monitoring progression of a disease, e.g., HCM, in a subject. The method comprises the step of determining a level of E28D, RBM25, PRPF40A, or LUC7L3 (a.k.a., hLuc7A), or a combination thereof, in a sample obtained from the subject, wherein a decreased level, relative to a control level, is indicative disease regression.

The control level in these methods of monitoring risk may be, for example, a level taken at a time prior to intervention (e.g., prior to administration of a therapeutic or prophylactic agent), or simply a prior time (e.g., if monitoring disease progression and no treatment or prophylaxis is administered to the subject).

Treatment and Prevention

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of diastolic dysfunction or heart failure in a mammal. Furthermore, the treatment or prevention provided by the inventive method can include treatment or prevention of one or more conditions or symptoms of the disease being treated or prevented. Also, for purposes herein, “prevention” can encompass delaying the onset of the disease, or a symptom or condition thereof.

Kits

Provided herein are kits, e.g., diagnostic kits, that may be used to diagnose or determine risk, in accordance with the methods set forth herein. In exemplary embodiments, the kit comprises two or more of: (a) an hLuc7A binding agent, (b) an RBM25 binding agent, (c) a PERK binding agent, (d) a PRPF40A binding agent, (e) a SCN5A splice variant E28D, and instructions for use.

In exemplary embodiments, the kit is a diagnostic kit for determining risk of arrhythmias, heart failure or SCD, and the kit comprises a hLuc7a binding agent, a RBM25 binding agent, and/or a PERK binding agent. The kits in specific aspects may additionally comprise binding agents for chaperone proteins, e.g., CHOP and calnexin, and/or binding agents for SCN5A splice variants or binding agents for full-length SCN5A transripts.

In exemplary embodiments, the kit is a diagnostic kit for diagnosing HCM or a kit for determining risk for a medical complication associated with HCM, and the kit comprises an E28D binding agent, a hLuc7a binding agent, a RBM25 binding agent, and/or a PRPF40A binding agent.

As used herein, the term “binding agent” refers to any compound which specifically binds to the marker of interest (hLuc7A, RBM25, PERK, E28D, PRPF40A, and the like). In some aspects, the binding agent is an antibody, antigen binding fragment, an aptamer, a protein or peptide substrate, or a nucleic acid probe. Such binding agents are known in the art. In exemplary aspects, the kit comprises one or more antibodies or antigen binding fragments thereof that specifically bind to hLuc7A, RBM25, PERK, PRPF40A, or E28D, and/or, the kit comprises one or more nucleic acid probes that hybridize to a nucleic acid encoding hLuc7A, RBM25, PERK, PRPF40A, or E28D. In some aspects, the kit comprises a collection of nucleic acid probes which specifically bind to genes or nucleic acids encoding the marker. In some aspects, the collection of nucleic acid probes is formatted in an array on a solid support, e.g., a gene chip. In some aspects, the kit comprises a collection of antibodies which specifically bind to a marker. In some aspects, the kit comprises a multi-well microtiter plate, wherein each well comprises an antibody having a specificity which is unique to the antibodies of the other wells. In some aspects, the kit comprises a collection of substrates which specifically react with a marker. In some aspects, the kit comprises a multi-well microtiter plate, wherein each well comprises a substrate having a specificity which is unique to the substrates of the other wells.

In some aspects, the kits further comprises instructions for use. In some aspects, the instructions are provided as a paper copy of instructions, an electronic copy of instructions, e.g., a compact disc, a flash drive, or other electronic medium. In some aspects, the instructions are provided by way of providing directions to an internet site at which the instructions may be accessed by the user. In exemplary aspects, the instructions comprise instructions for determining an expression level of hLuc7A, RBM25, PERK, PRPF40A, or E28D in a biological sample.

In some aspects, the instructions comprise a step in which the user compares data relating to a marker to a database containing correlation data. In some aspects, the kit comprises an electronic copy of a computer software program which allows the user to compare the determined level of the marker with that of a control subject.

In alternative aspects, the instructions comprise a step in which the user provides data relating to the level of the marker to a provider and the provider, after analyzing the data, provides diagnostic information to the user.

In some aspects, the kits further comprise a unit for a collecting a sample, e.g., any of the samples described herein, of the subject. In some aspects, the unit for collecting a sample is a vial, a beaker, a tube, a microtiter plate, a petri dish, and the like.

The following examples serve only to illustrate the invention and are not intended to limit the scope of the invention in any way.

### EXAMPLES

The following materials and methods were used in at least Examples 1-3.

Microarray data analysis: Changes in splicing factor mRNA abundances were evaluated by microarray analysis comparing human HF to normal myocardium. The data was uploaded to GeneSifter using Batch Upload with the option to use Affymetrix probe IDs. The data was log 2 transformed and quantile normalized. Statistically significant genes were identified by using an unpaired Student t test (p value <0.05 and a 5% Benjamini and Hochberg false discovery rate correction). A range of fold change cut offs were used. Genes associated with RNA splicing were found under the biological process GO term “GO: 0008380: RNA splicing”. The significance of the observed number of genes associated with RNA splicing was determined using z scores.

Cell culture assays: Jurkat T cell clones E6.1 (ATCC, Manassas, Va.) were cultured at 37° C. and 5% CO2 in RPMI 1640 medium supplemented with 10% heat inactivated fetal calf serum, 4 mM glutamine, 75 units/mL streptomycin and 100 units/mL penicillin. WA09 (H9) human undifferentiated ESCs were obtained through the National Stem Cell Bank and maintained on irradiated mouse embryonic fibroblasts at 37° C. with 5% CO2. The culture medium consisted of DMEM/F12 (Invitrogen, Carlsbad, Calif.) supplemented with 15% knockout serum, 1% non-essential amino acids, 1 mmol/L L-glutamine, 0.1 mmol/L β-mercaptoethanol, and basic fibroblast growth factor of 20 ng/mL.

Real-Time PCR quantification: Total RNA was isolated from cultured cells using the Qiagen RNeasy Mini Kit (Valencia, Calif.). Total RNA from human ventricles was isolated using the RNeasy Lipid Tissue Mini Kit (Qiagen). Reverse transcription was carried out at 42° C. for 1 h with Powerscript reverse transcriptase (Roche). β-Actin was used as a reference when making quantitative comparison. The primers for target genes are listed in Table 1.

TABLE 1
PRIMERS FOR TARGET GENES.

| | | SEQ |
|---|---|---|
| Gene | Primer | ID NO |
| SCN5A | 5′-TTACGCACCTTCCGAGTCCTCC-3′ | 18 |
| | 5′-GATGAGGGCAAAGACGCTGAGG- 3′ | 19 |
| HSCN5AE28C/R | 5′-TCTCTTCTCCCCTCCTGCTGGTCA-3′ | 20 |
| HSCN5AE28D/R | 5′-GGAAGAGCGTCGGGGAGAAGAAGTA-3′ | 21 |
| PERK | 5′-AGTCTCTGCTGGAGTCTTCA-3; | 22 |
| | 5′-TGACACTGTGTCTCAGACTCTT-3′ | 23 |
| RBM25 | 5′-TGTCTTTTCCACCTCATTTGAATCG-3; | 24 |
| | 5′-ATTGGTACAGGAATCATTGGGGT-3′ | 25 |
| hLuc7a | 5′-GGACCAAGATCAGAACGTGTATTTG-3; | 26 |
| | 5′-CAGTTGTTGGATGAGTTAATGGGC-3′ | 27 |
| sXBP1 | 5′-TCTGCTGAGTCCGCAGCAGG-3′; | 28 |
| | 5′-CTCTAAGACTAGAGGCTTGG-3 ′ | 29 |
| β-actin | 5′-GGATCGGCGGCTCCAT-3; | 30 |
| | 5′-CATACTCCTGCTTGCTGATCCA-3′ | 31 |

Transfection Assays: Fugene 6 reagents (Roche, Indianapolis, Ind.) were used for transfection assays. Transfection methods followed the manufacturer's instructions. siRNA for hLuc7A and RBM25 were purchased from Invitrogen. The expression plasmid pcDNA3.1-HA-RBM25 was kindly provided by Dr. Huang (Dana-Farber Cancer Institute, Harvard, Boston, Mass.). Details of the E28C and E28D expression vectors have been discussed previously.

Gel mobility shift assays: The biotinylated wild-type (CAGCAGGCGGGCAGCGGCCU) and mutant (CAGCAGGUUAGAGGCGGCCU) RNA substrates (SEQ ID NOs: 32 and 33, respectively) were synthesized by Invitrogen. Binding of biotinylated RNA to RBM25 was achieved by incubating 0.2 nM RNA and variable amounts of protein for 30 min at 4° C. in 20 μL of binding buffer (10 mM Tris-HCl, 10 mM HEPES, 100 mM NaCl, 0.1% Triton X-100, 2 mM MgCl₂, 1.5 mM dithiothreitol (DTT) pH 7.5, 7% glycerol). For the competition assays, a molar excess of unlabeled competitor RNAs at various fold levels was added to the pre-incubated reaction mixture. Samples were fractionated in a native 5% polyacrylamide gel, transferred to Hybond-N+nylon membrane (Amersham Biosciences), and detected with a LightShift chemiluminescent electrophoretic mobility shift assay kit (Pierce) by following the manufacturer's protocol.

Western blot analysis: The Mini-PROTEAN® Tetra Electrophoresis System from BioRad was used for Western blot analysis. Anti-Nav1.5 antibody, recognizing all channel variants, was provided by Dr. Peter Mohler (University of Iowa, Iowa City, Iowa). Anti-RBM25 antibody was provided by Dr. Huang (Dana-Farber Cancer Institute, Harvard, Boston, Mass.). Anti-PERK and hLuc7A antibodies were purchased from R&D Systems (Minneapolis, Minn.) and Millipore, (Billerica, Mass.).

Statistical Evaluations: All data are presented as means±SEM. Means were compared by using Student t tests. A probability value of p<0.05 was considered statistically significant.

### Example 1

This example demonstrates that splicing factors hLuc7A and RBM 25 are associated with abnormal splicing of SCN5A.

To identify SCN5A candidate splicing factors, microarray analysis of human heart samples from patients with and without heart failure was undertaken to look at changes in mRNA splicing factor abundance. Heart samples from mice with (n=10) and without (n=10) heart failure served to help limit the candidate splicing factors, since mice do not show SCN5A variants in either condition.

The data were uploaded to GeneSifter using Batch Upload with the option to use Affymetrix probe IDs. The data were log 2 transformed and quantile normalized. Statistically significant genes were identified by using an unpaired Student t test (p value <0.05) and a 5% Benjamini and Hochberg false discovery rate (FDR) correction. A range of fold change cut offs was used. Genes associated with RNA splicing were found under the biological process GO term “GO: 0008380: RNA splicing”. The significance of the observed number of genes associated with RNA splicing was determined using z scores. When known, binding sequences of upregulated factors were compared with the genomic sequence of SCN5A.

From this analysis using a 1.2-fold cutoff, 47 splicing factors were identified as upregulated in HF (Table 2).

TABLE 2
A comprehensive list of splicing factors
altered in human heart failure

| | |
|---|---|
| Hypoxia related | SF4; CROP; SYF2; TARDBP; SF1; SFRS7; |
| genes | HNRPH3; PPIE; PRPF40A; PPIG; CDC5L; |
| | HNRNPC; PRPF31; RBM25. |
| Inflammation | C19orf29; SRRM1; FRG1; BAT1; RBM39; |
| related genes | PNN; IVNS1ABP; RBM8A. |
| Hormone level | SF4; BAT1; RBM8A; SNRPD3. |
| changing related | |
| genes | |
| Pressure overload | RBM39; SNRPD3. |
| related genes | |
| Others | ZNF638; PRP4; TSEN34; YTHDC1; |
| | RBM22; TFIP11; RNPS1; SFPQ; WBP4; |
| | DHX35; SFRS11; QKI; SFRS14; DGCR14; |
| | SFRS12; RY1; SNW1; NCBP2; SF3B1; |
| | WBP11; MPHOSPH10; HNRNPA1; TRA2A; |
| | SFRS5. |

Most of the 47 identified splicing factors are regulated by hypoxia or inflammation, both conditions usually present in heart failure. In heart failure samples, hLuc7a and RBM25 were upregulated by 1.7 and 1.5 fold, respectively. The upregulation of splicing factors RBM25 and hLuc7A in human heart failure tissue was confirmed by RT-PCR. Compared to the normal heart tissue, the results indicated that the relative abundances of RBM25 and hLuc7A were increased by 109.5±4.8% and 57.2±3.5% in heart failure tissue respectively (p<0.05). mRNA findings were correlated with protein expression by Western blot. Compared to the control group (mixture of 4 normal heart tissue samples), Western blot analysis showed that RBM25 was increased by 56.9±7.5%, 54.8±7.1%, 49.2±6.2% and 53.5±6.9% in heart failure tissue samples 1-4, respectively (p<0.05). hLuc7A was increased by 67.4±7.8%, 53.6±6.7%, 65.7±7.4% and 61.1±7.3% in the same heart failure samples (p<0.05).

As stated above, these hLuc7a and RBM25 act together to mediate splicing of SCN5A and RBM25 binds target RNA (Zhou et al., Mol. Cell. Biol., 28:5924-5936, 2008). There was only one canonical RBM25 site in SCN5A, and it was in exon 28 near the E28C and E28D splice sites. hLuc7a was absent from mouse heart. Therefore, splicing factors hLuc7a and RBM25 were upregulated in heart failure, recognized a sequence in SCN5A near the abnormal splicing, and a component was missing in mouse, potentially explaining why mice do not show abnormal SCN5A splicing in heart failure. Thus, hLuc7a and RBM25 became favored candidate factors mediating abnormal SCN5A splicing in HF.

Next, we showed that RBM25 bound to the canonical sequence, CGGGCA, in SCN5A exon 28. Gel mobility shift assays were performed as described previously with modification (Shang et al., Am. J. Physiol. Cell. Physiol., 292:C886-95, 2007, the disclosure of which is incorporated herein by reference in its entirety.). The biotinylated wild-type (CAGCAGGCGGGCAGCGGCCU) and mutant (CAGCAGGUUAGAGGCGGCCU) RNA substrates (SEQ ID NOs: 32 and 33, respectively) were synthesized. Binding of biotinylated RNA to RBM25 was achieved by incubating 0.2 nM RNA and variable amounts of protein for 30 min at 4° C. in 20 μL binding buffer. For the competition assays, a molar excess of unlabeled competitor RNAs at various fold levels was added to the pre-incubated reaction mixture. Results showed RBM25 binding with the CGGGCA sequence. Specificity was confirmed by showing a lack of this binding to a mutated canonical binding sequence. RBM25 bound the wild-type SCN5A sequence in a concentration-dependent manner. Specificity was inferred from the inability of RBM25 to bind the mutant sequence and for unlabeled probe to compete with labeled probe for RBM25 binding. In summary, splicing factors hLuc7A and RBM25 are upregulated in human heart failurs and bind a sequence in the SCN5A exon 28, where pathological splicing occurs.

### Example 2

This example demonstrates Ang-II and hypoxia regulate RBM25, hLuc7A, and SCN5A mRNA splicing.

Because AngII and hypoxia are common in heart failure, we investigated whether these two conditions could influence RBM25 and hLuc7A levels. Since only human white blood cells and cardiac cells express SCN5A and have demonstrated similar SCN5A mRNA splicing (Shang et al., Circ. Res., 101:1146-1154, 2007), Jurkat cells and H9 hESC-cardiomyocytes (CMs) were used for further testing. The Jurkat cells and H9 hESC-CMs were divided into three experiment groups: normoxia, hypoxia-treated (1% O₂), and Ang II-treated (100 nmol/L). The cells were harvested from each experiment group at four time points (30 min, 24 h, 48 h, and 72 h), and total mRNA extracted. Results indicated that mRNA abundances of both RBM25 and hLuc7A were increased in both cell types. Under hypoxia-treated condition, the expressions of RBM25 and hLuc7A in Jurkat cells were increased by 53.7±5.1% and 487.5±8.2%, respectively (p<0.05), and the expressions of RBM25 and hLuc7A in ES cells were increased by 57.9±5.2% and 389.5±7.9%, respectively (p<0.05). Under Ang II-treated condition, the expressions of RBM25 and hLuc7A in Jurkat cells were increased by 50.8±4.9% and 187.3±7.9%, respectively (p<0.05), and the expressions of RBM25 and hLuc7A in ES cells were increased by 42.1±4.7% and 181.2±6.7%, respectively (p<0.05).

The upregulation of splicing factors RBM25 and hLuc7A in Jurkat cells was further confirmed by Western blot. The Jurkat cells were divided into three experiment groups: normoxia, hypoxia-treated (1% O₂), and Ang II-treated (100 nmol/L). The expressions of RBM25 and hLuc7A were analyzed by Western blot at three time points (12 h, 24 h, and 48 h) for hypoxia-treated group and at four time points (24 h, 48 h, 72 h, and 96 h) for Ang II-treated group. Compared to the control group, Western blot analysis showed that the density of RBM25 was increased by 188.4±8.6%, 196.5±8.9% and 147.7±8.1% in hypoxia-treated group at time points 12 h, 24 h and 48 h, respectively, and was increased by 209.5±8.8%, 212.6±9.7%, 181.3±8.3% and 198.8±8.7% in the Ang II-treated group at time points 24 h, 48 h, 72 h and 96 h, respectively (p<0.05). The density of hLuc7A was increased by 242.3±9.5%, 236.1±8.4% and 268.8±9.8% in hypoxia-treated group at time points 12 h, 24 h and 48 h, respectively, and was increased by 256.3±9.1%, 246.5±8.6%, 279.7±9.3% and 283.6±9.9% in Ang II-treated group at time points 24 h, 48 h, 72 h and 96 h, respectively (p<0.05).

Changes in hLuc7A and RBM25 correlated with SCN5A E28C and E28D variant abundances. Under hypoxia, the expression of SCN5A variants E28C and E28D were increased by 373.5±5.7% and 636.2±7.6%, respectively (p<0.05), while the expressions of the full length SCN5A transcript was decreased by 64.6±4.9% (p<0.05). With Ang II treatment, the expressions of the variants were increased by 291.4±5.2% and 433.7±6.5% for E28C and E28D, respectively (p<0.05), while the expression of the full length SCN5A transcipt was decreased by 78.9±5.4% (p<0.05).

In order to show that the increase in RBM25 and hLuc7A mediated the abnormal SCN5A mRNA splicing, RBM25 and hLuc7A expression was suppressed with siRNA. siRNA for the two splicing factors were found to partially reverse the expressions of the induced SCN5A variants E28C and E28D at 48 h. The siRNA knockdown efficiency was estimated by both RT-PCR and Western blot and was not less than 50%. The expressions of SCN5A and the variants E28C and E28D were measured by RT-PCR in all experiment groups at time points of maximal siRNA effect.

### Example 3

This example demonstrates that SCN5A variants activate the unfolded protein response (UPR).

Previously, we have shown that SCN5A splicing variants have a dominant negative effect on Na⁺ current (Shang et al., Circ. Res., 101:1146-1154, 2007). Since the Na⁺ channel is encoded by a single mRNA, it is unclear how truncated forms might have a dominant negative effect on full-length channel production. The following Example investigated whether truncated SCN5A variant activate the unfolded protein response (UPR) pathway.

Hypoxia and AngII were used to increase abnormal SCN5A splicing. The expressions of PERK and sXBP1 were measured by RT-PCR. The expression of PERK was increased at 48 h by 18.6±0.8 fold (p<0.05) and 14.2±0.6 fold (p<0.05) under hypoxia-treated and Ang II-treated conditions respectively, while no expression upregulation of sXBP1 was observed. To test if this upregulation of one arm of the UPR was mediated by SCN5A mRNA variants, exogenous E28C and E28D were introduced. The Jurkat cells were divided into four experiment groups: normal control, empty vector control, variant E28C overexpressioned cells, and variant E28D overexpressioned cells. The expression of PERK was measures by RT-PCR in each group at the time point 48 h. Results indicated that the expression of PERK was increased by 6.3±0.4 (p<0.05) fold and 7.9±0.5 fold (p<0.05) when the variants E28C or E28D were overexpressed. The upregulation of PERK in Jurkat cells was further confirmed by Western blot. Compared to the control group, Western blot analysis showed that the density of PERK was increased by 437.9±11.2%, 383.2±10.7% under hypoxia-treated and Ang II-treated conditions respectively (p<0.05), and was increased by 262.6±9.6% and 359.5±10.1% in cells overexpressing variants E28C or E28D respectively (p<0.05). Furthermore, siRNA against PERK partially reversed the downregulation of full-length SCN5A expression after hypoxia or Ang II treatment. siRNA knockdown efficiency not less than 50%.

Discussion of Examples 1 to 3:

In order to explore the mechanism on downregulation of SCN5A full-length Na⁺ channel, the correlation of expression changes between UPR components and full-length Na⁺ channel as well as SCN5A variants is analyzed in the following. The UPR is a series of interrelated signaling pathways that occur when the ER experiences excess secretory load, accumulates misfolded proteins, or is subject to other pathological conditions. The UPR acts on several levels: it rapidly attenuates general protein synthesis, induces the expression of ER chaperone proteins, and enhances the degradation of misfolded proteins. These changes are presumably designed to restore protein folding and ER health. Nevertheless, prolonged activation of the UPR leads to apoptosis and has been implicated in the death of beta cells in type II diabetes mellitus. There appear to be three main sensor proteins that activate UPR. These transmembrane ER proteins are: protein kinase R-like ER kinase (PERK), inositol-requiring protein 1 (IRE1), and activating transcription factor 6 (ATF-6). It is reported that activated ATF-6 will in turn induce the downstream molecule sXBP1. While activated by endoribonuclease domain, IRE1 splices a 26 nucleotide fragment out of the XBP1 mRNA and generates sXBP1 (frame-shift splice variant of XBP1). Therefore, the increase of sXBP1 in cells can be used as an indicator for the activation of ATF6 or IRE1. In this work, sXBP1 was observed as an indicator for UPR mediated by both ATF6 and IRE1.

Data demonstrated herein demonstrate that the downregulation of the full-length Na⁺ channel and the upregulation of SCN5A variants E28C and E28D correlate with the increase of PERK induced by hypoxia or Ang II. However, siRNA PERK can partially reverse the downregulation of SCN5A full-length Na⁺ channel. Additionally, exogenous SCN5A variants E28C and E28D are introduced with E28C or E28D constructs. The results show that SCN5A variant E28C or E28D alone could induce the expression level of PERK. The corresponding expression changes of PERK, SCN5A and the variants E28C and E28D are similar between the endogenous SCN5A variants induced by hypoxia or Ang II and the exogenous SCN5A variants. This manifests that PERK is involved in the SCN5A downregulation mediated by SCN5A variants E28C and E28D. The results also demonstrate that SCN5A is downregulated in UPR-activated cells despite the sXBP1 inhibition. Negative results of sXBP1 may exclude the possibility that other two arms of UPR are involved in this downregulation. Therefore, PERK-mediated UPR is most likely to be the major pathway involved in the downregulation of full-length Na⁺ channel.

In the heart tissues, the UPR has been shown to play a role during development, hypertrophy, ischemia, and heart failure. Heart failure is associated with hypoxia, elevated Ang II, and increased catecholamines, all of which have been shown to activate the UPR.

Thus, the inhibition of UPR through PERK is an attractive target for heart failure therapy.

### Example 4

This example demonstrates that mRNA splicing abnormalities of the cardiac sodium channel gene (SCN5A) occurs in hypertrophic cardiomyopathy.

Hypertrophic cardiomyopathy (HCM) leading to sustained ventricular tachyarrhythmias is the most common cause of sudden cardiac death (SCD) in young patients. We have shown that adult patients with heart failure (HF) at increased risk of SCD have increased abundance of alternatively spliced mRNA for the cardiac voltage gated sodium channel (SCN5A) mRNA. These splice variants encode nonfunctional sodium channels that contribute to arrhythmic risk. We investigated if patients with HCM showed a similar increase in the abundance of abnormal SCN5A splice variants.

Human heart tissue was obtained from myectomy samples of 10 HCM patients along with cardiac tissue from 3 normal controls. Total RNA was extracted from the tissues and relative abundances of SCN5A, its variants, and the causative splicing factors RBM25, hLuc7a, and PRPF40A were determined by real-time RT-PCR.

HCM patients had an increased abundance of splice variant E28D compared to the hearts of normal controls, with an average 15±3 fold (p<0.05) Expressions of splicing factors RBM25 and PRPF40A were also increased 17±5 (p<0.05) and 11±4 fold (p<0.05) respectively along with a >1000 fold increase in HLuc7a in patients with HCM.

Cardiomyocytes from patients with HCM showed an increased abundance of an abnormal SCN5A mRNA splice variants along with increased expression of splicing factors when compared to the hearts of normal controls. The pattern of splicing was distinct from that reported in adult HF. These splice variants could contribute to the risk of arrhythmic sudden death in patients with HCM.

### Example 5

This example demonstrates the role of PERK-mediated unfolded protein response pathway in the regulation of cardiac sodium channel during human heart failure.

The unfolded protein response (UPR) is a series of interrelated signaling pathways that occur when the endoplasmic reticulum (ER) experiences excess secretory load, accumulates misfiled proteins, or is subject to other pathological conditions. UPR acts to attenuating general protein synthesis, induces the expression of ER chaperone proteins, and enhances the degradation of misfiled proteins. In published work, we have shown that heart failure (HF) increases alternative splicing of the SCN5A gene (encoding cardiac sodium channel), generating mRNA variants E28C and E28D encoding truncated, nonfunctional sodium channel. The presence of these variants causes a dominant negative downregulation of the wild-type SCN5A mRNA and sodium current to a sufficient extent to be arrthmogenic. We tested whether PERK-mediated UPR contributed to the dominant negative effect on Na⁺ current when truncated sodium channel mRNA variants are present in HF.

The correlation of expression changes among PERK, major human embryonic stem cell-derived cardiomyocytes (hESC-CMs). Hypoxia and Ang II were used as induces or abnormal splicing since they have been shown to mediate some of the pathological consequences of HF>hESC-CMs were divided into six experimental groups: normoxic, 1% O2 hypoxia treated, 100 nmol/L Ang II-treated, E28C overexpressed, E28D overexpressed, and empty vector control. E28C and E28D constructs were transduced to overexpress the truncated proteins.

The expression of major UPR components (PERK, calnexin, CHOP) were increased in HF tissues and cardiac Na⁺ channels were downregulated. In hESC-CMs, induction of SCN5A variants E28C and E28D with Ang II or hypoxia as well as expression of exogenous variants could induce major UPR components (PERK, calnexin, CHOP). Finally, downregualtion of PERK prevented the loss of full-length SCN5A mRNA abundance with these stimuli.

SCN5A variants could induce the expression of major UPR components and could induce PERK-mediated Na+ channed downregulation. The results indicate that the UPR contributes to Na+ channel downregulation during human HF.

Numerous modifications and variations in the practice of the invention are expected to occur to those of skill in the art upon consideration of the presently preferred embodiments thereof. Consequently, the only limitations which should be placed upon the scope of the invention are those which appear in the appended claims.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of identifying a subject at risk for arrhythmia or heart failure comprising the step of determining a level of splicing factor hLuc7a, splicing factor RBM25 and/or PERK in a biological sample obtained from the subject, wherein an increased level, compared to a control level, indicates a risk for arrhythmia or heart failure.

2. The method of claim 1, comprising determining an expression level of splicing factor hLuc7a, splicing factor RBM25, and/or PERK.

3. The method of claim 1, wherein the biological sample is a blood sample.

4. The method of claim 1, wherein the biological sample comprises cardiac tissue.

5. The method of claim 1, wherein the biological sample comprises, consists essentially of, or consists of white blood cells.

6. The method of claim 1, further comprising determining a level of a chaperone protein, a full-length SCN5A transcript, a SCN5A splice variant, or a combination thereof.

7. The method of claim 6, wherein the chaperone protein is CHOP or calnexin.

8. The method of claim 6, wherein the SCN5A splice variant is a splice variant produced from alternative splicing within Exon 28 of the SCN5A gene,

9. The method of claim 8, wherein the SCN5A splice variant SCN5A splice variant E28C or E28D.

10. The method of claim 1, wherein the method effectively identifies a subject at risk for sudden cardiac death.

11. The method of claim 1, wherein the method effectively determines whether the subject needs therapy for arrhythmia or for heart failure.

12. The method of claim 1, wherein the heart failure is a systolic heart failure.

13. A method of diagnosing hypertrophic cardiomyopathy (HCM), or a risk therefor, in a subject, comprising the step of determining a level of E28D, RBM25, PRPF40A, or LUC7L3, or a combination thereof, in a biological sample obtained from the subject, wherein an increased level is indicative of the subject having HCM or a risk therefor.

14. The method of claim 13, comprising determining an expression level of E28D, RBM25, PRPF40A, or LUC7L3, or a combination thereof.

15. The method of claim 13, wherein the biological sample is blood, comprise cardiac tissue, muscle tissue, or white blood cells.

16. A kit comprising two or more of:
a. an hLuc7A binding agent,
b. an RBM25 binding agent,
c. a PERK binding agent,
d. a PRPF40A binding agent,
e. a SCN5A splice variant E28D.
and instructions for use.

17. The kit of claim 16, comprising a combination of (a), (b), and (c) or a combination of (a), (b), (d), and (e).

18. The kit of claim 16, wherein the binding agent is an antibody or an antigen binding fragment thereof.

19. The kit of claim 16, wherein the binding agent is a nucleic acid probe that hybridizes to a nucleic acid encoding hLuc7A, RBM25, PERK, PRPF40A, or E28D.

20. The kit of claim 16, wherein the instructions comprise instructions for determining an expression level of hLuc7A, RBM25, PERK, PRPF40A, or E28D in a biological sample.


SEQUENCE LISTING (59 sequences)

SEQ ID NO: 1 - DNA, 335 bp, Homo sapiens
agtggacactgtgggcatgcgtgtccgtgcagcacatcgccgtgcaggagcctgggggaaggcctttccctcagtgagggttgcagcttccccacaggcaacgtgaggagagcctgtgcccagaagcaggatgagaagatggcaaacttcctattacctcggggcaccagcagcttccgcaggttcacacgggagtccctggcagccatcgagaagcgcatggcggagaagcaagcccgcggctcaaccaccttgcaggagagccgagaggggctgcccgaggaggaggctccccggccccagctggacctgcaggcctccaaaaagctgccaga

SEQ ID NO: 2 - DNA, 302 bp, Homo sapiens
acatcgccgtgcaggagcctgggggaaggcctttccctcagtgagggttgcagcttccccacaggcaacgtgaggagagcctgtgcccagaagcaggatgagaagatggcaaacttcctattacctcggggcaccagcagcttccgcaggttcacacgggagtccctggcagccatcgagaagcgcatggcggagaagcaagcccgcggctcaaccaccttgcaggagagccgagaggggctgcccgaggaggaggctccccggccccagctggacctgcaggcctccaaaaagctgccaga

SEQ ID NO: 3 - DNA, 301 bp, Homo sapiens
catcgccgtgcaggagcctgggggaaggcctttccctcagtgagggttgcagcttccccacaggcaacgtgaggagagcctgtgcccagaagcaggatgagaagatggcaaacttcctattacctcggggcaccagcagcttccgcaggttcacacgggagtccctggcagccatcgagaagcgcatggcggagaagcaagcccgcggctcaaccaccttgcaggagagccgagaggggctgcccgaggaggaggctccccggccccagctggacctgcaggcctccaaaaagctgccaga

SEQ ID NO: 4 - DNA, 298 bp, Homo sapiens
cgccgtgcaggagcctgggggaaggcctttccctcagtgagggttgcagcttccccacaggcaacgtgaggagagcctgtgcccagaagcaggatgagaagatggcaaacttcctattacctcggggcaccagcagcttccgcaggttcacacgggagtccctggcagccatcgagaagcgcatggcggagaagcaagcccgcggctcaaccaccttgcaggagagccgagaggggctgcccgaggaggaggctccccggccccagctggacctgcaggcctccaaaaagctgccaga

SEQ ID NO: 5 - DNA, 236 bp, Homo sapiens
aacgtgaggagagcctgtgcccagaagcaggatgagaagatggcaaacttcctattacctcggggcaccagcagcttccgcaggttcacacgggagtccctggcagccatcgagaagcgcatggcggagaagcaagcccgcggctcaaccaccttgcaggagagccgagaggggctgcccgaggaggaggctccccggccccagctggacctgcaggcctccaaaaagctgccaga

SEQ ID NO: 6 - DNA, 220 bp, Homo sapiens
gtgcccagaagcaggatgagaagatggcaaacttcctattacctcggggcaccagcagcttccgcaggttcacacgggagtccctggcagccatcgagaagcgcatggcggagaagcaagcccgcggctcaaccaccttgcaggagagccgagaggggctgcccgaggaggaggctccccggccccagctggacctgcaggcctccaaaaagctgccaga

SEQ ID NO: 7 - DNA, 321 bp, Homo sapiens
ggagccctcctagtgagtatgaagtgatatctcactgaggttttggtttgcaaaagcaaatgactgatgactaacgatgcaggacatctttccatgtgcatgttggtcatttatatatcttccttggagaaatctctattcagatccttagctcatttttaattgggttatttctcttcttcttgttgagttgtaagagttctttacatattctggatcacagtctcttatcagatatatgatttaaaaatattttctcctagtctgtgagttttttcatttcctagtggtgtccattaaagcacaaaagttttacatgtt

SEQ ID NO: 8 - DNA, 164 bp, Homo sapiens
gaactgcacaatgaccagcaggaggggagaagagagtaggaaaaaggagggaaggacagacatcaagtgccagatgttgtctgaactaatcgagcacttctcaccaaacttcatgtataaataaaatacatatttttaaaacaaaccaataaatggcttacatg

SEQ ID NO: 9 - DNA, 114 bp, Homo sapiens
ggcactgtgctctcggacatcatccagaagtacttcttctccccgacgctcttccgagtcatccgcctggcccgaataggccgcatcctcagactgatccgaggggccaagggg

SEQ ID NO: 10 - DNA, 410 bp, Homo sapiens
gaacagagccgcggagccgagacggcggcggcgcccgtaggatgcagggatcgctcccccggggccgctgagcctgcgcccagtgccccgagccccgcgccgagccgagtccgcgccaagcagcagccgcccaccccggggcccggccgggggaccagcagcttccccacaggcaacgtgaggagagcctgtgcccagaagcaggatgagaagatggcaaacttcctattacctcggggcaccagcagcttccgcaggttcacacgggagtccctggcagccatcgagaagcgcatggcggagaagcaagcccgcggctcaaccaccttgcaggagagccgagaggggctgcccgaggaggaggctccccggccccagctggacctgcaggcctccaaaaagctgccaga

SEQ ID NO: 11 - DNA, 250 bp, Homo sapiens
gcttccccacaggcaacgtgaggagagcctgtgcccagaagcaggatgagaagatggcaaacttcctattacctcggggcaccagcagcttccgcaggttcacacgggagtccctggcagccatcgagaagcgcatggcggagaagcaagcccgcggctcaaccaccttgcaggagagccgagaggggctgcccgaggaggaggctccccggccccagctggacctgcaggcctccaaaaagctgccaga

SEQ ID NO: 12 - DNA, 2066 bp, Homo sapiens
ggcactgtgctctcggacatcatccagaagtacttcttctccccgacgctcttccgagtcatccgcctggcccgaataggccgcatcctcagactgatccgaggggccaaggggatccgcacgctgctctttgccctcatgatgtccctgcctgccctcttcaacatcgggctgctgctcttcctcgtcatgttcatctactccatctttggcatggccaacttcgcttatgtcaagtgggaggctggcatcgacgacatgttcaacttccagaccttcgccaacagcatgctgtgcctcttccagatcaccacgtcggccggctgggatggcctcctcagccccatcctcaacactgggccgccctactgcgaccccactctgcccaacagcaatggctctcggggggactgcgggagcccagccgtgggcatcctcttcttcaccacctacatcatcatctccttcctcatcgtggtcaacatgtacattgccatcatcctggagaacttcagcgtggccacggaggagagcaccgagcccctgagtgaggacgacttcgatatgttctatgagatctgggagaaatttgacccagaggccactcagtttattgagtattcggtcctgtctgactttgccgacgccctgtctgagccactccgtatcgccaagcccaaccagataagcctcatcaacatggacctgcccatggtgagtggggaccgcatccattgcatggacattctctttgccttcaccaaaagggtcctgggggagtctggggagatggacgccctgaagatccagatggaggagaagttcatggcagccaacccatccaagatctcctacgagcccatcaccaccacactccggcgcaagcacgaagaggtgtcggccatggttatccagagagccttccgcaggcacctgctgcaacgctctttgaagcatgcctccttcctcttccgtcagcaggcgggcagcggcctctccgaagaggatgcccctgagcgagagggcctcatcgcctacgtgatgagtgagaacttctcccgaccccttggcccaccctccagctcctccatctcctccacttccttcccaccctcctatgacagtgtcactagagccaccagcgataacctccaggtgcgggggtctgactacagccacagtgaagatctcgccgacttccccccttctccggacagggaccgtgagtccatcgtgtgagcctcggcctggctggccaggacacactgaaaagcagcctttttcaccatggcaaacctaaatgcagtcagtcacaaaccagcctggggccttcctggctttgggagtaagaaatgggcctcagccccgcggatcaaccaggcagagttctgtggcgccgcgtggacagccggagcagttggcctgtgcttggaggcctcagatagacctgtgacctggtctggtcaggcaatgccctgcggctctggaaagcaacttcatcccagctgctgaggcgaaatataaaactgagactgtatatgttgtgaatgggctttcataaatttattatatttgatatttttttacttgagcaaagaactaaggatttttccatggacatgggcagcaattcacgctgtctcttcttaaccctgaacaagagtgtctatggagcagccggaagtctgttctcaaagcagaagtggaatccagtgtggctcccacaggtcttcactgcccaggggtcgaatggggtccccctcccacttgacctgagatgctgggagggctgaacccccactcacacaagcacacacacacagtcctcacacacggaggccagacacaggccgtgggacccaggctcccagcctaagggagacaggcctttccctgccggccccccaaggatggggttcttgtccacggggctcactctggccccctattgtctccaaggtcccattttccccctgtgttttcacgcaggtcatattgtcagtcctacaaaaataaaaggcttccagaggagagtggcctgggtcccagggctggccctaggcact

SEQ ID NO: 13 - DNA, 3497 bp, Homo sapiens
ggcactgtgctctcggacatcatccagaagtacttcttctccccgacgctcttccgagtcatccgcctggcccgaataggccgcatcctcagactgatccgaggggccaaggggatccgcacgctgctctttgccctcatgatgtccctgcctgccctcttcaacatcgggctgctgctcttcctcgtcatgttcatctactccatctttggcatggccaacttcgcttatgtcaagtgggaggctggcatcgacgacatgttcaacttccagaccttcgccaacagcatgctgtgcctcttccagatcaccacgtcggccggctgggatggcctcctcagccccatcctcaacactgggccgccctactgcgaccccactctgcccaacagcaatggctctcggggggactgcgggagcccagccgtgggcatcctcttcttcaccacctacatcatcatctccttcctcatcgtggtcaacatgtacattgccatcatcctggagaacttcagcgtggccacggaggagagcaccgagcccctgagtgaggacgacttcgatatgttctatgagatctgggagaaatttgacccagaggccactcagtttattgagtattcggtcctgtctgactttgccgacgccctgtctgagccactccgtatcgccaagcccaaccagataagcctcatcaacatggacctgcccatggtgagtggggaccgcatccattgcatggacattctctttgccttcaccaaaagggtcctgggggagtctggggagatggacgccctgaagatccagatggaggagaagttcatggcagccaacccatccaagatctcctacgagcccatcaccaccacactccggcgcaagcacgaagaggtgtcggccatggttatccagagagccttccgcaggcacctgctgcaacgctctttgaagcatgcctccttcctcttccgtcagcaggcgggcagcggcctctccgaagaggatgcccctgagcgagagggcctcatcgcctacgtgatgagtgagaacttctcccgaccccttggcccaccctccagctcctccatctcctccacttccttcccaccctcctatgacagtgtcactagagccaccagcgataacctccaggtgcgggggtctgactacagccacagtgaagatctcgccgacttccccccttctccggacagggaccgtgagtccatcgtgtgagcctcggcctggctggccaggacacactgaaaagcagcctttttcaccatggcaaacctaaatgcagtcagtcacaaaccagcctggggccttcctggctttgggagtaagaaatgggcctcagccccgcggatcaaccaggcagagttctgtggcgccgcgtggacagccggagcagttggcctgtgcttggaggcctcagatagacctgtgacctggtctggtcaggcaatgccctgcggctctggaaagcaacttcatcccagctgctgaggcgaaatataaaactgagactgtatatgttgtgaatgggctttcataaatttattatatttgatatttttttacttgagcaaagaactaaggatttttccatggacatgggcagcaattcacgctgtctcttcttaaccctgaacaagagtgtctatggagcagccggaagtctgttctcaaagcagaagtggaatccagtgtggctcccacaggtcttcactgcccaggggtcgaatggggtccccctcccacttgacctgagatgctgggagggctgaacccccactcacacaagcacacacacacagtcctcacacacggaggccagacacaggccgtgggacccaggctcccagcctaagggagacaggcctttccctgccggccccccaaggatggggttcttgtccacggggctcactctggccccctattgtctccaaggtcccattttccccctgtgttttcacgcaggtcatattgtcagtcctacaaaaataaaaggcttccagaggagagtggcctgggtcccagggctggccctaggcactgatagttgccttttcttcccctcctgtaagagtattaacaaaaccaaaggacacaagggtgcaagccccattcacggcctggcatgcagcttgtccttgctcctggaacctggcaggccctgcccagccagccatcggaagagagggctgagccatgggggtttggggctaagaagttcaccagccctgagccatggcggcccctcagcctgcctgaagagaggaaactggcgatctcccagggctctctggaccatacgcggaggagttttctgtgtggtctccagctcctctccagacacagagacatgggagtggggagcggagcttggccctgcgccctgtgcagggaaagggatggtcaggcccagttctcgtgcccttagaggggaatgaaccatggcacctttgagagagggggcactgtggtcaggcccagcctctctggctcagcccgggatcctgatggcacccacacagaggacctctttggggcaagatccaggtggtcccataggtcttgtgaaaaggctttttcagggaaaaatattttactagtccaatcacccccaggacctcttcagctgctgacaatcctatttagcatatgcaaatcttttaacatagagaactgtcaccctgaggtaacagggtcaactggcgaagcctgagcaggcaggggcttggctgccccattccagctctcccatggagcccctccaccgggcgcatgcctcccaggccacctcagtctcacctgccggctctgggctggctgctcctaacctacctcgccgagctgtcggagggctggacatttgtggcagtgctgaagggggcattgccggcgagtaaagtattatgtttcttcttgtcaccccagttcccttggtggcaaccccagacccaacccatgcccctgacagatctagttctcttctcctgtgttccctttgagtccagtgtgggacacggtttaactgtcccagcgacatttctccaagtggaaatcctatttttgtagatctccatgctttgctctcaaggcttggagaggtatgtgcccctcctgggtgctcaccgcctgctacacaggcaggaatgcggttgggaggcaggtcgggctgccagcccagctggccggaaggagactgtggtttttgtgtgtgtggacagcccgggagctttgagacaggtgcctggggctggctgcagacggtgtggttgggggtgggaggtgagctagacccaacccttagcttttagcctggctgtcacctttttaatttccagaactgcacaatgaccagcaggagggaaggacagacatcaagtgccagatgttgtctgaactaatcgagcacttctcaccaaacttcatgtataaataaaatacatatttttaaaacaaaccaataaatggcttacatg

SEQ ID NO: 14 - protein, 412 aa, Homo sapiens
GTVLSDIIQKYFFSPTLFRVIRLARIGRILRLIRGAKGIRTLLFALMMSLPALFNIGLLLFLVMFIYSIFGMANFAYVKWEAGIDDMFNFQTFANSMLCLFQITTSAGWDGLLSPILNTGPPYCDPTLPNSNGSRGDCGSPAVGILFFTTYIIISFLIVVNMYIAIILENFSVATEESTEPLSEDDFDMFYEIWEKFDPEATQFIEYSVLSDFADALSEPLRIAKPNQISLINMDLPMVSGDRIHCMDILFAFTKRVLGESGEMDALKIQMEEKFMAANPSKISYEPITTTLRRKHEEVSAMVIQRAFRRHLLQRSLKHASFLFRQQAGSGLSEEDAPEREGLIAYVMSENFSRPLGPPSSSSISSTSFPPSYDSVTRATSDNLQVRGSDYSHSEDLADFPPSPDRDRESIV

SEQ ID NO: 15 - protein, 8 aa, Homo sapiens
GALLVSMK

SEQ ID NO: 16 - protein, 12 aa, Homo sapiens
ELHNDQQEGRRE

SEQ ID NO: 17 - protein, 128 aa, Homo sapiens
NKYQGFIFDIVTKQAFDVTIMFLICLNMVTMMVETDDQSPEKINILAKINLLFVAIFTGECIVKLAALRHYYFTNSWNIFDFVVVILSIVGTVLSDIIQKYFFSPTLFRVIRLARIGRILRLIRGAKG

SEQ ID NO: 18 - DNA, 22 bp, Artificial Sequence
  Synthetic primer
ttacgcaccttccgagtcctcc

SEQ ID NO: 19 - DNA, 22 bp, Artificial Sequence
  Synthetic primer
gatgagggcaaagacgctgagg

SEQ ID NO: 20 - DNA, 24 bp, Artificial Sequence
  Synthetic primer
tctcttctcccctcctgctggtca

SEQ ID NO: 21 - DNA, 25 bp, Artificial Sequence
  Synthetic primer
ggaagagcgtcggggagaagaagta

SEQ ID NO: 22 - DNA, 20 bp, Artificial Sequence
  Synthetic primer
agtctctgctggagtcttca

SEQ ID NO: 23 - DNA, 22 bp, Artificial Sequence
  Synthetic primer
tgacactgtgtctcagactctt

SEQ ID NO: 24 - DNA, 25 bp, Artificial Sequence
  Synthetic primer
tgtcttttccacctcatttgaatcg

SEQ ID NO: 25 - DNA, 23 bp, Artificial Sequence
  Synthetic primer
attggtacaggaatcattggggt

SEQ ID NO: 26 - DNA, 25 bp, Artificial Sequence
  Synthetic primer
ggaccaagatcagaacgtgtatttg

SEQ ID NO: 27 - DNA, 24 bp, Artificial Sequence
  Synthetic primer
cagttgttggatgagttaatgggc

SEQ ID NO: 28 - DNA, 20 bp, Artificial Sequence
  Synthetic primer
tctgctgagtccgcagcagg

SEQ ID NO: 29 - DNA, 20 bp, Artificial Sequence
  Synthetic primer
ctctaagactagaggcttgg

SEQ ID NO: 30 - DNA, 16 bp, Artificial Sequence
  Synthetic primer
ggatcggcggctccat

SEQ ID NO: 31 - DNA, 22 bp, Artificial Sequence
  Synthetic primer
catactcctgcttgctgatcca

SEQ ID NO: 32 - RNA, 20 nt, Artificial Sequence
  RNA substrate
cagcaggcgggcagcggccu

SEQ ID NO: 33 - RNA, 20 nt, Artificial Sequence
  RNA substrate
cagcagguuagaggcggccu

SEQ ID NO: 34 - DNA, 2020 bp, Homo sapiens
  ref <308>: DATABASE ACCESSION NUMBER: NCBI / NM_006107.3
  ref <309>: DATABASE ENTRY DATE: 2011-08-14
  ref <313>: RELEVANT RESIDUES IN SEQ ID NO: (1)..(2020)
acggcatgctgggaaggcgtccgcgcggcggccattttgtcttgtcggctcctgtgtgtaggagggatttcggcctgagagcgggccgaggagattggcgacggtgtcgcccgtgttttcgttggcgggtgcctgggctggtgggaacagccgcccgaaggaagcaccatgatttcggccgcgcagttgttggatgagttaatgggccgggaccgaaacctagccccggacgagaagcgcagcaacgtgcggtgggaccacgagagcgtttgtaaatattatctctgtggtttttgtcctgcggaattgttcacaaatacacgttctgatcttggtccgtgtgaaaaaattcatgatgaaaatctacgaaaacagtatgagaagagctctcgtttcatgaaagttggctatgagagagattttttgcgatacttacagagcttacttgcagaagtagaacgtaggatcagacgaggccatgctcgtttggcattatctcaaaaccagcagtcttctggggccgctggcccaacaggcaaaaatgaagaaaaaattcaggttctaacagacaaaattgatgtacttctgcaacagattgaagaattagggtctgaaggaaaagtagaagaagcccaggggatgatgaaattagttgagcaattaaaagaagagagagaactgctaaggtccacaacgtcgacaattgaaagctttgctgcacaagaaaaacaaatggaagtttgtgaagtatgtggagcctttttaatagtaggagatgcccagtcccgggtagatgaccatttgatgggaaaacaacacatgggctatgccaaaattaaagctactgtagaagaattaaaagaaaagttaaggaaaagaaccgaagaacctgatcgtgatgagcgtctaaaaaaggagaagcaagaaagagaagaaagagaaaaagaacgggagagagaaagggaagaaagagaaaggaaaagacgaagggaagaggaagaaagagaaaaagaaagggctcgtgacagagaaagaagaaagagaagtcgttcacgaagtagacactcaagccgaacatcagacagaagatgcagcaggtctcgggaccacaaaaggtcacgaagtagagaaagaaggcggagcagaagtagagatcgacgaagaagcagaagccatgatcgatcagaaagaaaacacagatctcgaagtcgggatcgaagaagatcaaaaagccgggatcgaaagtcatataagcacaggagcaaaagtcgggacagagaacaagatagaaaatccaaggagaaagaaaagaggggatctgatgataaaaaaagtagtgtgaagtccggtagtcgagaaaagcagagtgaagacacaaacactgaatcgaaggaaagtgatactaagaatgaggtcaatgggaccagtgaagacattaaatctgaaggtgacactcagtccaattaaaactgatctgataagacctcagatcagacagaggactactgttcgaagatttttggaagaatactgagaacggcataaagtgaagatcgacatttaaaaaatgaggtgaaagaaagctatagtggcatagaaaaagtataaagctcagttagtttttttattattattattattaaaagttaattcaggactgatgtgacctaccagatttcagaacatgtgttaatagtatatatgccactgaaaacttaggtcctgtatcatacttttttctttaagactttttaagaaatattacttaaacatgtggcttgctcagtgtttaattgcaagttttcaatcttggactttgaaaacaggattaaacgttagtattcgtgtgaatcagactaagtgggatttcatttttacaactctgctctacttagcctttggatttagaagtaaaaataaagtatctctgactttctgttacaaagttgattgtctctgtcattgaaaagttttagtattaatctttttctaataaagttattgactctgaaaaaaaaaa

SEQ ID NO: 35 - DNA, 3525 bp, Homo sapiens
  ref <308>: DATABASE ACCESSION NUMBER: NCBI / NM_016424.4
  ref <309>: DATABASE ENTRY DATE: 2011-08-14
  ref <313>: RELEVANT RESIDUES IN SEQ ID NO: (1)..(3525)
acggcatgctgggaaggcgtccgcgcggcggccattttgtcttgtcggctcctgtgtgtaggagggatttcggcctgagagcgggccgaggagattggcgacggtgtcgcccgtgttttcgttggcgggtgcctgggctggtgggaacagccgcccgaaggaagcaccatgatttcggccgcgcagttgttggatgagttaatgggccgggaccgaaacctagccccggacgagaagcgcagcaacgtgcggtgggaccacgagagcgtttgtaaatattatctctgtggtttttgtcctgcggaattgttcacaaatacacgttctgatcttggtccgtgtgaaaaaattcatgatgaaaatctacgaaaacagtatgagaagagctctcgtttcatgaaagttggctatgagagagattttttgcgatacttacagagcttacttgcagaagtagaacgtaggatcagacgaggccatgctcgtttggcattatctcaaaaccagcagtcttctggggccgctggcccaacaggcaaaaatgaagaaaaaattcaggttctaacagacaaaattgatgtacttctgcaacagattgaagaattagggtctgaaggaaaagtagaagaagcccaggggatgatgaaattagttgagcaattaaaagaagagagagaactgctaaggtccacaacgtcgacaattgaaagctttgctgcacaagaaaaacaaatggaagtttgtgaagtatgtggagcctttttaatagtaggagatgcccagtcccgggtagatgaccatttgatgggaaaacaacacatgggctatgccaaaattaaagctactgtagaagaattaaaagaaaagttaaggaaaagaaccgaagaacctgatcgtgatgagcgtctaaaaaaggagaagcaagaaagagaagaaagagaaaaagaacgggagagagaaagggaagaaagagaaaggaaaagacgaagggaagaggaagaaagagaaaaagaaagggctcgtgacagagaaagaagaaagagaagtcgttcacgaagtagacactcaagccgaacatcagacagaagatgcagcaggtctcgggaccacaaaaggtcacgaagtagagaaagaaggcggagcagaagtagagatcgacgaagaagcagaagccatgatcgatcagaaagaaaacacagatctcgaagtcgggatcgaagaagatcaaaaagccgggatcgaaagtcatataagcacaggagcaaaagtcgggacagagaacaagatagaaaatccaaggagaaagaaaagaggggatctgatgataaaaaaagtagtgtgaagtccggtagtcgagaaaagcagagtgaagacacaaacactgaatcgaaggaaagtgatactaagaatgaggtcaatgggaccagtgaagacattaaatctgaaggtgacactcagtccaattaaaactgatctgataagacctcagatcagacagaggtaagtgtattgtttctcactttgattagggctttttgttactgtttgacagtgcagcgtaagtatgcacagatgaagatggaactaagccgagtaagaagacatacaaaagcctcttctgaaggaaaagacagtgtagtcctgcaaaacattttgaggtacattgttttgtctcagctattttgtagcagactcgtgcccccattagtgtgcctctttggaaattatcgcccacatttgtaatatagtcgccattgaaaagttaattatcctttttttagggattttgatgtcatttcttttttttttttaataaaaaggttgaactgtttttttttttctttttggtattaagtccatcttgtgttggtacattggcagagacatatgctttaaaaacttaaatatttcggaggcacatgttggactactttgttttaattaaactgctagtatttctttgtcaaggatgtttctagttttttgctttattgccttgcattctaatgcagtttgttctgtaactcgagagccagtagcattggattgatggaagtgtagggtttatgaattattgcagctgactaccatacctcacacagcgttggtgttgtgagcggcccatgaaaagccaaattaaaaatcaaggattcagtcaaactaagcaggtactcatgccaggtactcctttctctacccacatccatgtttgaatgctattgcctgtgatctttacgcttaactgttgtgtatcttttttgttctttacaagaagtgcagaggggttttttgtgtattgcgtgaaaacttataaaacaaatgttaacagaatggaattttttttcaactgtatgtagggctgcagtggtggccagaattagatatctttaaagaattttaaatacaataaacacttcatattattcgccttgttacactcaatgcaattctcaagtctataagaggtatgtgcttaatatttcctactgtgtaggagaatttgcagtcagccataggtatgtaggaatagtcactcactggctgatacatttaaagcagcagtgtgaatagcaaggacagacaccttcaatttgtgaaatcaaagaactgatgcactatatagaacgaatttgggtttttaaagaaatattaaaagttaggtactgtaagtgttcttaaaacctgtaaacttcattctgtgggctagtggtgtgggacaaaatattcctaatgaaaggaagtaccaattagttgatttgttggtggcattccccttttgggaaagcaatgtaaggttatgtctgtgtatgtcattcacacttaggcaagcatacacaggcacatggctttaagaaccacactgatgccttgataattaaaaagaatacaagcattccatgtacacatgttaattagcagttagtgactgggccaacactttctcataaaaattggccttttacatgttgtctaattatcatttttccccaaattttgcgttgtaggactactgttcgaagatttttggaagaatactgagaacggcataaagtgaagatcgacatttaaaaaatgaggtgaaagaaagctatagtggcatagaaaaagtataaagctcagttagtttttttattattattattattaaaagttaattcaggactgatgtgacctaccagatttcagaacatgtgttaatagtatatatgccactgaaaacttaggtcctgtatcatacttttttctttaagactttttaagaaatattacttaaacatgtggcttgctcagtgtttaattgcaagttttcaatcttggactttgaaaacaggattaaacgttagtattcgtgtgaatcagactaagtgggatttcatttttacaactctgctctacttagcctttggatttagaagtaaaaataaagtatctctgactttctgttacaaagttgattgtctctgtcattgaaaagttttagtattaatctttttctaataaagttattgactctgaaaaaaaaaa

SEQ ID NO: 36 - protein, 432 aa, Homo sapiens
  ref <308>: DATABASE ACCESSION NUMBER: NCBI / NP_006098.2
  ref <309>: DATABASE ENTRY DATE: 2011-08-14
  ref <313>: RELEVANT RESIDUES IN SEQ ID NO: (1)..(432)
MISAAQLLDELMGRDRNLAPDEKRSNVRWDHESVCKYYLCGFCPAELFTNTRSDLGPCEKIHDENLRKQYEKSSRFMKVGYERDFLRYLQSLLAEVERRIRRGHARLALSQNQQSSGAAGPTGKNEEKIQVLTDKIDVLLQQIEELGSEGKVEEAQGMMKLVEQLKEERELLRSTTSTIESFAAQEKQMEVCEVCGAFLIVGDAQSRVDDHLMGKQHMGYAKIKATVEELKEKLRKRTEEPDRDERLKKEKQEREEREKEREREREERERKRRREEEEREKERARDRERRKRSRSRSRHSSRTSDRRCSRSRDHKRSRSRERRRSRSRDRRRSRSHDRSERKHRSRSRDRRRSKSRDRKSYKHRSKSRDREQDRKSKEKEKRGSDDKKSSVKSGSREKQSEDTNTESKESDTKNEVNGTSEDIKSEGDTQSN

SEQ ID NO: 37 - protein, 432 aa, Homo sapiens; identical to SEQ ID NO: 36
  ref <308>: DATABASE ACCESSION NUMBER: NCBI / NP_057508.2
  ref <309>: DATABASE ENTRY DATE: 2011-08-14
  ref <313>: RELEVANT RESIDUES IN SEQ ID NO: (1)..(432)

SEQ ID NO: 38 - DNA, 4303 bp, Homo sapiens
  ref <308>: DATABASE ACCESSION NUMBER: NCBI / NM_021239.2
  ref <309>: DATABASE ENTRY DATE: 2011-08-14
  ref <313>: RELEVANT RESIDUES IN SEQ ID NO: (1)..(4303)
agtgcacgccgggcaagaggaagacctccatcagctcgccgcgcagcgcggctgtatttgcggcctgtgcgagtaggcgcttgggcactcagtctccctggcgagcgacgggcagaaatctcgaaccagtggagcgcactcgtaacctggatcccagaaggtcgcgaaggcagtaccgtttcctcagcggcggactgctgcagtaagaatgtcttttccacctcatttgaatcgccctcccatgggaatcccagcactcccaccagggatcccacccccgcagtttccaggatttcctccacctgtacctccagggaccccaatgattcctgtaccaatgagcattatggctcctgctccaactgtcttagtacccactgtgtctatggttggaaagcatttgggcgcaagaaaggatcatccaggcttaaaggctaaagaaaatgatgaaaattgtggtcctactaccactgtttttgttggcaacatttccgagaaagcttcagacatgcttataagacaactcttagctaaatgtggtttggttttgagctggaagagagtacaaggtgcttccggaaagcttcaagccttcggattctgtgagtacaaggagccagaatctaccctccgtgcactcagattattacatgacctgcaaattggagagaaaaagctactcgttaaagttgatgcaaagacaaaggcacagctggatgaatggaaagcaaagaagaaagcttctaatgggaatgcaaggccagaaactgtcactaatgacgatgaagaagccttggatgaagaaacaaagaggagagatcagatgattaaaggggctattgaagttttaattcgtgaatactccagtgagctaaatgccccctcacaggaatctgattctcaccccaggaagaagaagaaggaaaagaaggaggacattttccgcagatttccagtggccccactgatcccttatccactcatcactaaggaggatataaatgctatagaaatggaagaagacaaaagagacctgatatctcgagagatcagcaaattcagagacacacataagaaactggaagaagagaaaggcaaaaaggaaaaagaaagacaggaaattgagaaagaacggagagaaagagagagggagcgtgaaagggaacgagaaaggcgagaacgggaacgagaaagggaaagagaacgtgaacgagaaaaggagaaagaacgggagcgggaacgagaacgggatagggaccgtgaccggacaaaagagagagaccgagatcgggatcgagagagagatcgtgaccgggatagagaaaggagctcagatcgtaataaggatcgcagtcgatcaagagaaaaaagcagagatcgtgaaagggaacgagagcgggaaagagagagagagagagaacgagagcgagaacgagaacgggagcgagagagagagcgagagagggaacgggagcgagaaagagaaaaagacaaaaaacgggaccgagaagaagatgaagaagatgcatacgaacgaagaaaacttgaaagaaaactccgagagaaagaagctgcttatcaagagcgccttaagaattgggaaatcagagaacgaaagaaaacccgggaatatgagaaagaagctgaaagagaagaagaaagaagaagagaaatggccaaagaagctaaacgactaaaagaattcttagaagactatgatgatgatagagatgaccccaaatattacagaggaagtgctcttcagaaaaggttgcgtgatagagaaaaggaaatggaagcagatgaacgagataggaagagagagaaggaggagcttgaggaaatcaggcagcgccttctggcagaagggcatccagatccagatgcagagctccagaggatggaacaagaggctgagaggcgcaggcagccacaaataaagcaagagccagaatcagaagaggaggaagaagaaaagcaagaaaaagaagaaaaacgagaagaacccatggaagaggaagaggagccagagcaaaagccttgtctgaaacctactctgaggcccatcagctctgctccatctgtttcctctgccagtggcaatgcaacacctaacactcctggggatgagtctccctgtggtattattattcctcatgaaaactcaccagatcaacagcaacctgaggagcataggccaaaaataggactaagtcttaaactgggtgcttccaatagtcctggtcagcctaattctgtgaagagaaagaaactacctgtagatagtgtctttaacaaatttgaggatgaagacagtgatgacgtaccccgaaaaaggaaactggttcccttggattatggtgaagatgataaaaatgcaaccaaaggcactgtaaacactgaagaaaagcgtaaacacattaagagtctcattgagaaaatccctacagccaaacctgagctcttcgcttatcccctggattggtctattgtggattctatactgatggaacgtcgaattagaccatggattaataagaaaatcatagaatatataggtgaagaagaagctacattagttgattttgtttgttctaaggttatggctcatagttcaccccagagcattttagatgatgttgccatggtacttgatgaagaagcagaagtttttatagtcaaaatgtggagattattgatatatgaaacagaagccaagaaaattggtcttgtgaagtaaaactttttatatttagagttccatttcagatttcttctttgccacccttttaaggactttgaatttttctttgtctttgaagacattgtgagatctgtaattttttttttttgtagaaaatgtgaattttttggtcctctaatttgttgttgccctgtgtactcccttggttgtaaagtcatctgaatccttggttctctttatactcaccaggtacaaattactggtatgttttataagccgcagctactgtacacagcctatctgatataatcttgttctgctgatttgtttcttgtaaatattaaaacgactccccaattattttgcagaattgcacttaatattgaaatgtactgtataggaaccaacatgaacaattttaattgaaaacaccagtcataaactattaccacccccactctcttttgatcagaaatggcaagcccttgtgaaggcatggagtttaaaattggaatgcaaaaattagcagacaatccattcctactgtatttctgtatgaatgtgtttgtgaatgtatgtgtaaaagtctttcttttccctaatttgctttggtggggtccttaaaacatttcccaactaaagaatagaattgtaaaggaaaagtggtactgttccaacctgaaatgtctgttataattaggttattagtttcccagagcatggtgttctcgtgtcgtgagcaatgtggtttgctaactggatggggttttcttattaataagatggctgcttcagcttctcttttaaaggaatgtggatcatagtgatttttccttttaattttattgctcagaaatgaggcatatcctaaaaatcctggagagctgtatttaatgcatttttgcactaattggtccttagtttaattctattgtatctgtttatttaacaaaaaattcatcataccaaaaagtgtaagtgaaaaccccctttaaaacaaaacaaaaaaatgaaataaaattaggcaaattgacagacagtgagagttttacaaacatgataggtattctgctcggcaatttgtaagtttacatgttatttaaggataaaggtaaatcattcaaggcagttaccaaccactaactatttgttttcatttttgtcttgtagaaggtttatatcttgttttaccttggctcattagtgtttaaaaatgtactgatgatgtgcttagagaaattcctggggctttcttcgttgtagatcagaatttcaccagggagtaaaattacctgaaaacgtaagaagttttaaacagcttttcacacaaattagatgcaactgttcccatgtctgagtacttatttaaaagaaaggtaaagattggcctgttagaaaaagcataatgtgagctttggattactggattttttttttttttaaacacacctggagaggacatttgaaaacactgttcttaccctcgaaccctgatgtggttccattatgtaaatatttcaaatattaaaaatgtatatatttgatcctggggactcatattctttcagaatcatgtaaataaatggcatcatgttgtaa

SEQ ID NO: 39 - protein, 843 aa, Homo sapiens
  ref <308>: DATABASE ACCESSION NUMBER: NCBI / NP_067062.1
  ref <309>: DATABASE ENTRY DATE: 2010-08-14
  ref <313>: RELEVANT RESIDUES IN SEQ ID NO: (1)..(843)
MSFPPHLNRPPMGIPALPPGIPPPQFPGFPPPVPPGTPMIPVPMSIMAPAPTVLVPTVSMVGKHLGARKDHPGLKAKENDENCGPTTTVFVGNISEKASDMLIRQLLAKCGLVLSWKRVQGASGKLQAFGFCEYKEPESTLRALRLLHDLQIGEKKLLVKVDAKTKAQLDEWKAKKKASNGNARPETVTNDDEEALDEETKRRDQMIKGAIEVLIREYSSELNAPSQESDSHPRKKKKEKKEDIFRRFPVAPLIPYPLITKEDINAIEMEEDKRDLISREISKFRDTHKKLEEEKGKKEKERQEIEKERRERERERERERERREREREREREREREKEKERERERERDRDRDRTKERDRDRDRERDRDRDRERSSDRNKDRSRSREKSRDREREREREREREREREREREREREREREREREREREKDKKRDREEDEEDAYERRKLERKLREKEAAYQERLKNWEIRERKKTREYEKEAEREEERRREMAKEAKRLKEFLEDYDDDRDDPKYYRGSALQKRLRDREKEMEADERDRKREKEELEEIRQRLLAEGHPDPDAELQRMEQEAERRRQPQIKQEPESEEEEEEKQEKEEKREEPMEEEEEPEQKPCLKPTLRPISSAPSVSSASGNATPNTPGDESPCGIIIPHENSPDQQQPEEHRPKIGLSLKLGASNSPGQPNSVKRKKLPVDSVFNKFEDEDSDDVPRKRKLVPLDYGEDDKNATKGTVNTEEKRKHIKSLIEKIPTAKPELFAYPLDWSIVDSILMERRIRPWINKKIIEYIGEEEATLVDFVCSKVMAHSSPQSILDDVAMVLDEEAEVFIVKMWRLLIYETEAKKIGLVK

SEQ ID NO: 40 - DNA, 4665 bp, Homo sapiens
  ref <308>: DATABASE ACCESSION NUMBER: NCBI / NM_004836.5
  ref <309>: DATABASE ENTRY DATE: 2011-10-16
  ref <313>: RELEVANT RESIDUES IN SEQ ID NO: (1)..(4665)
ggaaagtccaccttccccaacaaggccagcctgggaacatggagtggcagcggccgcagccaatgagagagcaaacgcgcggaaagtttgctcaatgggcgatgtccgagataggctgtcactcaggtggcagcggcagaggccgggctgagacgtggccaggggaacacggctggctgtccaggccgtcggggcggcagtagggtccctagcacgtccttgccttcttgggagctccaagcggcgggagaggcaggcgtcagtggctgcgcctccatgcctgcgcgcggggcgggacgctgatggagcgcgccatcagcccggggctgctggtacgggcgctgctgctgctgctgctgctgctggggctcgcggcaaggacggtggccgcggggcgcgcccgtggcctcccagcgccgacggcggaggcggcgttcggcctcggggcggccgctgctcccacctcagcgacgcgagtaccggcggcgggcgccgtggctgcggccgaggtgactgtggaggacgctgaggcgctgccggcagccgcgggagagcaggagcctcggggtccggaaccagacgatgagacagagttgcgaccgcgcggcaggtcattagtaattatcagcactttagatgggagaattgctgccttggatcctgaaaatcatggtaaaaagcagtgggatttggatgtgggatccggttccttggtgtcatccagccttagcaaaccagaggtatttgggaataagatgatcattccttccctggatggagccctcttccagtgggaccaagaccgtgaaagcatggaaacagttcctttcacagttgaatcacttcttgaatcttcttataaatttggagatgatgttgttttggttggaggaaaatctctgactacatatggactcagtgcatatagtggaaaggtgaggtatatctgttcagctctgggttgtcgccaatgggatagtgacgaaatggaacaagaggaagacatcctgcttctacagcgtacccaaaaaactgttagagctgtcggacctcgcagtggcaatgagaagtggaatttcagtgttggccactttgaacttcggtatattccagacatggaaacgagagccggatttattgaaagcacctttaagcccaatgagaacacagaagagtctaaaattatttcagatgtggaagaacaggaagctgccataatggacatagtgataaaggtttcggttgctgactggaaagttatggcattcagtaagaagggaggacatctggaatgggagtaccagttttgtactccaattgcatctgcctggttacttaaggatgggaaagtcattcccatcagtctttttgatgatacaagttatacatctaatgatgatgttttagaagatgaagaagacattgtagaagctgccagaggagccacagaaaacagtgtttacttgggaatgtatagaggccagctgtatctgcagtcatcagtcagaatttcagaaaagtttccttcaagtcccaaggctttggaatctgtcactaatgaaaacgcaattattcctttaccaacaatcaaatggaaacccttaattcattctccttccagaactcctgtcttggtaggatctgatgaatttgacaaatgtctcagtaatgataagttttctcatgaagaatatagtaatggtgcactttcaatcttgcagtatccatatgataatggttattatctaccatactacaagagggagaggaacaaacgaagcacacagattacagtcagattcctcgacaacccacattacaacaagaatatccgcaaaaaggatcctgttcttcttttacactggtggaaagaaatagttgcaacgattttgttttgtatcatagcaacaacgtttattgtgcgcaggcttttccatcctcatcctcacaggcaaaggaaggagtctgaaactcagtgtcaaactgaaaataaatatgattctgtaagtggtgaagccaatgacagtagctggaatgacataaaaaactctggatatatatcacgatatctaactgattttgagccaattcaatgcctgggacgtggtggctttggagttgtttttgaagctaaaaacaaagtagatgactgcaattatgctatcaagaggatccgtctccccaatagggaattggctcgggaaaaggtaatgcgagaagttaaagccttagccaagcttgaacacccgggcattgttagatatttcaatgcctggctcgaagcaccaccagagaagtggcaagaaaagatggatgaaatttggctgaaagatgaaagcacagactggccactcagctctcctagcccaatggatgcaccatcagttaaaatacgcagaatggatcctttcgctacaaaagaacatattgaaatcatagctccttcaccacaaagaagcaggtctttttcagtagggatttcctgtgaccagacaagttcatctgagagccagttctcaccactggaattctcaggaatggaccatgaggacatcagtgagtcagtggatgcagcatacaacctccaggacagttgccttacagactgtgatgtggaagatgggactatggatggcaatgatgaggggcactcctttgaactttgtccttctgaagcttctccttatgtaaggtcaagggagagaacctcctcttcaatagtatttgaagattctggctgtgataatgcttccagtaaagaagagccgaaaactaatcgattgcatattggcaaccattgtgctaataaactaactgctttcaagcccaccagtagcaaatcttcttctgaagctacattgtctatttctcctccaagaccaaccactttaagtttagatctcactaaaaacaccacagaaaaactccagcccagttcaccaaaggtgtatctttacattcaaatgcagctgtgcagaaaagaaaacctcaaagactggatgaatggacgatgtaccatagaggagagagagaggagcgtgtgtctgcacatcttcctgcagatcgcagaggcagtggagtttcttcacagtaaaggactgatgcacagggacctcaagccatccaacatattctttacaatggatgatgtggtcaaggttggagactttgggttagtgactgcaatggaccaggatgaggaagagcagacggttctgaccccaatgccagcttatgccagacacacaggacaagtagggaccaaactgtatatgagcccagagcagattcatggaaacagctattctcataaagtggacatcttttctttaggcctgattctatttgaattgctgtatccattcagcactcagatggagagagtcaggaccttaactgatgtaagaaatctcaaatttccaccattatttactcagaaatatccttgtgagtacgtgatggttcaagacatgctctctccatcccccatggaacgacctgaagctataaacatcattgaaaatgctgtatttgaggacttggactttccaggaaaaacagtgctcagacagaggtctcgctccttgagttcatcgggaacaaaacattcaagacagtccaacaactcccatagccctttgccaagcaattagccttaagttgtgctagcaaccctaataggtgatgcagataatagcctacttcttagaatatgcctgtccaaaattgcagacttgaaaagtttgttcttcgctcaatttttttgtggactactttttttatatcaaatttaagctggatttgggggcataacctaatttgagccaactcctgagttttgctatacttaaggaaagggctatctttgttctttgttagtctcttgaaactggctgctggccaagctttatagccctcaccatttgcctaaggaggtagcagcaatccctaatatatatatatagtgagaactaaaatggatatatttttataatgcagaagaaggaaagtccccctgtgtggtaactgtattgttctagaaatatgctttctagagatatgatgattttgaaactgatttctagaaaaagctgactccatttttgtccctggcgggtaaattaggaatctgcactattttggaggacaagtagcacaaactgtataacggtttatgtccgtagttttatagtcctatttgtagcattcaatagctttattccttagatggttctagggtgggtttacagctttttgtacttttacctccaataaagggaaaatgaagctttttatgtaaattggttgaaaggtctagttttgggaggaaaaaagccgtagtaagaaatggatcatatatattacaactaacttcttcaactatggactttttaagcctaatgaaatcttaagtgtcttatatgtaatcctgtaggttggtacttcccccaaactgattataggtaacagtttaatcatctcacttgctaacatgtttttatttttcactgtaaatatgtttatgttttatttataaaaattctgaaatcaatccatttgggttggtggtgtacagaacacacttaagtgtgttaacttgtgacttctttcaagtctaaatgatttaataaaactttttttaaattaaaaaaaaaaaaaaaaaaa

SEQ ID NO: 41 - protein, 1116 aa, Homo sapiens
  ref <308>: DATABASE ACCESSION NUMBER: NCBI / NP_004827.4
  ref <309>: DATABASE ENTRY DATE: 2011-10-16
  ref <313>: RELEVANT RESIDUES IN SEQ ID NO: (1)..(1116)
MERAISPGLLVRALLLLLLLLGLAARTVAAGRARGLPAPTAEAAFGLGAAAAPTSATRVPAAGAVAAAEVTVEDAEALPAAAGEQEPRGPEPDDETELRPRGRSLVIISTLDGRIAALDPENHGKKQWDLDVGSGSLVSSSLSKPEVFGNKMIIPSLDGALFQWDQDRESMETVPFTVESLLESSYKFGDDVVLVGGKSLTTYGLSAYSGKVRYICSALGCRQWDSDEMEQEEDILLLQRTQKTVRAVGPRSGNEKWNFSVGHFELRYIPDMETRAGFIESTFKPNENTEESKIISDVEEQEAAIMDIVIKVSVADWKVMAFSKKGGHLEWEYQFCTPIASAWLLKDGKVIPISLFDDTSYTSNDDVLEDEEDIVEAARGATENSVYLGMYRGQLYLQSSVRISEKFPSSPKALESVTNENAIIPLPTIKWKPLIHSPSRTPVLVGSDEFDKCLSNDKFSHEEYSNGALSILQYPYDNGYYLPYYKRERNKRSTQITVRFLDNPHYNKNIRKKDPVLLLHWWKEIVATILFCIIATTFIVRRLFHPHPHRQRKESETQCQTENKYDSVSGEANDSSWNDIKNSGYISRYLTDFEPIQCLGRGGFGVVFEAKNKVDDCNYAIKRIRLPNRELAREKVMREVKALAKLEHPGIVRYFNAWLEAPPEKWQEKMDEIWLKDESTDWPLSSPSPMDAPSVKIRRMDPFATKEHIEIIAPSPQRSRSFSVGISCDQTSSSESQFSPLEFSGMDHEDISESVDAAYNLQDSCLTDCDVEDGTMDGNDEGHSFELCPSEASPYVRSRERTSSSIVFEDSGCDNASSKEEPKTNRLHIGNHCANKLTAFKPTSSKSSSEATLSISPPRPTTLSLDLTKNTTEKLQPSSPKVYLYIQMQLCRKENLKDWMNGRCTIEERERSVCLHIFLQIAEAVEFLHSKGLMHRDLKPSNIFFTMDDVVKVGDFGLVTAMDQDEEEQTVLTPMPAYARHTGQVGTKLYMSPEQIHGNSYSHKVDIFSLGLILFELLYPFSTQMERVRTLTDVRNLKFPPLFTQKYPCEYVMVQDMLSPSPMERPEAINIIENAVFEDLDFPGKTVLRQRSRSLSSSGTKHSRQSNNSHSPLPSN

SEQ ID NO: 42 - protein, 192 aa, Homo sapiens
  ref <308>: DATABASE ACCESSION NUMBER: NCBI / NP_001181982.1
  ref <309>: DATABASE ENTRY DATE: 2011-10-23
  ref <313>: RELEVANT RESIDUES IN SEQ ID NO: (1)..(192)
MELVPATPHYPADVLFQTDPTAEMAAESLPFSFGTLSSWELEAWYEDLQEVLSSDENGGTYVSPPGNEEEESKIFTTLDPASLAWLTEEEPEPAEVTSTSQSPHSPDSSQSSLAQEEEEEDQGRTRKRKQSGHSPARAGKQRMKEKEQENERKVAQLAEENERLKQEIERLTREVEATRRALIDRMVNLHQA

SEQ ID NO: 43 - protein, 192 aa, Homo sapiens; identical to SEQ ID NO: 42
  ref <308>: DATABASE ACCESSION NUMBER: NCBI / NP_001181983.1
  ref <309>: DATABASE ENTRY DATE: 2011-10-23
  ref <313>: RELEVANT RESIDUES IN SEQ ID NO: (1)..(192)

SEQ ID NO: 44 - protein, 192 aa, Homo sapiens; identical to SEQ ID NO: 42
  ref <308>: DATABASE ACCESSION NUMBER: NCBI / NP_001181984.1
  ref <309>: DATABASE ENTRY DATE: 2011-10-23
  ref <313>: RELEVANT RESIDUES IN SEQ ID NO: (1)..(192)

SEQ ID NO: 45 - protein, 192 aa, Homo sapiens; identical to SEQ ID NO: 42
  ref <308>: DATABASE ACCESSION NUMBER: NCBI / NP_001181985.1,
  ref <309>: DATABASE ENTRY DATE: 2011-10-23
  ref <313>: RELEVANT RESIDUES IN SEQ ID NO: (1)..(192)

SEQ ID NO: 46 - protein, 169 aa, Homo sapiens
  ref <308>: DATABASE ACCESSION NUMBER: NCBI / NP_001181986.1
  ref <309>: DATABASE ENTRY DATE: 2011-10-23
  ref <313>: RELEVANT RESIDUES IN SEQ ID NO: (1)..(169)
MAAESLPFSFGTLSSWELEAWYEDLQEVLSSDENGGTYVSPPGNEEEESKIFTTLDPASLAWLTEEEPEPAEVTSTSQSPHSPDSSQSSLAQEEEEEDQGRTRKRKQSGHSPARAGKQRMKEKEQENERKVAQLAEENERLKQEIERLTREVEATRRALIDRMVNLHQA

SEQ ID NO: 47 - protein, 169 aa, Homo sapiens; identical to SEQ ID NO: 46
  ref <308>: DATABASE ACCESSION NUMBER: NCBI / NP_004074.2
  ref <309>: DATABASE ENTRY DATE: 2011-10-23
  ref <313>: RELEVANT RESIDUES IN SEQ ID NO: (1)..(169)

SEQ ID NO: 48 - DNA, 1081 bp, Homo sapiens
  ref <308>: DATABASE ACCESSION NUMBER: NCBI / NM_001195053.1
  ref <309>: DATABASE ENTRY DATE: 2011-10-23
  ref <313>: RELEVANT RESIDUES IN SEQ ID NO: (1)..(1081)
gaggtcagagacttaagtctaaggcactgagcgtatcatgttaaagatgagcgggtggcagcgacagagccaaaatcagagctggaacctgaggagagagtgttcaagaaggaagtgtatcttcatacatcaccacacctgaaagcagcaccaaagcagccataaacaatatgtaaataaacagatgtggctgtattccagtacaactttacctacaaaaacaggcatcagaccagcttgccaacttgtggcatagactgtttgctacatggagcttgttccagccactccccattatcctgcagatgtgcttttccagactgatccaactgcagagatggcagctgagtcattgcctttctccttcgggacactgtccagctgggagctggaagcctggtatgaggacctgcaagaggtcctgtcttcagatgaaaatgggggtacctatgtttcacctcctggaaatgaagaggaagaatcaaaaatcttcaccactcttgaccctgcttctctggcttggctgactgaggaggagccagaaccagcagaggtcacaagcacctcccagagccctcactctccagattccagtcagagctccctggctcaggaggaagaggaggaagaccaagggagaaccaggaaacggaaacagagtggtcattccccagcccgggctggaaagcagcgcatgaaggagaaagaacaggagaatgaaaggaaagtggcacagctagctgaagagaatgaacggctcaagcaggaaatcgagcgcctgaccagggaagtagaggcgactcgccgagctctgattgaccgaatggtgaatctgcaccaagcatgaacaattgggagcatcagtcccccacttgggccacactacccacctttcccagaagtggctactgactaccctctcactagtgccaatgatgtgaccctcaatcccacatacgcagggggaaggcttggagtagacaaaaggaaaggtctcagcttgtatatagagattgtacatttatttattactgtccctatctattaaagtgactttctatgagccaaaaaaaaaaaaaaa

SEQ ID NO: 49 - DNA, 1028 bp, Homo sapiens
  ref <308>: DATABASE ACCESSION NUMBER: NCBI / NM_001195054.1
  ref <309>: DATABASE ENTRY DATE: 2011-10-23
  ref <313>: RELEVANT RESIDUES IN SEQ ID NO: (1)..(1028)
gaggtcagagacttaagtctaaggcactgagcgtatcatgttaaagatgagcgggtggcagcgacagagccaaaatcagagctggaacctgaggagagagtgttcaagaaggaagtgtatcttcatacatcaccacacctgaaagcagtacaactttacctacaaaaacaggcatcagaccagcttgccaacttgtggcatagactgtttgctacatggagcttgttccagccactccccattatcctgcagatgtgcttttccagactgatccaactgcagagatggcagctgagtcattgcctttctccttcgggacactgtccagctgggagctggaagcctggtatgaggacctgcaagaggtcctgtcttcagatgaaaatgggggtacctatgtttcacctcctggaaatgaagaggaagaatcaaaaatcttcaccactcttgaccctgcttctctggcttggctgactgaggaggagccagaaccagcagaggtcacaagcacctcccagagccctcactctccagattccagtcagagctccctggctcaggaggaagaggaggaagaccaagggagaaccaggaaacggaaacagagtggtcattccccagcccgggctggaaagcagcgcatgaaggagaaagaacaggagaatgaaaggaaagtggcacagctagctgaagagaatgaacggctcaagcaggaaatcgagcgcctgaccagggaagtagaggcgactcgccgagctctgattgaccgaatggtgaatctgcaccaagcatgaacaattgggagcatcagtcccccacttgggccacactacccacctttcccagaagtggctactgactaccctctcactagtgccaatgatgtgaccctcaatcccacatacgcagggggaaggcttggagtagacaaaaggaaaggtctcagcttgtatatagagattgtacatttatttattactgtccctatctattaaagtgactttctatgagccaaaaaaaaaaaaaaa

SEQ ID NO: 50 - DNA, 1005 bp, Homo sapiens
  ref <308>: DATABASE ACCESSION NUMBER: NCBI / NM_001195055.1
  ref <309>: DATABASE ENTRY DATE: 2011-10-23
  ref <313>: RELEVANT RESIDUES IN SEQ ID NO: (1)..(1005)
gaggtcagagacttaagtctaaggcactgagcgtatcatgttaaagatgagcgggtggcagcgacagagccaaaatcagagctggaacctgaggagagagtgttcaagaaggaagtgtatcttcatacatcaccacacctgaaagcaggcatcagaccagcttgccaacttgtggcatagactgtttgctacatggagcttgttccagccactccccattatcctgcagatgtgcttttccagactgatccaactgcagagatggcagctgagtcattgcctttctccttcgggacactgtccagctgggagctggaagcctggtatgaggacctgcaagaggtcctgtcttcagatgaaaatgggggtacctatgtttcacctcctggaaatgaagaggaagaatcaaaaatcttcaccactcttgaccctgcttctctggcttggctgactgaggaggagccagaaccagcagaggtcacaagcacctcccagagccctcactctccagattccagtcagagctccctggctcaggaggaagaggaggaagaccaagggagaaccaggaaacggaaacagagtggtcattccccagcccgggctggaaagcagcgcatgaaggagaaagaacaggagaatgaaaggaaagtggcacagctagctgaagagaatgaacggctcaagcaggaaatcgagcgcctgaccagggaagtagaggcgactcgccgagctctgattgaccgaatggtgaatctgcaccaagcatgaacaattgggagcatcagtcccccacttgggccacactacccacctttcccagaagtggctactgactaccctctcactagtgccaatgatgtgaccctcaatcccacatacgcagggggaaggcttggagtagacaaaaggaaaggtctcagcttgtatatagagattgtacatttatttattactgtccctatctattaaagtgactttctatgagccaaaaaaaaaaaaaaa

SEQ ID NO: 51 - DNA, 1191 bp, Homo sapiens
  ref <308>: DATABASE ACCESSION NUMBER: NCBI / NM_001195056.1
  ref <309>: DATABASE ENTRY DATE: 2011-10-23
  ref <313>: RELEVANT RESIDUES IN SEQ ID NO: (1)..(1191)
gaggtcagagacttaagtctaaggcactgagcgtatcatgttaaagatgagcgggtggcagcgacagagccaaaatcagagctggaacctgaggagagagtgttcaagaaggaagtgtatcttcatacatcaccacacctgaaagcaggtaaacttaacctacccttttccaaaaattttaaacggcaggacagtaaatattttagatgttaaaagtcctatagtctctagcgtgactcttcatctctgccactgtagcaccaaagcagccataaacaatatgtaaataaacagatgtggctgtattccagtacaactttacctacaaaaacaggcatcagaccagcttgccaacttgtggcatagactgtttgctacatggagcttgttccagccactccccattatcctgcagatgtgcttttccagactgatccaactgcagagatggcagctgagtcattgcctttctccttcgggacactgtccagctgggagctggaagcctggtatgaggacctgcaagaggtcctgtcttcagatgaaaatgggggtacctatgtttcacctcctggaaatgaagaggaagaatcaaaaatcttcaccactcttgaccctgcttctctggcttggctgactgaggaggagccagaaccagcagaggtcacaagcacctcccagagccctcactctccagattccagtcagagctccctggctcaggaggaagaggaggaagaccaagggagaaccaggaaacggaaacagagtggtcattccccagcccgggctggaaagcagcgcatgaaggagaaagaacaggagaatgaaaggaaagtggcacagctagctgaagagaatgaacggctcaagcaggaaatcgagcgcctgaccagggaagtagaggcgactcgccgagctctgattgaccgaatggtgaatctgcaccaagcatgaacaattgggagcatcagtcccccacttgggccacactacccacctttcccagaagtggctactgactaccctctcactagtgccaatgatgtgaccctcaatcccacatacgcagggggaaggcttggagtagacaaaaggaaaggtctcagcttgtatatagagattgtacatttatttattactgtccctatctattaaagtgactttctatgagccaaaaaaaaaaaaaaa

SEQ ID NO: 52 - DNA, 910 bp, Homo sapiens
  ref <308>: DATABASE ACCESSION NUMBER: NCBI / NM_001195057.1
  ref <309>: DATABASE ENTRY DATE: 2011-10-23
  ref <313>: RELEVANT RESIDUES IN SEQ ID NO: (1)..(910)
gaggtcagagacttaagtctaaggcactgagcgtatcatgttaaagatgagcgggtggcagcgacagagccaaaatcagagctggaacctgaggagagagtgttcaagaaggaagtgtatcttcatacatcaccacacctgaaagcagactgatccaactgcagagatggcagctgagtcattgcctttctccttcgggacactgtccagctgggagctggaagcctggtatgaggacctgcaagaggtcctgtcttcagatgaaaatgggggtacctatgtttcacctcctggaaatgaagaggaagaatcaaaaatcttcaccactcttgaccctgcttctctggcttggctgactgaggaggagccagaaccagcagaggtcacaagcacctcccagagccctcactctccagattccagtcagagctccctggctcaggaggaagaggaggaagaccaagggagaaccaggaaacggaaacagagtggtcattccccagcccgggctggaaagcagcgcatgaaggagaaagaacaggagaatgaaaggaaagtggcacagctagctgaagagaatgaacggctcaagcaggaaatcgagcgcctgaccagggaagtagaggcgactcgccgagctctgattgaccgaatggtgaatctgcaccaagcatgaacaattgggagcatcagtcccccacttgggccacactacccacctttcccagaagtggctactgactaccctctcactagtgccaatgatgtgaccctcaatcccacatacgcagggggaaggcttggagtagacaaaaggaaaggtctcagcttgtatatagagattgtacatttatttattactgtccctatctattaaagtgactttctatgagccaaaaaaaaaaaaaaa

SEQ ID NO: 53 - DNA, 924 bp, Homo sapiens
  ref <308>: DATABASE ACCESSION NUMBER: NCBI / NM_004083.5
  ref <309>: DATABASE ENTRY DATE: 2011-10-23
  ref <313>: RELEVANT RESIDUES IN SEQ ID NO: (1)..(924)
gaggtcagagacttaagtctaaggcactgagcgtatcatgttaaagatgagcgggtggcagcgacagagccaaaatcagagctggaacctgaggagagagtgttcaagaaggaagtgtatcttcatacatcaccacacctgaaagcagatgtgcttttccagactgatccaactgcagagatggcagctgagtcattgcctttctccttcgggacactgtccagctgggagctggaagcctggtatgaggacctgcaagaggtcctgtcttcagatgaaaatgggggtacctatgtttcacctcctggaaatgaagaggaagaatcaaaaatcttcaccactcttgaccctgcttctctggcttggctgactgaggaggagccagaaccagcagaggtcacaagcacctcccagagccctcactctccagattccagtcagagctccctggctcaggaggaagaggaggaagaccaagggagaaccaggaaacggaaacagagtggtcattccccagcccgggctggaaagcagcgcatgaaggagaaagaacaggagaatgaaaggaaagtggcacagctagctgaagagaatgaacggctcaagcaggaaatcgagcgcctgaccagggaagtagaggcgactcgccgagctctgattgaccgaatggtgaatctgcaccaagcatgaacaattgggagcatcagtcccccacttgggccacactacccacctttcccagaagtggctactgactaccctctcactagtgccaatgatgtgaccctcaatcccacatacgcagggggaaggcttggagtagacaaaaggaaaggtctcagcttgtatatagagattgtacatttatttattactgtccctatctattaaagtgactttctatgagccaaaaaaaaaaaaaaa

SEQ ID NO: 54 - protein, 592 aa, Homo sapiens
  ref <308>: DATABASE ACCESSION NUMBER: NCBI / NP_001019820.1
  ref <309>: DATABASE ENTRY DATE: 2011-10-23
  ref <313>: RELEVANT RESIDUES IN SEQ ID NO: (1)..(592)
MEGKWLLCMLLVLGTAIVEAHDGHDDDVIDIEDDLDDVIEEVEDSKPDTTAPPSSPKVTYKAPVPTGEVYFADSFDRGTLSGWILSKAKKDDTDDEIAKYDGKWEVEEMKESKLPGDKGLVLMSRAKHHAISAKLNKPFLFDTKPLIVQYEVNFQNGIECGGAYVKLLSKTPELNLDQFHDKTPYTIMFGPDKCGEDYKLHFIFRHKNPKTGIYEEKHAKRPDADLKTYFTDKKTHLYTLILNPDNSFEILVDQSVVNSGNLLNDMTPPVNPSREIEDPEDRKPEDWDERPKIPDPEAVKPDDWDEDAPAKIPDEEATKPEGWLDDEPEYVPDPDAEKPEDWDEDMDGEWEAPQIANPRCESAPGCGVWQRPVIDNPNYKGKWKPPMIDNPSYQGIWKPRKIPNPDFFEDLEPFRMTPFSAIGLELWSMTSDIFFDNFIICADRRIVDDWANDGWGLKKAADGAAEPGVVGQMIEAAEERPWLWVVYILTVALPVFLVILFCCSGKKQTSGMEYKKTDAPQPDVKEEEEEKEEEKDKGDEEEEGEEKLEEKQKSDAEEDGGTVSQEEEDRKPKAEEDEILNRSPRNRKPRRE

SEQ ID NO: 55 - protein, 592 aa, Homo sapiens; identical to SEQ ID NO: 54
  ref <308>: DATABASE ACCESSION NUMBER: NCBI / NP_001737.1
  ref <309>: DATABASE ENTRY DATE: 2011-10-23
  ref <313>: RELEVANT RESIDUES IN SEQ ID NO: (1)..(592)

SEQ ID NO: 56 - DNA, 4900 bp, Homo sapiens
  ref <308>: DATABASE ACCESSION NUMBER: NCBI / NM_001024649.1
  ref <309>: DATABASE ENTRY DATE: 2011-10-23
  ref <313>: RELEVANT RESIDUES IN SEQ ID NO: (1)..(4900)
agggccgggcttcgtgcggtggggctcgctcgcgcggcagcggtggccgaggcctcttggttctgcggcacgtgacggtcgggccgcctccgcctctctctttactgcggcgcggggcaagatcatggaagggaagtggttgctgtgtatgttactggtgcttggaactgctattgttgaggctcatgatggacatgatgatgatgtgattgatattgaggatgaccttgacgatgtcattgaagaggtagaagactcaaaaccagataccactgctcctccttcatctcccaaggttacttacaaagctccagttccaacaggggaagtatattttgctgattcttttgacagaggaactctgtcagggtggattttatccaaagccaagaaagacgataccgatgatgaaattgccaaatatgatggaaagtgggaggtagaggaaatgaaggagtcaaagcttccaggtgataaaggacttgtgttgatgtctcgggccaagcatcatgccatctctgctaaactgaacaagcccttcctgtttgacaccaagcctctcattgttcagtatgaggttaatttccaaaatggaatagaatgtggtggtgcctatgtgaaactgctttctaaaacaccagaactcaacctggatcagttccatgacaagaccccttatacgattatgtttggtccagataaatgtggagaggactataaactgcacttcatcttccgacacaaaaaccccaaaacgggtatctatgaagaaaaacatgctaagaggccagatgcagatctgaagacctattttactgataagaaaacacatctttacacactaatcttgaatccagataatagttttgaaatactggttgaccaatctgtggtgaatagtggaaatctgctcaatgacatgactcctcctgtaaatccttcacgtgaaattgaggacccagaagaccggaagcccgaggattgggatgaaagaccaaaaatcccagatccagaagctgtcaagccagatgactgggatgaagatgcccctgctaagattccagatgaagaggccacaaaacccgaaggctggttagatgatgagcctgagtacgtacctgatccagacgcagagaaacctgaggattgggatgaagacatggatggagaatgggaggctcctcagattgccaaccctagatgtgagtcagctcctggatgtggtgtctggcagcgacctgtgattgacaaccccaattataaaggcaaatggaagcctcctatgattgacaatcccagttaccagggaatctggaaacccaggaaaataccaaatccagatttctttgaagatctggaacctttcagaatgactccttttagtgctattggtttggagctgtggtccatgacctctgacattttttttgacaactttatcatttgtgctgatcgaagaatagttgatgattgggccaatgatggatggggcctgaagaaagctgctgatggggctgctgagccaggcgttgtggggcagatgatcgaggcagctgaagagcgcccgtggctgtgggtagtctatattctaactgtagcccttcctgtgttcctggttatcctcttctgctgttctggaaagaaacagaccagtggtatggagtataagaaaactgatgcacctcaaccggatgtgaaggaagaggaagaagagaaggaagaggaaaaggacaagggagatgaggaggaggaaggagaagagaaacttgaagagaaacagaaaagtgatgctgaagaagatggtggcactgtcagtcaagaggaggaagacagaaaacctaaagcagaggaggatgaaattttgaacagatcaccaagaaacagaaagccacgaagagagtgaaacaatcttaagagcttgatctgtgatttcttctccctcctcccctgcaagagtggtcctaggagaggacctggcacaccttaggttgacattcagaaaacttcaagacatcaccatcagcaggctccagttgaacactagtctgtgtaactttaaacatctagcagtaaatacttgcagttgtgatataaaggaccctgtttctgtagaaaagaaaacatttaacataatggttgtgaaatgtaacatgaagcaaactaacttttttttttttaacatctttgtttttaaaatagaatgatagaactttgccagtctttaagatcttggcttaatttaatgtattaatctgtttgtgcaaacataataccaccatttaaaaatgttagggagatgagttgcagtttttataatagattttttttaaagtttggtattgtaaaacattcacacctctgtccctcaaaattgataattacgtttaaagtgcagtcatttgtggttagaatcttgttttgtttgcttccattattgagttcctcctaaggaaattgaggagagggactgaatagaagcccaaattcatataaaagttgcgtttaagttgtattaaaaatagatatataagaaaaaattctttcacttgatgtttgttagaccagaaagtgtgtgtgttctgtagctcagttcccagacagctttttaggtagtggaggaggtggcttcatgtggcacttgggcatttatattccacttgggagggtcaggctgtggccttctggagcaggtggcttgttaaggaatgctagcagggcatggcacgtgagctccggaatagatgtcttcatcacttcttccactgtgtgttgacactgttttccttacctatttcctcagatccccagctttctcctctgctatgcattttcttcacagtgcagcttgcagtccgttgctgaaaatgattataagccctgcataatgttaagctttattgtgattacgtgtatgtttcttctttcttttaagcagacccatacctttccagggtcaaagtacagaatagaatacattgatacaaagtacagaaaaatactttgatttttatccatttcttttactctgtgtaaagacttgagaagtctaattcacaggcaaaccaatacagaattgactgcagttgaacagactagaagtatttgtgggaggagtgacatgaagcatgagttatctgattttttttgtagctgctatatattttaagccttcatttgcaattcatgtaacagttgtgtcataaattacacaataaagcagtcctgttcaaatttttttttaacgtggcttgtagaatttttaaaaaagtgatcttaggtttgttttttcatgcgggatgcagatgggtgctatcagagcctctcccacaccactatagtgtaataatgttattattactctacactgaaacgtattcagagttagatattattttagcttcagttgttctttagaggctttcaaatgtaccgatgatactgtttcttgcactgaatatataaacactccacagtgtttatattgggaagatattgggaaggaaatatatttgtaaaagatgaaggctgtatctatttttttttctttttaaagtttgttcacttaaattcttttgaggatgggatgtatttttcttgctgttcagtgctttttccttttcatctgttgttctgtggtcacagtgaccttagctacatagcagactttcccaaatgtattgattacaaataaacagttgttacttagcaagacctgaaaatatgtctgcaggtttctccttgaagcaaatgtgtgggatcattgcatttccagaaatctgcctccttcaccctccgttgacagtatatgtcatgcctcactttcttctagctgagctttaaatcattagagcttaaattgtcagatcgttcattgcctttccagggttatttagtaaagtttgttgaaaacaaaaacgccttttcttggttcttttttcagttattttgaaggtccagcatcctgattaaatgtctgacacattaatgaatgaccagcagcagctttcagctcttaaaaagacacttatattttgattttacatgctggttacctgttccattgttgtcaaatgcccactctccatcagatgtgttcctccattttcttatccacaaagtactcctcacttttcaatttgtcatgttactaaatggtgttacattaaagccctgtgttaagtgtctgcttttgactgaatttcttcatagtaaccttcagtgtgtgtgtgtgactatgttcaattagtgggttgatcttcgtataattggccactatgtgagagttcactactaggcagaaactattatggacagtgaaataatgacttttatctcaccacgtgagtttgatgcagtcttttctgtctagcccttgcctcttcctgcccatgtgattgcggtgcagtagtttctgttgtataatagtgtggacagcagctcagaaaaggagggaatgctactgataatttgtagataatattctttaagacttaggggaaccattgaactttgaaatttttattagaaaattatttgttcagaatcagactccattattttacatatacttaaatactttaggggtatttttgaaagttagcccagttttttatgtgctattaaatttttagattacatactaaagaaaagtatgtacacagaatgtagtgctcctagttactattttttctattaagaaatagtttagttctggtgtaaaatttgtttgaatgctaaaaaaaaaaaagcaggactgcattatggcacttttgccttggtggcccactttctccatttaaaacattaggatttgtatttttctgctgcgtttgtatgaagacatatttgattgttgttttctcttgattttaaaataaaacctcatgagccctagtaaaaa

SEQ ID NO: 57 - DNA, 4953 bp, Homo sapiens
  ref <308>: DATABASE ACCESSION NUMBER: NCBI / NM_001746.3
  ref <309>: DATABASE ENTRY DATE: 2011-10-23
  ref <313>: RELEVANT RESIDUES IN SEQ ID NO: (1)..(4953)
agggccgggcttcgtgcggtggggctcgctcgcgcggcagcggtggccgaggcctcttggttctgcggcacgtgacggtcgggccgcctccgcctctctctttactgcggcgcggggcaaggtgtgcgggcgggaaggggcacgggcacccccgcggtccccgggaggctagagatcatggaagggaagtggttgctgtgtatgttactggtgcttggaactgctattgttgaggctcatgatggacatgatgatgatgtgattgatattgaggatgaccttgacgatgtcattgaagaggtagaagactcaaaaccagataccactgctcctccttcatctcccaaggttacttacaaagctccagttccaacaggggaagtatattttgctgattcttttgacagaggaactctgtcagggtggattttatccaaagccaagaaagacgataccgatgatgaaattgccaaatatgatggaaagtgggaggtagaggaaatgaaggagtcaaagcttccaggtgataaaggacttgtgttgatgtctcgggccaagcatcatgccatctctgctaaactgaacaagcccttcctgtttgacaccaagcctctcattgttcagtatgaggttaatttccaaaatggaatagaatgtggtggtgcctatgtgaaactgctttctaaaacaccagaactcaacctggatcagttccatgacaagaccccttatacgattatgtttggtccagataaatgtggagaggactataaactgcacttcatcttccgacacaaaaaccccaaaacgggtatctatgaagaaaaacatgctaagaggccagatgcagatctgaagacctattttactgataagaaaacacatctttacacactaatcttgaatccagataatagttttgaaatactggttgaccaatctgtggtgaatagtggaaatctgctcaatgacatgactcctcctgtaaatccttcacgtgaaattgaggacccagaagaccggaagcccgaggattgggatgaaagaccaaaaatcccagatccagaagctgtcaagccagatgactgggatgaagatgcccctgctaagattccagatgaagaggccacaaaacccgaaggctggttagatgatgagcctgagtacgtacctgatccagacgcagagaaacctgaggattgggatgaagacatggatggagaatgggaggctcctcagattgccaaccctagatgtgagtcagctcctggatgtggtgtctggcagcgacctgtgattgacaaccccaattataaaggcaaatggaagcctcctatgattgacaatcccagttaccagggaatctggaaacccaggaaaataccaaatccagatttctttgaagatctggaacctttcagaatgactccttttagtgctattggtttggagctgtggtccatgacctctgacattttttttgacaactttatcatttgtgctgatcgaagaatagttgatgattgggccaatgatggatggggcctgaagaaagctgctgatggggctgctgagccaggcgttgtggggcagatgatcgaggcagctgaagagcgcccgtggctgtgggtagtctatattctaactgtagcccttcctgtgttcctggttatcctcttctgctgttctggaaagaaacagaccagtggtatggagtataagaaaactgatgcacctcaaccggatgtgaaggaagaggaagaagagaaggaagaggaaaaggacaagggagatgaggaggaggaaggagaagagaaacttgaagagaaacagaaaagtgatgctgaagaagatggtggcactgtcagtcaagaggaggaagacagaaaacctaaagcagaggaggatgaaattttgaacagatcaccaagaaacagaaagccacgaagagagtgaaacaatcttaagagcttgatctgtgatttcttctccctcctcccctgcaagagtggtcctaggagaggacctggcacaccttaggttgacattcagaaaacttcaagacatcaccatcagcaggctccagttgaacactagtctgtgtaactttaaacatctagcagtaaatacttgcagttgtgatataaaggaccctgtttctgtagaaaagaaaacatttaacataatggttgtgaaatgtaacatgaagcaaactaacttttttttttttaacatctttgtttttaaaatagaatgatagaactttgccagtctttaagatcttggcttaatttaatgtattaatctgtttgtgcaaacataataccaccatttaaaaatgttagggagatgagttgcagtttttataatagattttttttaaagtttggtattgtaaaacattcacacctctgtccctcaaaattgataattacgtttaaagtgcagtcatttgtggttagaatcttgttttgtttgcttccattattgagttcctcctaaggaaattgaggagagggactgaatagaagcccaaattcatataaaagttgcgtttaagttgtattaaaaatagatatataagaaaaaattctttcacttgatgtttgttagaccagaaagtgtgtgtgttctgtagctcagttcccagacagctttttaggtagtggaggaggtggcttcatgtggcacttgggcatttatattccacttgggagggtcaggctgtggccttctggagcaggtggcttgttaaggaatgctagcagggcatggcacgtgagctccggaatagatgtcttcatcacttcttccactgtgtgttgacactgttttccttacctatttcctcagatccccagctttctcctctgctatgcattttcttcacagtgcagcttgcagtccgttgctgaaaatgattataagccctgcataatgttaagctttattgtgattacgtgtatgtttcttctttcttttaagcagacccatacctttccagggtcaaagtacagaatagaatacattgatacaaagtacagaaaaatactttgatttttatccatttcttttactctgtgtaaagacttgagaagtctaattcacaggcaaaccaatacagaattgactgcagttgaacagactagaagtatttgtgggaggagtgacatgaagcatgagttatctgattttttttgtagctgctatatattttaagccttcatttgcaattcatgtaacagttgtgtcataaattacacaataaagcagtcctgttcaaatttttttttaacgtggcttgtagaatttttaaaaaagtgatcttaggtttgttttttcatgcgggatgcagatgggtgctatcagagcctctcccacaccactatagtgtaataatgttattattactctacactgaaacgtattcagagttagatattattttagcttcagttgttctttagaggctttcaaatgtaccgatgatactgtttcttgcactgaatatataaacactccacagtgtttatattgggaagatattgggaaggaaatatatttgtaaaagatgaaggctgtatctatttttttttctttttaaagtttgttcacttaaattcttttgaggatgggatgtatttttcttgctgttcagtgctttttccttttcatctgttgttctgtggtcacagtgaccttagctacatagcagactttcccaaatgtattgattacaaataaacagttgttacttagcaagacctgaaaatatgtctgcaggtttctccttgaagcaaatgtgtgggatcattgcatttccagaaatctgcctccttcaccctccgttgacagtatatgtcatgcctcactttcttctagctgagctttaaatcattagagcttaaattgtcagatcgttcattgcctttccagggttatttagtaaagtttgttgaaaacaaaaacgccttttcttggttcttttttcagttattttgaaggtccagcatcctgattaaatgtctgacacattaatgaatgaccagcagcagctttcagctcttaaaaagacacttatattttgattttacatgctggttacctgttccattgttgtcaaatgcccactctccatcagatgtgttcctccattttcttatccacaaagtactcctcacttttcaatttgtcatgttactaaatggtgttacattaaagccctgtgttaagtgtctgcttttgactgaatttcttcatagtaaccttcagtgtgtgtgtgtgactatgttcaattagtgggttgatcttcgtataattggccactatgtgagagttcactactaggcagaaactattatggacagtgaaataatgacttttatctcaccacgtgagtttgatgcagtcttttctgtctagcccttgcctcttcctgcccatgtgattgcggtgcagtagtttctgttgtataatagtgtggacagcagctcagaaaaggagggaatgctactgataatttgtagataatattctttaagacttaggggaaccattgaactttgaaatttttattagaaaattatttgttcagaatcagactccattattttacatatacttaaatactttaggggtatttttgaaagttagcccagttttttatgtgctattaaatttttagattacatactaaagaaaagtatgtacacagaatgtagtgctcctagttactattttttctattaagaaatagtttagttctggtgtaaaatttgtttgaatgctaaaaaaaaaaaagcaggactgcattatggcacttttgccttggtggcccactttctccatttaaaacattaggatttgtatttttctgctgcgtttgtatgaagacatatttgattgttgttttctcttgattttaaaataaaacctcatgagccctagtaaaaa

SEQ ID NO: 58 - protein, 930 aa, Homo sapiens
  ref <308>: DATABASE ACCESSION NUMBER: NCBI / NP_060362.3
  ref <309>: DATABASE ENTRY DATE: 2011-09-17
  ref <313>: RELEVANT RESIDUES IN SEQ ID NO: (1)..(930)
MCSGSGRRRSSLSPTMRPGTGAERGGLMMGHPGMHYAPMGMHPMGQRANMPPVPHGMMPQMMPPMGGPPMGQMPGMMSSVMPGMMMSHMSQASMQPALPPGVNSMDVAAGTASGAKSMWTEHKSPDGRTYYYNTETKQSTWEKPDDLKTPAEQLLSKCPWKEYKSDSGKPYYYNSQTKESRWAKPKELEDLEGYQNTIVAGSLITKSNLHAMIKAEESSKQEECTTTSTAPVPTTEIPTTMSTMAAAEAAAAVVAAAAAAAAAAAAANANASTSASNTVSGTVPVVPEPEVTSIVATVVDNENTVTISTEEQAQLTSTPAIQDQSVEVSSNTGEETSKQETVADFTPKKEEEESQPAKKTYTWNTKEEAKQAFKELLKEKRVPSNASWEQAMKMIINDPRYSALAKLSEKKQAFNAYKVQTEKEEKEEARSKYKEAKESFQRFLENHEKMTSTTRYKKAEQMFGEMEVWNAISERDRLEIYEDVLFFLSKKEKEQAKQLRKRNWEALKNILDNMANVTYSTTWSEAQQYLMDNPTFAEDEELQNMDKEDALICFEEHIRALEKEEEEEKQKSLLRERRRQRKNRESFQIFLDELHEHGQLHSMSSWMELYPTISSDIRFTNMLGQPGSTALDLFKFYVEDLKARYHDEKKIIKDILKDKGFVVEVNTTFEDFVAIISSTKRSTTLDAGNIKLAFNSLLEKAEAREREREKEEARKMKRKESAFKSMLKQAAPPIELDAVWEDIRERFVKEPAFEDITLESERKRIFKDFMHVLEHECQHHHSKNKKHSKKSKKHHRKRSRSRSGSDSDDDDSHSKKKRQRSESRSASEHSSSAESERSYKKSKKHKKKSKKRRHKSDSPESDAEREKDKKEKDRESEKDRTRQRSESKHKSPKKKTGKDSGNWDTSGSELSEGELEKRRRTLLEQLDDDQ

SEQ ID NO: 59 - DNA, 7528 bp, Homo sapiens
  ref <308>: DATABASE ACCESSION NUMBER: NCBI / NM_017892.3
  ref <309>: DATABASE ENTRY DATE: 2011-09-17
  ref <313>: RELEVANT RESIDUES IN SEQ ID NO: (1)..(7528)
actcagatcgcttcttgttggtatgtgtagcggcagtggccgccggcggagcagtctgagcccgacgatgaggccggggacgggagctgagcgtggaggcctcatgatggggcaccctggcatgcattatgccccaatgggaatgcaccctatgggtcagagagcgaatatgcctcctgtacctcatggaatgatgccgcagatgatgccccctatgggagggccaccaatgggacaaatgcctggaatgatgtcgtcagtaatgcctggaatgatgatgtctcatatgtctcaggcttccatgcagcctgccttaccgccaggagtaaatagtatggatgtagcagcaggtacagcatctggtgcaaaatcaatgtggactgaacataaatcacctgatggaaggacttactactacaacactgaaaccaaacagtctacctgggagaaaccagatgatcttaaaacacctgctgagcaactcttatctaaatgcccctggaaggaatacaaatcagattctggaaagccttactattataattctcaaacaaaagaatctcgctgggccaaacctaaagaacttgaggatcttgaaggataccagaataccattgttgctggaagtcttattacaaaatcaaacctgcatgcaatgatcaaagctgaagaaagcagtaagcaagaagagtgcaccacaacatcaacagccccagtccctacaacagaaattccgaccacaatgagcaccatggctgctgccgaagcagcagctgctgttgttgcagcagcagcagcggcagcagcagcagcagctgcagccaatgctaatgcttccacttctgcttctaatactgtcagtggaactgttccagttgttcctgagcctgaagttacttccattgttgctactgttgtagataatgagaatacagtaactatttcaactgaggaacaagcacaacttactagtacccctgctattcaggatcaaagtgtggaagtatccagtaatactggagaagaaacatctaagcaagaaactgtagctgattttactcccaaaaaagaagaggaggagagccaaccagcaaagaaaacatacacttggaatacaaaggaagaggcaaagcaagcttttaaagaattattgaaagaaaagcgggtaccatcgaatgcttcatgggagcaggctatgaaaatgattattaatgatccacgatacagtgctttggcaaagttaagtgaaaaaaagcaagcctttaatgcctataaagtccagacagaaaaagaagaaaaagaagaagcaagatcaaagtacaaagaggctaaggaatcctttcagcgttttcttgaaaatcatgagaaaatgacttctacaaccagatacaaaaaagcagagcaaatgtttggagagatggaagtttggaatgcaatatcagaacgtgatcgtcttgaaatctatgaagatgttttgttctttctttcaaaaaaagaaaaggaacaagcaaagcagttgcgaaagagaaattgggaagccttaaaaaacatacttgacaacatggctaatgtaacatactctaccacttggtctgaagcccagcagtatctgatggataatccaacttttgcagaagatgaggagttacaaaatatggacaaagaagatgcattaatttgctttgaagaacacattcgggctttagaaaaggaggaagaagaagaaaaacagaagagtttgctgagagaaaggagacgacagcgaaaaaatagggaatctttccagatatttttagatgaattacatgaacatggacaactgcattctatgtcatcttggatggaattgtatccaactattagttctgatattagattcactaatatgcttggtcagcctggatcaactgcacttgatcttttcaagttttatgttgaggatcttaaagcacgttatcatgacgagaagaagataataaaagacattctaaaggataaaggatttgtagttgaagtaaacactacttttgaagattttgtggcgataatcagttcaactaaaagatcaactacattagatgctggaaatatcaaattggctttcaatagtttactagaaaaggcagaagcccgtgaacgtgaaagagaaaaagaagaggctcggaagatgaaacgaaaagaatctgcatttaagagtatgttaaaacaagctgctcctccgatagaattggatgctgtctgggaagatatccgtgagagatttgtaaaagagccagcatttgaggacataactctagaatctgaaagaaaacgaatatttaaagattttatgcatgtgcttgagcatgaatgtcagcatcatcattcaaagaacaagaaacattctaagaaatctaaaaaacatcataggaaacgttcccgctctcgatcggggtcagattcagatgatgatgatagccattcaaagaaaaaaagacagcgatcagagtctcgttctgcttcagaacattcttctagtgcagagtctgagagaagttataaaaagtcaaaaaagcataagaagaaaagtaagaagaggagacataaatctgactctccagaatccgatgctgagcgagagaaggataaaaaagaaaaagatcgggaaagtgaaaaagacagaactagacaaagatcagaatcaaaacacaaatcgcctaagaaaaagactggaaaggattctggtaattgggatacttctggcagcgaactgagtgaaggggaattggaaaagcgcagaagaacccttttggagcaactggatgatgatcaataaattataccaaatatatgtttacagtatgatttaaagtctgattcagaccagggactctattttaagttcaactgaaataacactgggttttaattatatcacaggaaaaaaaaagtgcatttaagtattgttatcgtggactttataaaagcaaaggaaattgaaagtaacttttgattctgtatcaagaatcatattttcatacagtcataactgtctttctgtgaccctttcacagggcactgtaggatggattaaaggtggcaatttactgataactgcagatgtctctactttgttctaaaatctaagtcatgaggtgatttgatttactttatagaagctggattttgaagatctaatgaaaaattttttgataatatagtagtacaaaaaaagcaccagcaactgataaaaattgcttttttgtgcgctacccaactggttaaagccaatgtgatcttttatggtgaaactcctaagaaacaggtggttttgctggaaacttggtagacccttaattatagtggtgctaatgagcactactgtaatataaagccaccattattttttatcaaacatctgaatacattttacaaaggctattgtgagggcattattttgagcatctattttgaggtgatgtttaaaaaaactttaacatcaaatcaaattgtaaattaatttaaatatattgccttaaggacctactaaagaatgtgccaccagactttaagtgatagttgcaatatccttgtctaaaaaaaaaaaaaaagttgacttaaacattttctttaacagttgtcttttttttctaaattcagtctttctcttgcttttttttccctgctattgaggaagtattttgccttccctactcactgagaagtattgacttcgtggtacacattctaaagcatttctgatttgaatatttttgtacatttttatcaattattaaaccttctcttctagtgtgtactagtatttatttctacttaaactgctcagctctaaagaaattgtatgatcactttaaaggtactaaatccctgaaattttgcttcatcttacccacaccgctagtttactttccttttttaatatttaagaatattataaatcagtgcatttgaggtccctagatttgtttttcacctatgaattttttacagactattttagagaggctgtggtttgaagacagaatactctttacatgctaacttttgaaggttggtaatgtaatttatactattttccttcagtgcaggagattttttaagtaaataagtaaattttagcacctattcttcggtattatctgtgttattcttttaatgtgtacacttgaagagatcctatttcaaagttgggattttgaagaacaaaaagtttttttcttacccatcctagtgagttttgaaagtgggcctgaccagaatgtctctttcccattttgccccgtttgaattaaatataatgtctactctttaaaggctgaaggggtgggtgaggggattgtttctcattttgtctcccaagtcatttttctgctgtgaaatatgaccaggcttgtaggaagactcatcttggagaaaatgtgaagtaatcaaattgcttgagattagtctcctattgtatattagagccaaaacacataataactttggcaacagggagttgtctagactcaattttcagaggtcccattgtagtgagttaatattgctaatcatgtcaatcacttgacatggaagtcagtggcaaatctttaaagtatgcatttgatactggcaaatatatactactgatgtttcacaaaagaatcttagaatctgtagaaaacattaattacttccatgaattatttctaaagtataactttaaagttttgatttttctatttaaataaaaagctattgatgtgttttagcatgtcctcaaaatagattcctgtatttcttacagagataatcttactataaccgcaaatccctacaacacagtataaagtttccctgtagttcacccatgaaaattatttgacttaaaaacaaccatagtagtaagccaattgcaattcaaaatgcaacatccttgccataaaagatgtaacttaagtgactgtgatcctttgtctacctttcaggatcccatcaaatgggatttagttgctagaaagatacttgacagtctgaaggcacaggtgataagattttgttctctgtgaatatttttaaactaataggttatgtttttaaatcttcaatttggtaaaaaactgaaacgtcttggaatattggttctattagagttaaatatggtaaaacaggaaagcgtgtttattttcagataaggggaatttatacttttcattaaagtcttggagtcttaggaaaatgttgagggtttgtcttcccccagtgtcattaatactaatgaactattagatcaaattcacttatacatttaatatcatagttattcaaaatcaggactttaaatatttttttctcccagttcatatttctttgtgcgaaaattgaatttgtatgaaaagttagataattaagctaatcaagtatgaagtatacactcttcctaagtgaatttaaataatgtaatgcttttaaataaaagcagtggtcccaccacatttttttcagcatagtagctggcattttgttatcattagccagagtagcttcagcatataagttattgatgttttccaacttcaaaagtgcattttatgcctcccagtaaatttgataatgatttgtcacagctttgtcatcttttgacttttgcttatgggcctcacttcgtacaactataacatgaaaaaggattgtcctaaagtaagggaatcagttaatggtaggatgaagaaactgtaaaaactcctagaaaaaaacctgtgtgcatttttctggaaagttttcaaactgtgtaattcagttttcattcaattatataatttggttatatgctttaaaaaacatttgtctaaatgttcccggttttcttctggtcttagagtcagctgagtgctggctatgcagccactcgtatttttgcatccagaaaggagtaactccctttatatgaagatttttttttttaagcttagatgctatgtaagagaaaactatttgtaatcacatagtacccagggaggagtgatgaatgcttttttaaaaaaggatatttaagtatattatgtaatttaaatataaattatcaaactaaaaaacttcagcttttaaaactttctaaacaaaatgaagcaaagtacattaattccttctatcttaaatctgagactttttattttcccttgggtatatatttctctcagtgttaggtagatgttaattcaaccatcctacattctctatataaaattacactcattttacatgaaagcaaaataatgtcgttactttcatggaactgtgttctaacaggcttataatctcattctgtgcttctggtaatggcgtataccagtctgatgattcttaccatccttattcttacattactcttccaattgttttgattttaactggtggtcatgtaagttcctcggtggcagggatttatctggttcatttactgttatatatccttgtggaataggacagtgcctggcatataataggcacttttggaatgaatgtaaaatactagttgaaatatgtattcatctggcatccatggtacccatcatctgtattaaagggcttaaaaaagcatcattgcaataaaccatactaacccaccactaaagtcacagtcataattgaagtgcacccaaaataaggtccagtgactgtaccttagcttttacttcagacatgtccaaaacttgtgagaacatacaatttaaagttgaaaactcatcattttgggctatttttgaatagggctgaaatatggtgacatgtcaggcactcagttttttggagctgtcttaaaggtgaggtggaaagtgagagatgattttgctctaattacccacacattctccctaaccctccattttaagacacttagctataatagcttaagactatcaaaactgcttttgtgattctcagtaggatctagttcaaagccatctcagtgctagtgattggtaattgttaatttgatggtaattaccctacttgtaaagttgacattcatactggctaatcctccctgcccctagtcgtggacaatgaaacagtgagtggctggtgaattagatcagattcaaactaagccattccttgatgctattcttcactgctgttccttagggtatctaaatgtgttatttataaacatttttgggggctcctgcctgcaaacttcggtaggtgttggtcttccccacctctacaactgagccaaggacttgttctgaaaggtgagtaataaataagggaacaaggtttctattttctgtgtttgtaattaccagattttcacctctgtttatatattattctgatgtaaaagaacctcatagttatataaaattagaccttttgtaaataaatataatttattagaaaaaatggtatcttttactaaattggatttgtgtcagttttttcctttaagcaccactcaggcagcaagctctatgctgtgagagttgctggaggcccacgttatgttttctcagtgtaattcctctttttcttgataaaaaga